(19) United States
(12) Patent Application Publication
(10) Pub. No.: US 2006/0252782 A1
(43) Pub. Date: Nov. 9, 2006

(54) Sulfoximine-macrocycle compounds and salts thereof, pharmaceutical compositions comprising said compounds, methods of preparing same and uses of same

(76) Inventors: Ulrich Luecking, Berlin (DE); Georg Kettschau, Berlin (DE); Hans Briem, Bremen (DE); Wolfgang Schwede, Glienicke (DE); Martina Schaefer, Berlin (DE); Karl-Heinz Thierauch, Berlin (DE); Manfred Husemann, Hohen Neuendorf (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/399,619

(22) Filed: Apr. 7, 2006

Related U.S. Application Data
(60) Provisional application No. 60/670,640, filed on Apr. 13, 2005.

(30) Foreign Application Priority Data
Apr. 8, 2005 (EP) 05090098.4

(51) Int. Cl. A61K 31/519; C07D 487/14

(52) U.S. Cl. 514/267; 540/456

(57) ABSTRACT

The invention relates to macrocyctic sulfoximines of the general Formula I: 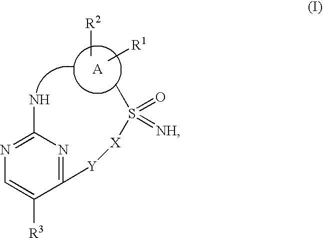
wherein A, X, Y, R¹, R² and R³ have the meaning as given in the specification and the claims, and to salts thereof; to pharmaceutical compositions comprising the macrocyclic sulfoximines, and to a method of preparing the macrocyclic sulfoximines, as well as the use thereof for manufacturing a pharmaceutical composition for the treatment of diseases of dysregulated vascular growth or of diseases which are accompanied with dysregulated vascular growth, wherein the compounds effectively interfere with angiopoietin and therefore influence Tie2 signalling.

This application claims the benefit of the filing date of U.S. Provisional Application Ser. No. 60/670,640 filed Apr. 13, 2005 which is incorporated by reference herein.

The invention relates to macrocyclic sulfoximines and salts thereof, to pharmaceutical compositions comprising said macrocyclic sulfoximines and to methods of preparing said macrocyclic sulfoximines, as well as to uses of said macrocyclic sulfoximes.

In order to defeat diseases with dysregulated vascular growth such as cancer different strategies were developed. One possible strategy is the blockade of angiogenesis to the tumour tissue, because tumour angiogenesis is a prerequisite for the growth of solid tumours.

The angiogenesis represents beside the vasculogenesis one of two basic processes during the genesis of vasculature. Vasculogenesis names the neoplasm of vasculature during the embryo development, wherein the angiogenesis describes the neoplasm of vasculature by sprouts or division of present vasculature. It has been found that two receptors expressed on endothelial cells, VEGF- (vascular endothelial growth factor) and Tie-receptors (also called Tek), are essential for normal development of vascular tissue as blood vessels (Dumont et al., “Dominant-negative and targeted null mutations in the endothelial receptor tyrosine kinase Tie-2 reveal a critical role in vasculogenesis of the embryo.”, Genes Dev, 1994, 8:1897-909; Sato et al.: “Distinct roles of the receptor tyrosine kinases Tie-1 and Tie-2 in blood vessel formation.”, Nature, Jul 6, 1995; 376(6535):70-4.).

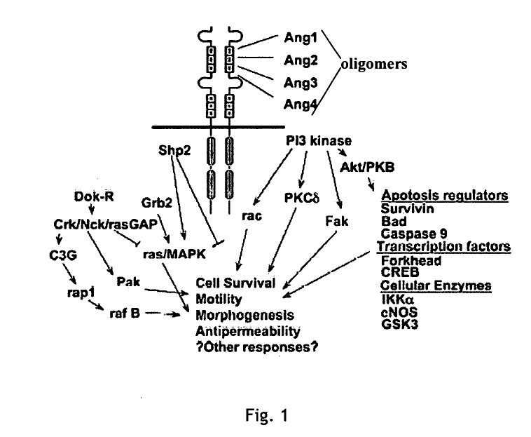

The mechanism of Tie2 signalling was characterized by different researchers, wherein different angiopoietins were found to be involved. So it could be explained that angiopoietin-1 if bound to the extracellular domain of the Tie2-receptor stimulates autophosphorylation and activates the intracellular kinase domain. Angiopoietin-1 activation of Tie2 however does not stimulate mitogenesis but rather migration. Angiopoietin-2 can block angiopoietin-1 mediated Tie2 activation and the resulting endothelial migration. This indicates that angiopoietin-2 is a naturally occurring inhibitor of Tie2 activation (Maisonpierre et al.: “Angiopoietin-2, a natural antagonist for Tie2 that disrupts in vivo angiogenesis.”, Science, Jul. 4, 1997; 277(5322):55-60; Witzenbichler et al.: “Chemotactic properties of angiopoietin-1 and -2, ligands for the endothelial-specific receptor tyrosine kinase Tie2.”, J. Biol Chem., Jul. 17, 1998; 273(29):18514-21). For an overview see FIG. 1 modified by Peters et al. (Peters et al.: “Functional significance of Tie2 signalling in the adult vasculature”. Recent Prog Horm Res. 2004; 59:51-71. Review.).

Receptor dimerization results in cross-phosphorylation on specific tyrosine-residues. Receptor cross-phosphorylation has a dual effect: it enhances the receptor's kinase activity and it provides binding sites for signalling molecules possessing phosphotyrosine binding domains (SH2 and PTB domains) (Pawson T.: “Regulation and targets of receptor tyrosine kinases”, Eur J Cancer. September 2002, 38 Suppl 5:S3-10. Review).

The signalling cross-talk between the P13-K pathway and the Dok-R pathway is required for an optimal chemotactic response downstream of Tie2. Other recent studies have shown that Tie2-mediated activation of the P13-K/Akt pathway is required for endothelial nitric oxide synthase (eNOS) activation, focal adhesion kinase activation, and protease secretion, all of which may contribute importantly to Tie2 function during angiogenesis (Kim I. et al.: “Angiopoietin-1 regulates endothelial cell survival through the phosphatidylinositol 3′-Kinase/Akt signal transduction pathway”, Circ Res. Jan. 7-21, 2000; 86(1):24-9; Babaei et al.: “Angiogenic actions of angiopoietin-1 require endothelium-derived nitric oxide”, Am J Pathol. June 2003; 162(6):1927-36).

For normal development a balanced interaction between the receptors and so-called ligands is necessary. Especially the angiopoietins, which signal via Tie2 receptors, play an important role in angiogenesis (Babaei et al., 2003).

The broad expression of Tie2 in adult vasculature has been confirmed in transgenic mice using Tie2 promoter driven reporters (Schlaeger et at.: “Uniform vascular-endothelial-cell-specific gene expression in both embryonic and adult transgenic mice”, Proc Natl Acad Sci USA. Apr. 1, 1997; 94(7):3058-63; Motoike et al.: “Universal GFP reporter for the study of vascular development”, Genesis, October 2000; 28(2):75-81). Immunohistochemical analysis demonstrated the expression of Tie2 in adult rat tissues undergoing angiogenesis. During ovarian folliculogenesis, Tie2 was expressed in the neo-vessels of the developing corpus luteum. Angiopoietin-1 and angiopoietin-2 also were expressed in the corpus luteum, with angiopoietin-2 localizing to the leading edge of proliferating vessels and angiopoietin-1 localizing diffusely behind the leading edge (Maisonpierre et al., 1997). It was suggested that angiopoietin-2-mediated inhibition of Tie2 activation serves to “destabilize” the vessel, to make it responsive to other angiogenic growth factors such as VEGF. Subsequently, angiopoietin-1-mediated activation of Tie2 would trigger stabilization of the neovasculature.

The disruption of Tie2 function shows the relevance of Tie2 for neoangiogenesis in transgenic mice resulting in early embryonic lethality as a consequence of vascular abnormalities (Dumont et al., 1994; Sato et al., 1995). Tie2−/− embryos failed to develop the normal vessel hierarchy, suggestive of a failure of vascular branching and differentiation. Tie2−/− embryos have a decreased number of endothelial cells and furthermore less contact between endothelial cells and the underlying pericytes/smooth muscle cells. This implies a role in the maturation and stabilization of newly formed vasculature.

The studies in mice with transgenic or ablated Tie2 gene suggest a critical role for Tie2 in maturation of vascular development in embryos and in adult vasculature. Conditional expression of Tie2 in the endothelium of mice homozygous for a Tie2 null allele partially rescued the embryonic lethality of the Tie2 null phenotype (Jones N et al.: “Tie receptors: new modulators of angiogenic and lymphangiogenic responses”, Nat Rev Mol Cell Biol., April 2001; 2(4):257-67. Review). Mice lacking functional angiopoietin-1 expression and mice over-expressing angiopoietin-2 both displayed a phenotype similar to Tie2−/− mice (Suri et al.: “Requisite role of angiopoietin-1, a ligand for the Tie2 receptor, during embryonic angiogenesis”, Cell, Dec. 27, 1996; 87(7): 1171-80; Maisonpierre PC et al.: “Angiopoietin-2, a natural antagonist for Tie2 that disrupts in vivo angiogenesis”, Science, Jul. 4, 1997; 277(5322):55-60.).

Angiopoietin-2−/− mice have profound defects in the growth and patterning of lymphatic vasculature and fail to remodel and regress the hyaloid vasculature of the neonatal lens (Gale et al.: “Angiopoietin 2 is required for postnatal angiogenesis and lymphatic patterning, and only the latter role is rescued by Angiopoietin-1”, Dev Cell. September 2002; 3(3):411-23). Angiopoietin-1 rescued the lymphatic defects, but not the vascular remodeling defects. So angiopoietin-2 might function as a Tie2 antagonist in blood vasculature but as a Tie2 agonist in developing lymph vasculature.

Tie2 also plays a role in pathological angiogenesis. It was shown that mutations in Tie2 cause inherited venous malformations and enhance both ligand dependent and independent Tie2 kinase activity (Vikkula et al.: “Dysmorphogenesis caused by an activating mutation in the receptor tyrosine kinase Tie2”, Cell, Dec. 27, 1996; 87(7):1181-90). Tie2 expression was investigated in human breast cancer tumour specimens and Tie2 expression was found in the vascular endothelium both in normal breast tissue and in breast tumours. The proportion of Tie2-positive tumour microvessels was increased in tumours as compared to normal breast tissue (Peters KG et al.: “Expression of Tie2/Tek in breast tumour vasculature provides a new marker for evaluation of tumour angiogenesis”, Br J Cancer, 1998, 77(1):51-6).

Angiopoietin-1 overexpression in tumour models resulted in decreased tumour growth. The effect is possibly related to angiopoietin-1 mediated stabilization of the tumour vasculature, which renders the vessels resistant to angiogenic stimuli (Hayes et al.: “Expression and function of angiopoietin-1 in breast cancer”, Br J Cancer, November 2000; 83(9):1154-60; Shim et al.: “Inhibition of angiopoietin-1 expression in tumour cells by an antisense RNA approach inhibited xenograft tumour growth in immunodeficient mice”, Int J Cancer, Oct. 1, 2001; 94(1):6-15; Shim et al.: “Angiopoietin 1 promotes tumour angiogenesis and tumour vessel plasticity of human cervical cancer in mice”, Exp Cell Res., Oct. 1, 2002; 279(2):299-309; Hawighorst et al.: “Activation of the tie2 receptor by angiopoietin-1 enhances tumour vessel maturation and impairs squamous cell carcinoma growth”, Am J Pathol. April 2002, 160(4):1381-92; Stoeltzing et al.: “Angiopoietin-1 inhibits vascular permeability, angiogenesis, and growth of hepatic colon cancer tumours”, Cancer Res. Jun. 15, 2003; 63(12):3370-7.).

Corneal angiogenesis induced by tumour cell conditioned medium was inhibited by recombinant sTie, despite the presence of VEGF. Mammary tumour growth was significantly inhibited in a skin chamber tumour model recombinant sTie2 (Lin et al.: “Inhibition of tumour angiogenesis using a soluble receptor establishes a role for Tie2 in pathologic vascular growth”, J Clin Invest., Oct. 15, 1997; 100(8):2072-8; Lin et al.: “Antiangiogenic gene therapy targeting the endothelium-specific receptor tyrosine kinase Tie2”, Proc Natl Acad Sci USA, Jul. 21, 1998; 95(15):8829-34). Similar sTie constructs have shown comparable effects in different tumour models (Siemeister et al.: “Two independent mechanisms essential for tumour angiogenesis: inhibition of human melanoma xenograft growth by interfering with either the vascular endothelial growth factor receptor pathway or the Tie-2 pathway”, Cancer Res. Jul. 1, 1999; 59(13):3185-91; Stratmann et al.: “Differential inhibition of tumour angiogenesis by Tie2 and vascular endothelial growth factor receptor-2 dominant-negative receptor mutants”, Int J Cancer. Feb. 1, 2001; 91 (3):273-82; Tanaka et al.: “Tie2 vascular endothelial receptor expression and function in hepatocellular carcinoma”, Hepatology. April 2002; 35(4):861-7).

When the interaction of angiopoietin-2 with its receptor is blocked by application of a neutralizing anti-angiopoietin-2 monoclonal antibody, the growth of experimental tumours can be blocked efficiently again pointing to the important role of Tie2 in tumour angiogenesis and growth (Oliner et al.: “Suppression of angiogenesis and tumour growth by selective inhibition of angiopoietin-2”, Cancer Cell. November 2004; 6(5):507-16.) So inhibiting the Tie2 pathway will inhibit pathological angiogenesis.

To influence the interaction between receptor and ligand it could be shown that angiogenesis may be blocked with blockers such as Avastin which interfere with VEGF signal transduction to endothelial cells.

Avastin is a clinically effective antibody that functions as tumour growth inhibitor by blockade of VEGFR mediated angiogenic signalling. Thus interference with VEGF signalling is a proven clinical principle. VEGF-C is a molecule inducing lymph angiogenesis via VEGFR 3. The blockade of this signal pathway is inhibiting diseases associated with lymph angiogenesis as is lymphoedema and related diseases (Saharinen et al.: “Lymphatic vasculature: development, molecular regulation and role in tumour metastasis and inflammation”, Trends Immunol., July 2004: 25(7): 387-95. Review).

Pyrimidines and their derivatives have been frequently described as therapeutic agents for diverse diseases. A series of recently published patent applications describes their use as inhibitors of various protein kinases, for example WO 2003/032997 A, WO 2003/063794 A, WO 2003/076437 A and WO 2002/096888 A. More specifically, certain pyrimidine derivatives have been disclosed as inhibitors of protein kinases involved in angiogenesis, such as VEGF or Tie2, for example benzimidazole substituted 2,4-diaminopyrimidines (WO 2003/074515 A) or (bis)anilino-pyrimidines (WO 2003/066601 A). Very recently, pyrimidine derivatives in which the pyrimidine constitutes a part of a macrocyclic ring system, have been reported to be inhibitors of CDKs and/or VEGF (WO 2004/026881 A), or of CDK2 and/or CDK5, respectively (WO 2004/078682 A).

A particular problem in using such known substances as inhibitors or blockers is that their use at the same time is often accompanied with undesired cytotoxic side effects on normal developing and proliferating tissue. This originates from substances which are less selective and at the same time dose tolerability problems.

Therefore the aim of the present invention is to provide compounds, which are useful for the treatment of diseases of dysregulated vascular growth or diseases which are accompanied by dysregulated vascular growth. Furthermore, the prior art problems shall be prevented, especially compounds shalt be provided, which show low toxic side effects on normal proliferating tissue but are effectively inhibiting endothelial cell migration at small concentrations. This will further reduce undesired side effects.

The solution to the above-mentioned novel technical problems is achieved by providing compounds derived from a class of macrocyclic sulfoximines (hereinafter referred to as “sulfoximine-macrocycles”) and solvates, hydrates, N-oxides, isomers and salts thereof, methods of preparing sulfoximine-macrocycles, pharmaceutical compositions comprising said compounds, uses of said compounds and a method for treating diseases with said compounds, all in accordance with the description, as defined in the claims of the present Application.

The invention relates to compounds of the general Formula I:

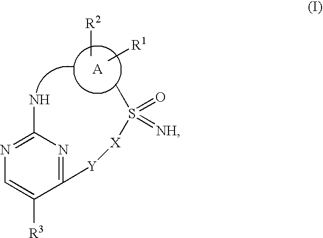

in which
A is -phenylene- or —C₅-C₆-heteroarylene-;
x is a 2- to 6-membered alkylene tether which is unsubstituted or singly or multiply substituted with one or more substituents selected from the group comprising, preferably consisting of, halogen, C₁-C₄-alkoxy, hydroxy, amino, cyano, carboxy, -NH-C₁-C₄-alkyl, —N(C₁-C₄-alkyl)₂, and C₁-C₄-alkyl, which itself may be unsubstituted or singly or multiply substituted by halogen, C₁-C₄-alkoxy, hydroxy, amino, cyano, carboxy, —NH—C₁-C₄-alkyl, or —N(C₁-C₄-alkyl)₂;
Y is —NR⁴—, —O—, or —S—;
R¹ and R² are the same or different and are independently from each other selected from the group comprising, preferably consisting of, hydrogen, hydroxy, halogen, nitro, cyano, —(CH₂)ₚP(O)(OR⁵)₂, —(CH₂)ₚOP(O)(OR⁵)₂—(CH₂)ₚ—NR⁵R⁶, —(CH₂)ₚ—NR⁵C(O)R⁶, —(CH₂)ₚ—NR⁵C(S)R⁶, —(CH₂)ₚ—NR⁵S(O)R⁶, —(CH₂)ₚ—NR⁵S(O)₂R⁶, —(CH₂)ₚ—NR⁵C(O)NR⁶R⁷, —(CH₂)ₚ—NR⁵C(O)OR⁶, —-(CH₂)ₚ—NR⁵C(NH)NR⁶R⁷, —(CH₂)ₚ—NR⁵C(S)NR⁶R⁷, —(CH₂)ₚ—NR⁵S(O)NR⁶R⁷, —(CH₂)ₚ—NR⁵S(O)₂NR⁶R⁷, —(CH₂)ₚ—C(O)R⁵, ——(CH₂)ₚ—CR⁴(OH)—R⁵, —(CH₂)ₚ—C(S)R⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—S(O)₂N═CH—NR⁵R⁶, —(CH₂)ₚ—SO₃R⁵, —(CH₂)ₚ—CO₂R⁵, —(CH₂)ₚ—C(O)NR⁵R⁶, —(CH₂)ₚ—C(S)NR⁵R⁶, —(CH₂)ₚ—SR⁵, —(CH₂)ₚ—OR⁵, —CR⁵(OH)—R⁶, —O—(CH₂)ₚ—C(O)R⁵, —O—(CH₂)ₚ—CR⁴(OH)—R⁵, and moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)p-phenyl, —(CH₂)ₚ—C₅-C₆-heteroaryl, -phenylene-(CH₂)ₚ—R⁵, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl, and wherein 0 to 2 methylene groups of —C₁-C₈-alkyl may be replaced independently of each other by —O—, —S—, —C(═O)—, —SO₂— or —NR⁴—;
R³ is selected from the group comprising, preferably consisting of, halogen, nitro, cyano, —C₁-C₆-alkylthio, amino, —NH—(CH₂)ₚ—C₃-C₈-cycloalkyl, benzodioxolyl, —(CH₂)ₚP(O)(OR⁵)₂, —NR⁵R⁶, —S(O)(C₁-C₆-alkyl), —S(O)₂(C₁-C₆-alkyl), —C(O)NR⁵R⁶, —C(O)R⁵, —C(O)OR⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₁₀-heteroaryl, —O-phenyl, —O—C₅-C₁₀-heteroaryl, -phenylene-(CH₂)ₚ—R⁵, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —NH—(CH₂)ₚ—C₃-C₈-cycloalkyl, —C₃-C₈-cycloalkyl, —C₃-C₈-heterocycloalkyl, —(CH₂)ₚ-phenyl, —(CH₂)ₚ—C₅-C₆-heteroaryl, -phenylene-(CH₂)ₚ—R⁵, —NR⁵R⁶, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —C₁-C₆-alkoxy-C₁-C₆-alkoxy-C₁-C₆-alkyl, —(CH₂)ₚP(O)(OR⁵)₂, —(CH₂)ₚOP(O)(OR⁵)₂, —(CH₂)ₚ—NR⁵C(O)NR⁶R⁷, —(CH₂)ₚ—S(O)R⁵, —(CH₂)ₚ—S(O)₂R⁵, —(CH₂)ₚ—NR⁵S(O)₂R⁶, —(CH₂)ₚ—S(O)₂NR⁵R⁶, —(CH₂)ₚ—C(O)NR⁵R⁶, —(CH₂)ₚ—NR⁵C(O)R⁶, —(CH₂)ₚ—CR⁴(OH)—R⁵, —O—(CH₂)ₚ—CR⁴(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein phenylene, —C₃-C₈-cycloalkyl, —C₃-C₈-heterocycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —SO₂— or —NR⁴—;
R⁴ is hydrogen or —C₁-C₈-alkyl;
R⁵, R⁶,
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₂-C₈-alkenyl, —C₂-C₈-alkynyl, -C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —NH—(CH₂)ₚ—C₃-C₈-cycloalkyl, —C₃-C₈-cycloalkyl, —C₃-C₈-heterocycloalkyl, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —C₁-C₆-alkoxy-C₁-C₆-alkoxy-C₁-C₆-alkyl, —NR⁹R¹⁰, —NR⁹C(O)NR¹⁰, —S(O)(C₁-C₆-alkyl), —S(O)₂(C₁-C₆-alkyl), —S(O)₂-phenyl, —NH—S(O)₂(C₁-C₆-alkyl), —NH—S(O)₂-phenyl, —S(O)₂—NH-(C₁-C₆-alkyl), —S(O)₂—NH-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₁₀-heteroaryl, -phenylene-(CH₂)ₚ—R⁹, (CH₂)ₚP(O)(OR⁹)₂, —(CH₂)ₚOP(O)(OR⁹)₂, wherein phenylene, —C₃-C₈-cycloalkyl, —C₃-C₈-heterocycloalkyl, phenyl, —(CH₂)ₚ-phenyl and —(CH₂)ₚ—C₅-C₁₀-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 2- to 8-membered alkylene tether which may be unsubstituted or singly substituted by —(CH₂)ₚ—OH, —(CH₂)ₚ—CN, or —(CH₂)ₚ—NR⁹R¹⁰, and in which 0 to 2 methylene groups may be replaced by —O—, —S—, —(C═O)—, —S(O)₂—, or —NR⁴—;
R⁹, R¹⁰ are independently from each other hydrogen or —C₁-C₈-alkyl;
p is an integer of 0, 1, 2, 3, 4, 5, or 6; and solvates, hydrates, N-oxides, isomers and salts thereof.

The following terms as mentioned herein and in the claims have preferably the following meanings:

The term “alkyl” is to be understood as preferably meaning branched and unbranched alkyl, meaning e.g. methyl, ethyl, n-propyl, iso-propyl, n-butyl, iso-butyl, tert-butyl, sec-butyl, pentyl, iso-pentyl, hexyl, heptyl, octyl, nonyl and decyl and the isomers thereof.

The term “haloalkyl” is to be understood as preferably meaning branched and unbranched alkyl, as defined supra, in which one or more of the hydrogen substituents is replaced in the same way or differently with halogen. Particularly preferably, said haloalkyl is, e.g. chloromethyl, fluoropropyl, fluoromethyl, difluoromethyl, trichloromethyl, 2,2,2-trifluoroethyl, pentafluoroethyl, bromobutyl, trifluoromethyl, iodoethyl, and isomers thereof.

The term “hydroxyalkyl” is to be understood as preferably meaning branched and unbranched alkyl, in which one or more of the hydrogen substituents is replaced in with a hydroxy group, e.g. hydroxymethyl, hydroxyethyl, 2-hydroxypropyl, 3-hydroxypropyl, 4-hydroxybutyl, 5-hydroxyhexyl, 3,4-dihydroxybutyl, 2-hydroxybutyl, 3-hydroxybutyl, 1-hydroxy-1-methylethyl and isomers thereof.

The term “alkoxy” is to be understood as preferably meaning branched and unbranched alkoxy, meaning e.g. methoxy, ethoxy, propyloxy, iso-propyloxy, butyloxy, iso-butyloxy, tert-butyloxy, sec-butyloxy, pentyloxy, iso-pentyloxy, hexyloxy, heptyloxy, octyloxy, nonyloxy, decyloxy, undecyloxy and dodecyloxy and the isomers thereof.

The term “haloalkoxy” is to be understood as preferably meaning branched and unbranched alkoxy, as defined supra, in which one or more of the hydrogen substituents is replaced in the same way or differently with halogen, e.g. chloromethoxy, fluoromethoxy, pentafluoroethoxy, fluoropropyloxy, difluoromethyloxy, trichloromethoxy, 2,2,2-trifluoroethoxy, bromobutyloxy, trifluoromethoxy, iodoethoxy, and isomers thereof.

The term “cycloalkyl” is to be understood as preferably meaning a C₃-C₁₀ cycloalkyl group, more particularly a saturated cycloalkyl group of the indicated ring size, meaning e.g. a cyclopropyl, cyclobutyl, cyclopentyl, cyclohexyl, cycloheptyl, cyclooctyl, cyclononyl, or cyclodecyl group; and also as meaning an unsaturated cycloalkyl group containing one or more double bonds in the C-backbone, e.g. a C₃-C₁₀ cycloalkenyl group, such as, for example, a cyclopropenyl, cyclobutenyl, cyclopentenyl, cyclohexenyl, cycloheptenyl, cyclooctenyl, cyclononenyl, or cyclodecenyl group, wherein the linkage of said cyclolakyl group to the rest of the molecule can be provided to the double or single bond.

The term “heterocycloalkyl” is to be understood as preferably meaning a C₃-C₁₀ cycloalkyl group, as defined supra, featuring the indicated number of ring atoms, wherein one or more ring atoms are heteroatoms such as nitrogen, oxygen or sulphur, or carbonyl groups, or, -otherwise stated—in a Cₙ-cycloalkyl group one or more carbon atoms are replaced by these heteroatoms to give such Cₙ cycloheteroalkyl group. Thus such group refers e.g. to a three-membered heterocycloalkyl, expressed as —C₃-heterocycloalkyl such as oxyranyl. Other examples of heterocycloalkyls are oxetanyl (C₄), aziridinyl (C₃), azetidinyl (C₄), tetrahydrofuranyl (C₅), pyrrolidinyl (C₅), morpholinyl (C₆), dithianyl (C₆), thiomorpholinyl (C₆), piperazinyl (C₆), trithianyl (C₆) and chinuclidinyl (C₈).

The term “halogen” or “Hal” is to be understood as preferably meaning fluorine, chlorine, bromine, or iodine.

The term “alkenyl” is to be understood as preferably meaning branched and unbranched alkenyl, e.g. a vinyl, propen-1-yl, propen-2-yl, but-1-en-1-yl, but-1-en-2-yl, but-2-en-1-yl, but-2-en-2-yl, but-i -en-3-yl, 2-methyl-prop-2-en-1-yl, or 2-methyl-prop-1-en-1-yl group.

The term “alkynyl” is to be understood as preferably meaning branched and unbranched alkynyl, e.g. an ethynyl, prop-1-yn-1-yl, but-1-yn-1-yl, but-2-yn-1-yl, or but-3-yn-1-yl group.

As used herein, the term “aryl” is defined in each case as having 3-12 carbon atoms, preferably 6-12 carbon atoms, such as, for example, cyclopropenyl, cyclopentadienyl, phenyl, tropyl, cyclooctadienyl, indenyl, naphthyl, azulenyl, biphenyl, fluorenyl, anthracenyl etc, phenyl being preferred.

As used herein, the term “heteroaryl” is understood as meaning an aromatic ring system which comprises 3-16 ring atoms, preferably 5 or 6 or 9 or 10 atoms, and which contains at least one heteroatom which may be identical or different, said heteroatom being such as oxygen, nitrogen or sulfur, and can be monocyclic, bicyclic, or tricyclic, and in addition in each case can be benzocondensed. Preferably, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, e.g., benzofuranyl, benzothienyl, benzoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

The term “alkylene”, as used herein in the context of the compounds of general formula (I) which include a group X, is to be understood as meaning an optionally substituted alkyl chain or “tether”, having 1, 2, 3, 4, 5, or 6 carbon atoms, i.e. an optionally substituted —CH₂— (“methylene” or “single membered tether”), —CH₂—CH₂— (“ethylene”, “dimethylene”, or “two-membered tether”), —CH₂—CH₂—CH₂— (“propylene”, “trimethylene”, or “three-membered tether”), —CH₂—CH₂—CH₂—CH₂— (“butylene”, “tetramethylene”, or “four-membered tether”), —CH₂—CH₂—CH₂—CH₂—CH₂— (“pentylene”, “pentamethylene” or “five-membered ether”), or —CH₂—CH₂—CH₂—CH₂—CH₂—CH₂— (“hexylene”, “hexamethylene”, or six-membered tether”) group. Preferably, said alkylene tether is 2, 3, 4, or 5 carbon atoms, more preferably 4 or 5 carbon atoms. The term “arylene” is to be understood as preferably meaning a monocyclic or polycyclic arylene aromatic system featuring the indicated number of ring atoms, e.g. to phenylene, naphthylene and biphenylene. If the term phenylene is used it should be understood that the linking residues can be arranged to each other in ortho-, para- or meta-position.

The term “heteroarylene” refers to monocyclic or polycyclic arylenes featuring the indicated number of ring atoms wherein one or more ring atoms are heteroatoms such as nitrogen, oxygen or sulphur or—otherwise stated—in a Cₙ-arylene group one or more carbon atoms are replaced by these heteroatoms to give such Cₙ-heteroarylene group. The term heteroarylene may be exemplified by but is not limited to e.g. to five-membered heteroarylene, expressed as —C₅-heteroarylene, such as thiophenylene, furanylene, oxazolylene, thiazolylene, imidazolylene, pyrazolylene, triazolylene, thia-4H-pyrazolylene; or six-membered heteroarylene, expressed as —C₆-heteroarylene, such as pyridinylene, pyrimidinylene, triazinylene, and benzo-derivates thereof such as quinolinylene and isoquinolinylene.

The term heteroarylene may be furthermore exemplified but is not limited to benzo-derivatives of said —C₅— and —C₆-heteroarylenes, such as indolylene, benzofuranylene, or benzimidazolylene, expressed as —C₉-heteroarylene; or quinolinylene and isoquinolinylene, expressed as —C₁₀-heteroaryl.

As used herein, the term “C₁-C₆”, as used throughout this text, e.g. in the context of the definition of “C₁-C₆-alkyl”, “C₁-C₆-alkoxy”, “C₁-C₆-alkylthio”, “C₁-C₆-hydroxyalkyl”, “C₁-C₆-haloalkyl”, “C₁-C₆-haloalkoxy”, or “C₁-C₆-alkanoyl”, etc., is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; preferably C₁-C₂, C₁-C₃, C₁-C₄, C₁-C₅, C₁-C₆; more preferably C₁-C₄. The term “C₁-C₈” is to be interpreted as having the respective definition as above, i.e. as a group having a finite number of carbon atoms of 1 to 8, i.e. 1, 2, 3, 4, 5, 6, 7, or 8 carbon atoms, and as comprising the respective sub-ranges contained therein.

As used herein, the term “C₁-C₄”, as used throughout this text, e.g. in the context of the definition of “C₁-C₄-alkyl”, “C₁-C₄-alkoxy”, “C₁-C₄-haloalkyl”, “C₁-C₄-haloalkoxy”, or “C₁-C₄-hydroxyalkyl”, etc., is to be understood as meaning an alkyl group having a finite number of carbon atoms of 1 to 4, i.e. 1, 2, 3, or 4 carbon atoms. It is to be understood further that said term “C₁-C₄” is to be interpreted as any preferable sub-range comprised therein, e.g. C₁-C₄, C₂-C₃, C₁-C₂, C₁-C₃, C₂-C₄.

As used herein, the term “C₃-C₈”, as used throughout this text, e.g. in the context of the definitions of “C₃-C₈-cycloalkyl” or “C₃-C₈-heterocycloalkyl”, is to be understood as meaning a cycloalkyl group having a finite number of carbon atoms of 3 to 8, i.e. 3, 4, 5, 6, 7, or 8 carbon atoms, preferably 3, 4, 5 or 6 carbon atoms. It is to be understood further that said term “C₃-C₈” is to be interpreted as any sub-range comprised therein, e.g. C₃-C₈, C₄-C₇, C₅-C₆; preferably C₃-C₆.

Similarly, as used herein, the term “C₂-C₈”, or “C₂-C₆” as used throughout this text e.g. in the context of the definitions of “C₂-C₈— or C₂-C₆-alkenyl or -alkynyl”, is to be understood as meaning an alkenyl or alkynyl group having a finite number of carbon atoms of 2 to 8, or 2 to 6, respectively, i.e. 2, 3, 4, 5, 6, 7 or 8 carbon atoms. It is to be understood further that said term “C₂-C₈” or C₂-C₆” is to be interpreted as any subrange comprised therein, e.g. C₂-C₈, C₂-C₇, C₂-C₆, C₃-C₅, C₃-C₄, C₂-C₃, C₂-C₄, C₂-C₅; preferably C₂-C₃.

As used herein, the term “C₅-C₁₀”, as used throughout this text, e.g. in the context of the definition of “C₅-C₁₀-heteroaryl”, is to be understood as meaning an aromatic ring system which contains, in the ring, at least one heteroatom, which may be identical or different, and which comprises 5 to 10 ring atoms, preferably 5 or 6 atoms, more preferably 9 or 10 ring atoms, said heteroatom being such as oxygen, nitrogen or sulphur, and can be monocyclic, bicyclic, or tricyclic, and cycloalkyl group having a finite number of carbon atoms of 5 to 10, i.e. 5, 6, 7, 8, 9 or 10 carbon atoms, preferably 5 or 6 carbon atoms. It is to be understood further that said term “C₅-C₁₀” is to be interpreted as any sub-range comprised therein, e.g. C₅-C₁₀, C₆-C₉ , C₇-C₈ preferably C₅-C₆.

The compound according to Formula I (sulfoximine-macrocycles) can exist as N-oxides which are defined in that at least one nitrogen of the compounds of the general Formula I may be oxidized.

The compound according to Formula I (sulfoximine-macrocycles) can exist as a solvate, in particular as a hydrate, wherein the compound according to Formula I may conain polar solvents, in particular water, as structural element of the crystal lattice of the compounds. The amount of polar solvents, in particular water, may exist in a stoichiometric or unstoichiometric ratio. In case of stoichiometric solvates, e.g. hydrate, hemi-, (semi-), mono-, sesqui-, di-, tri-, tetra-, penta- etc, solvates or hydrates, respectively, are possible.

The term “isomers” is understood as meaning chemical compounds with the same number and types of atoms as another chemical species. There are two main classes of isomers, constitutional isomers and stereoisomers.

The term “constitutional isomers” refers to chemical compounds with the same number and types of atoms, but they are connected in differing sequences. There are functional isomers, structural isomers, tautomers or valence isomers.

In “stereoisomers”, the atoms are connected sequentially in the same way, such that condensed Formulae for two isomeric molecules are identical. The isomers differ, however, in the way the atoms are arranged in space. There are two major sub-classes of stereoisomers; conformational isomers, which interconvert through rotations around single bonds, and configurational isomers, which are not readily interconvertable.

“Configurational isomers” are, in turn, comprised of enantiomers and diastereomers. Enantiomers are stereoisomers which are related to each other as mirror images. Enantiomers can contain any number of stereogenic centers, as long as each center is the exact mirror image of the corresponding center in the other molecule. If one or more of these centers differs in configuration, the two molecules are no longer mirror images. Stereoisomers which are not enantiomers are called diastereomers. Diastereomers, which still have a different constitution, are another sub-class of diastereomers, the best known of which are simple cis-trans isomers.

In order to limit different types of isomers from each other reference is made to IUPAC Rules Section E (Pure Appl Chem 45, 11-30, 1976).

The compound according to Formula I (sulfoximine-macrocycles) can exist in free form or in a salt form, wherein the salt can be a suitable pharmaceutically acceptable salt. A suitable pharmaceutically acceptable salt of the sulfoximine-macrocycles of the present invention can be, for example, an acid-addition salt of a sulfoximine-macrocycle of the invention which is sufficiently basic, for example, an acid-addition salt with, for example, an inorganic or organic acid, for example hydrochloric, hydrobromic, sulphuric, phosphoric, acetic, pivalic, propionic, lactic, trifluoroacetic, citric, tartaric, fumaric, malonic, malic, succinic, maleic acid, or methanesulfonic, ethanesulfonic, camphorsulphonic, benzenesulfonic, para-toluenesulphonic or naphthalenesulfonic acid. In addition a suitable pharmaceutically acceptable salt of a sulfoximine-macrocycle of the invention which is sufficiently acidic is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, 1,6-hexadiamine, ethanolamine, glucosamine, sarcosine, serinol, tris-hydroxy-methyl-aminomethane, aminopropandiol, sovak-base, 1-amino-2, 3,4-butantriol.

As used herein, the term “in vivo hydrolysable ester” is understood as meaning an in vivo hydrolysable ester of a compound of formula (I) containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of formula (I) containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of further in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

Compounds of the Formula I of the present invention are preferred, wherein
A is -meta-phenylene- or —C₅-C₆-heteroarylene-;
X is a 2- to 6-membered alkylene tether which is unsubstituted or singly or multiply substituted with one or more substituents selected from the group comprising, preferably consisting of, C₁-C₄-alkoxy, hydroxy, amino, —NH—C₁-C₄-alkyl, —N(C₁-C₄-alkyl)₂, or C₁-C₄-alkyl, which itself may be unsubstituted or singly or multiply substituted by C₁-C₄-alkoxy, hydroxy, amino, —NH—C₁-C₄-alkyl, or —N(C₁-C₄-alkyl)₂;
Y is —NR⁴— or —O—;
R¹ and R² are the same or different and are independently from each other selected from the group comprising, preferably consisting of, hydrogen, hydroxy, halogen, nitro, cyano, —(CH₂)ₚ—NR⁵R⁶, —(CH₂)ₚ—NR⁵C(O)R⁶, —(CH₂)ₚ—NR⁵S(O)₂R⁶, —(CH₂)ₚ—NR⁵C(O)NR⁶R⁷, —(CH₂)ₚ—NR⁵C(O)OR⁶, —(CH₂)ₚ—C(O)R⁵, —(CH₂)ₚ—CR⁴(OH)—R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —(CH₂)ₚ—OR⁵, —CR⁵(OH)—R⁶, —O—(CH₂)ₚ—C(O)R⁵, —O—(CH₂)ₚ—CR⁴(OH)—R⁵, and moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl, and wherein 0 to 2 methylene groups of —C₁-C₈-alkyl may be replaced independently of each other by —O—, —S—, —C(═O)—, —S(O)₂— or —NR⁴—;
R³ is selected from the group comprising, preferably consisting of, halogen, nitro, cyano, —C₁-C₆-alkylthio, amino, benzodioxolyl, —NR⁵R⁶, —S(O)₂(C₁-C₆-alkyl), —C(O)NR⁵R⁶, —C(O)R⁵, —C(O)OR⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₁₀-heteroaryl, —O-phenyl, —O—C₅-C₁₀-heteroaryl, -phenylene-(CH₂)ₚ-R⁵, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —C₃-C₈-cycloalkyl, —C₃-C₈-heterocycloalkyl, —(CH₂)ₚ-phenyl, —(CH₂)ₚ—C₅-C₆-heteroaryl, -phenylene-(CH₂)ₚ—R⁵, —NR⁵R⁶, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —(CH₂)ₚ—NR⁵C(O)NR⁶R⁷, —(CH₂)ₚ—S(O)₂R⁵, —(CH₂)ₚ—NR⁵S(O)₂R⁶, —(CH₂)ₚ—S(O)₂NR⁵R⁶, —(CH₂)ₚ—C(O)NR⁵R⁶, —(CH₂)ₚ—NR⁵C(O)R⁶, —(CH₂)ₚ—CR⁴(OH)—R⁵, —O—(CH₂)ₚ—CR⁴(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein phenylene, —C₃-C₈-cycloalkyl, —C₃-C₈-heterocycloalkyl, —(CH₂)ₚ-phenyl and —(CH₂)ₚ—C₅-C₁₀-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—;
R⁴ is hydrogen or —C₁-C₈-alkyl;
R⁵ R⁶
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —C₃-C₈-cycloalkyl, —C₃-C₈-heterocycloalkyl, —C₁-C₆-hydroxyalkyl, —C₁-C₆-haloalkyl, —C₁-C₆-haloalkoxy, —NR⁹R¹⁰, —NR⁹C(O)NR¹⁰, —S(O)₂(C₁-C₆-alkyl), —S(O)₂-phenyl, —NH—S(O)₂(C₁-C₆-alkyl), —NH—S(O)₂-phenyl, —S(O)₂—NH—(C₁-C₆-alkyl), —S(O)₂—NH-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₆-heteroaryl, -phenylene—(CH₂)ₚ—R⁹, wherein phenylene, —C₃-C₈-cycloalkyl, —C₃-C₈-heterocycloalkyl, phenyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 2- to 8-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—;
R⁹, R¹⁰ are independently from each other hydrogen or —C₁-C₈-alkyl;
p is an integer of 0, 1, 2, 3, 4, 5, or 6.

More preferred are compounds of the Formula I of the present invention wherein:
A is -meta-phenylene-;
X is an unsubstituted 4- to 5-membered alkylene tether;
Y is —NR⁴— or —O—;
R¹ and R² are the same or different and are independently from each other selected from the group comprising, preferably consisting of, hydrogen, hydroxy, halogen, nitro, cyano, —(CH₂)ₚ—NR⁵R⁶, —(CH₂)ₚ—NHC(O)R⁶, —(CH₂)ₚ—NHS(O)₂R⁶, —(CH₂)ₚ—NHC(O)NR⁶R⁷, —(CH₂)ₚ—NHC(O)OR⁶, —(CH₂)ₚ—C(O)R⁵, —(CH₂)ₚ—CH(OH)—R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —(CH₂)ₚ—OR⁵, —CR⁵(OH)—R⁶, —O—(CH₂)ₚ—C(O)R⁵, —O—(CH₂)ₚ—CH(OH)—R⁵ and moieties being selected from the group comprising, preferably consisting of, —C₁-C₈-alkyl, —(CH₂)ₚ-phenyl, —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl;
R³ is selected from the group comprising, preferably consisting of, halogen, cyano, amino, benzodioxolyl, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ-C₅-C₁₀-heteroaryl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ-C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ—phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—;
R⁴ is hydrogen or —C₁-C₈-alkyl;
⁵, R⁶,
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, amino, cyano, —C₁-C₈-alkyl, —C₃-C₈-cycloalkyl, —C₁-C₆-hydroxyalkyl, —C₁-C₆-haloalkyl, —C₁-C₆-haloalkoxy, —NR⁹R¹⁰, —NR⁹C(O)NR¹⁰, —S(O)₂(C₁-C₆-alkyl), —S(O)₂-phenyl, —NH—S(O)₂(C₁-C₆-alkyl), —NH—S(O)₂-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₆-heteroaryl, wherein —C₃-C₈-cycloalkyl, phenyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 3- to 7-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—;
R⁹, R¹⁰ are independently from each other hydrogen or —C₁-C₈-alkyl;

p is an integer of 0, 1, 2, 3, or 4.

Furthermore, compounds of the Formula I of the present invention are preferred, wherein:
R⁴ is hydrogen, methyl, or ethyl.

Furthermore, compounds of the present invention are preferred, wherein
R⁵, R⁶,
R⁷ and R⁸ are the same or different and are independently from each other selected from the group comprising, preferably consisting of, hydrogen and moieties, said moieties being selected from the group comprising, preferably consisting of, —C₃-C₈-cycloalkyl, —C₃-C₈-heterocycloalkyl, —C₁-C₈-alkyl, phenyl and —C₅-C₆-heteroaryl, wherein said moieties are unsubstituted or singly or multiply substituted independently from each other with hydroxy, halogen, —C₁-C₆-alkoxy, amino, cyano, —C₁-C₆-alkyl, —C₁-C₆-hydroxyalkyl, —C₁-C₆-haloalkyl, —C₁-C₆-haloalkoxy, —NR⁹R¹⁰, —C(O)NR⁹R¹⁰, —C(O)R⁹, or two members selected from the group comprising, preferably consisting of, R⁵, R⁶, R⁷ and R⁸ form a 3- to 7-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—.

Furthermore, compounds of the Formula I of the present invention are preferred, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷, —(CH₂)ₚNHC(O)OR⁶, —(CH₂)ₚ—C(O)R⁵, —(CH₂)ₚ—CH(OH)—R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —CR⁵(OH)—R⁶, —O—(CH₂)ₚ—C(O)R⁵, —O—(CH₂)ₚ—CH(OH)—R⁵.

Herein compounds are more preferred, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷, —(CH₂)ₚ—NHC(O)OR⁶, —(CH₂)ₚ—C(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—C(O)NR⁵R⁶, —O—(CH₂)ₚ—C(O)R⁵.

More preferred are compounds, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷.

Furthermore compounds of the Formula I of the present invention are preferred, wherein:
R¹ and R² are the same or different and are independently from each other selected from the group comprising, preferably consisting of, —C₁-C₈-alkyl, —(CH₂)ₚ-phenyl, —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl.

Furthermore compounds of the Formula I of the present invention are preferred, wherein:
R³ is selected from the group comprising, preferably consisting of, halogen, cyano, amino, benzodioxolyl, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷.

Herein compounds are more preferred, wherein:
R³ is selected from the group comprising, preferably consisting of, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷; or
R³ is selected from the group comprising, preferably consisting of, halogen, cyano, amino; or
R³ is benzodioxolyl.

Furthermore compounds of the Formula I of the present invention are preferred, wherein:
R³ is selected from the group comprising, preferably consisting of, —C₁-C₈-alkyl, —C₂-C₈-alkenyl, and —C₂-C₈-alkynyl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

Furthermore compounds are more preferred, wherein:
R³ is selected from the group comprising, preferably consisting of, —C₁-C₈-alkyl and —C₂-C₈-alkynyl, wherein said moieties are unsubstituted or singly or multiply substituted independently from each other with halogen, —C₁-C₆-alkoxy, C₁-C₆-alkyl, —(CH₂)ₚ-phenyl, —(CH₂)ₚ—C₅-C₆-heteroaryl, —NR⁵R⁶, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —(CH₂)ₚ—C(O)R⁵, wherein —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

Furthermore compounds of the Formula I of the present invention are preferred, wherein:
R³ is selected from the group comprising, preferably consisting of, —C₃-C₈-cycloalkyl and —C₃-C₈-heterocycloalkyl, and is unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ—phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

Furthermore compounds are more preferred, wherein:
R³ is selected from the group comprising, preferably consisting of, cyclopentyl, cyclohexyl, piperazinyl, piperidinyl, 1,4-diazepanyl and pyrrolidinyl, wherein said moieties are unsubstituted or singly or multiply substituted independently from each other with hydroxy, —C₁-C₆-alkoxy, cyano, —C₁-C₆-alkyl, —C₃-C₈-cycloalkyl, —C₃-C₈-heterocycloalkyl, —NR⁵R⁶, —C₁-C₆-hydroxyalkyl, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

Furthermore compounds are preferred, wherein:
R³ is selected from the group comprising, preferably consisting of, piperazinyl, piperidinyl, and pyrrolidinyl, wherein said moieties are unsubstituted or singly or multiply substituted independently from each other with —C₁-C₆-alkyl, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

Furthermore compounds are more preferred, wherein:
R³ is piperazinyl and is unsubstituted or singly or multiply substituted independently from each other with —C₁-C₆-alkyl, —(CH₂)ₚ—S(O)₂R⁵, —(CH₂)ₚ—C(O)NR⁵R⁶, —(CH₂)ₚ—CH(OH)—R⁵, (CH₂)ₚ—C(O)R⁵.

Furthermore compounds of the Formula I of the present invention are preferred, wherein:
R³ is selected from the group comprising, preferably consisting of, —(CH₂)ₚ-phenyl and —(CH₂)ₚ—C₅-C₁₀-heteroaryl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ—phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

Furthermore compounds are more preferred, wherein:
R³ is selected from the group comprising, preferably consisting of, —(CH₂)ₚ-phenyl and —(CH₂)ₚ—C₅-C₆-heteroaryl, wherein said moieties are unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

Furthermore compounds are preferred, wherein:
R³ is selected from the group comprising, preferably consisting of, phenyl and —C₅-C₆-heteroaryl, wherein said moieties are unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

Furthermore compounds are more preferred, wherein:
R³ is phenyl and is unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—NHS(O)₂R⁶, —(CH₂)ₚ—C(O)NR⁵R⁶, —(CH₂)ₚ—NHC(O)R⁶, —(CH₂)ₚ—C(O)R⁵, —O—(CH₂)ₚ—C(O)R⁵.

Compounds of formula (I) are even more preferred in which
R¹, R² is hydrogen, —N₂, —NH₂, —NHC(O)NR⁵R⁶, —NHC(O)R⁵;
X is an unsubstituted 4-membered tether;
Y is —NH—; and
R³ is a phenyl group which is optionally substituted one or more times with, independently of each other, Hal , C₁-C₆-alkyl , C₁-C₆-alkoxy, —NHC(O)NR⁵R⁶, —NHS(O)₂R⁵, or —NHC(O)R⁵.

More particularly preferred still are the following compounds:

(RS)-1⁵-Bromo-4-imino-4-thia-2,9-diaza-1(2,4)-pyrimidina-3(1,3)-benzenacyclononaphane-4-oxide;

(RS)-1⁵-lodo-4-imino-4-thia-2,9-diaza-1(2,4)-pyrimidina-3(1,3)-benzenacyclononaphane-4-oxide;

(RS)-4-Imino-1⁵-(4-methoxyphenyl)-4-thia-2,9-diaza-1(2,4)-pyrimidina-3(1,3)-benzenacyclononaphane 4-oxide;

N-{4-[(RS)-4-Imino-4-oxo-4λ⁶-thia-2,9-diaza-1(2,4)-pyrimidina-3(1,3)-benzenacyclononaphan-1⁵-yl]phenyl}-N′-phenyl urea;

N-{4-[(RS)-4-Imino-4-oxo-4λ⁶-thia-2,9-diaza-1(2,4)-pyrimidina-3(1,3)-benzenacyclononaphan-1⁵-yl]phenyl}-N′-[3-(trifluormethyl)phenyl]urea;

N-{4-[(RS)-4-Imino-4-oxo-4?⁶-thia-2,9-diaza-1(2,4)-pyrimidina-3(1,3)-benzenacyclononaphan-1⁵-yl]phenyl}-N′-(3-methylphenyl)urea;

N-(3-Ethylphenyl)-N′-{4-[(RS)-4-imino-4-oxo-4λ⁶-thia-2,9-diaza-1(2,4)-pyrimidina-3(1,3)-benzenacyclononaphan-1⁵-yl]phenyl}urea;

(RS)-1-[2-Fluoro-5-(trifluoromethyl)phenyl]-3-{4-[4-imino-4-oxo-4λ⁶-thia-2,9-diaza-1(2,4)-pyrimidina-3(1,3)-benzenacyclononaphan-1⁵-yl]phenyl}urea;

(RS)-4-Imino-1⁵-(2-methyl-4-methoxyphenyl)-4-thia-2,9-diaza-1(2,4)-pyrimidina-3(1,3)-benzenacyclononaphane 4-oxide;

(RS)-2,3-Dichloro-N-{4-[4-imino-4-oxo-4-thia-2,9-diaza-1(2,4)-pyrimidina-3(1,3)-benzenacyclononaphan-1⁵-yl]phenyl}benzenesulfonamide;

(RS)-4-Imino-1⁵-[4-(1-methylethoxy)phenyl]-4-thia-2,9-diaza-1(2,4)-pyrimidina-3(1,3)-benzenacyclononaphane 4-oxide;

(RS)-1⁵-(4-Ethylphenyl)-4-imino-4-thia-2,9-diaza-1(2,4)-pyrimidina-3(1,3)-benzenacyclononaphane 4-oxide;

(RS)-1⁵-(4-Ethoxyphenyl)-4-imino-4-thia-2,9-diaza-1(2,4)-pyrimidina-3(1,3)-benzenacyclononaphane 4-oxide;

(RS)-1⁵-(3-Fluoro-4-methoxyphenyl)-4-imino-4-thia-2,9-diaza-1(2,4)-pyrimidina-3(1,3)-benzenacyclononaphane 4-oxide;

(RS)-1⁵-(4-Ethoxy-3-fluorophenyl)-4-imino-4-thia-2,9-diaza-1(2,4)-pyrimidina-3(1,3)-benzenacyclononaphane 4-oxide;

(RS)—N-{4-[4-Imino-4-oxo-4-thia-2,9-diaza-1(2,4)-pyrimidina-3(1,3)-benzenacyclononaphan-1⁵-yl]phenyl}-1-phenylcyclopropanecarboxamide;

(RS)-2,3-Dichloro-N-{4-[4-imino-4-oxo-4-thia-2,9-diaza-1(2,4)-pyrimidina-3(1,3)-benzenacyclononaphan-1⁵-yl]but-3-ynyl}benzenesulfonamide;

2-{4-[(RS)-4-Imino-4-oxo-4⁶-thia-2,9-diaza-1(2,4)-pyrimidina-3(1,3)-benzenacyclononaphan-1⁵-yl]phenoxy}-1-(2,4-xylyl)ethan-1-one;

(RS)-4-Imino-1⁵-iodo-3⁵-nitro-4-thia-2,9-diaza-1(2,4)-pyrimidina-3(1,3)-benzenacyclononaphane 4-oxide;

N—[(RS)-4-Imino-1⁵-iodo-4-oxo-4λ⁶-thia-2,9-diaza-1(2,4)-pyrimidina-3(1,3)-benzenacyclononaphan-3⁵-yl]pyrrolidine-1-carboxamide; and

N—[(RS)-1⁵-(4-Ethoxyphenyl)-4-imino-4-oxo-4λ⁶-thia-2,9-diaza-1(2,4)-pyrimidina-3(1,3)-benzenacyclononaphan-3⁵-yl]pyrrolidine-1-carboxamide.

The compounds of the present invention can be used in treating diseases of dysregulated vascular growth or diseases which are accompanied by dysregulated vascular growth. Especially the compounds effectively interfere with angiopoietin and therefore influence Tie2 signalling. Surprisingly the compounds block Tie2 signalling, wherein obviously Tie2 kinase activity is blocked with showing no or very low cell toxicity for cells other than endothelial cells at low concentrations, which is an important advantage over prior art substances. This effect will therefore allow prolonged treatment of patients with the compounds offering good tolerability and high anti-angiogenic efficacy, where persistent angiogenesis plays a pathologic role.

The compounds of the present invention can thus be applied for the treatment of diseases accompanied by neoangiogenesis. This holds principally for all solid tumours, e.g. breast, colon, renal, lung and/or brain tumours and can be extended to a broad range of diseases, where pathologic angiogenesis is persistent. This applies for diseases with inflammatory association, diseases associated with oedema of various forms and diseases associated with stromal proliferation and pathologic stromal reactions broadly. Particularly suited is the treatment for gynaecological diseases where inhibition of angiogenic, inflammatory and stromal processes with pathologic character can be achieved. At the same time the toxic side effects on normal proliferating tissue are low. The treatment is therefore an addition to the existing armament to treat diseases associated with neoangiogenesis.

The compounds of the present invention can be used in particular in therapy, and prevention of tumour growth and metastases especially in solid tumours of all indications and stages with or without pre-treatment if the tumour growth is accompanied with persistent angiogenesis. However it is not restricted to tumour therapy but is also of great value for the treatment of other diseases with dysregulated vascular growth. This includes retinopathy and other angiogenesis dependent diseases of the eye (e.g. cornea transplant rejection, age-related macular degeneration), rheumatoid arthritis, and other inflammatory diseases associated with angiogenesis such as psoriasis, delayed type hypersensitivity, contact dermatitis, asthma, multiple sclerosis, restenosis, pulmonary hypertension, stroke and inflammatory diseases of the bowel, such as Crohn's disease. Such diseases include coronary and peripheral artery disease. It can be applied for disease states such as ascites, oedema, such as brain tumour associated oedema, high altitude trauma, hypoxia induced cerebral oedema, pulmonary oedema and macular oedema or oedema following burns and trauma. Furthermore it is useful for chronic lung disease, adult respiratory distress syndrome. Also for bone resorption and for benign proliferating diseases such as myoma, benign prostate hyperplasia and wound healing for the reduction of scar formation. It is therapeutically valuable for the treatment of diseases, where deposition of fibrin or extracellular matrix is an issue and stroma proliferation is accelerated (e.g. fibrosis, cirrhosis, carpal tunnel syndrome etc). In addition it can be used for the reduction of scar formation during regeneration of damaged nerves, permitting the reconnection of axons. Further uses are endometriosis, pre-eclampsia, postmenopausal bleeding and ovarian hyperstimulation.

A second aspect of the invention is a pharmaceutical composition which contains at least one compound of general Formula I, e.g. in form of a pharmaceutically-acceptable salt, or an in vivo hydrolysable ester of at least one compound of general Formula I, and one or more pharmaceutically-acceptable diluents or carriers. This composition is particularly suited for the treatment of diseases of dysregulated vascular growth or of diseases which are accompanied with dysregulated vascular growth as explained above.

In order that the compounds of the present invention be used as pharmaceutical product the compounds or mixtures thereof are provided in a pharmaceutical composition, which beside the compounds of the present invention for enteral, oral or parenteral application contain suitable pharmaceutically acceptable organic or inorganic inert base material, e.g. purified water, gelatin, rubber arabicum, lactate, starch, magnesium stearate, talcum, vegetable oils, polyalkyleneglycol, etc.

The pharmaceutical composition may be provided in a solid form, e.g. as tablets, dragées, suppositories, capsules or in Liquid form, e.g. as a solution, suspension or emulsion. The pharmaceutical composition may additionally contain auxiliary substances, e.g. preservatives, stabilisers, wetting agents or emulsifiers, salts for adjusting the osmotic pressure or buffers.

For parenteral applications, (including intravenous, subcutaneous, intramuscular, intravascular or infusion), sterile injection solutions or suspensions are preferred, especially aqueous solutions of the compounds in polyhydroxyethoxy containing castor oil.

The pharmaceutical composition may further contain surface active agents, e.g. salts of gallenic acid, phospholipids of animal or vegetable origin, mixtures thereof and liposomes and parts thereof.

For oral application tablets, dragées or capsules with talcum and/or hydrocarbon-containing carriers and binders, e.g. lactose, maize and potato starch, are preferred. Further application in liquid form is possible, for example as juice, which contains sweetener if necessary.

The dosage will necessarily be varied depending upon the route of administration, age, weight of the patient, the kind and severity of the illness being treated and similar factors. The daily dose is in the range of 0.5-1,500 mg. A dose can be administered as unit dose or in part thereof and distributed over the day. Accordingly the optimum dosage may be determined by the practitioner who is treating any particular patient.

Another aspect of the present invention is a method which may be used for preparing the compounds according to the present invention.

The following Table lists the abbreviations used in this paragraph, and in the Examples section. NMR peak forms are stated as they appear in the spectra, possible higher order effects have not been considered.

| Abbreviation | Meaning |
|---|---|
| br | broad |
| d | doublet |
| dd | doublet of doublet |
| DCM | dichloromethane |
| dec. | decomposition |
| DIPEA | N,N-diisopropylethyl amine |
| DMF | N,N-dimethylformamide |
| DMSO | dimethyl sulfoxide |
| eq | equivalent |
| ESI | electrospray ionisation: results for the observed |
| | ions are reported as a mass/charge (m/z) ratio |
| GP | general procedure |
| m | multiplet |
| mc | centred multiplet |
| mp. | melting point in degrees Celsius |
| MS | mass spectrometry |
| NMR | nuclear magnetic resonance spectroscopy: |
| | chemical shifts (δ) are given in ppm. |
| POPd | dihydrogen dichlorobis(di-tert-butyl phosphinito- |
| | κP)palladate(2); CombiPhos Catalysts, Inc. |
| q | quartet |
| s | singlet |
| t | triplet |
| TEA | triethylamine |
| THF | tetrahydrofuran |
| TLC | thin layer chromatography |

If the production of the compounds of general Formula I according to the invention is not described, the latter is carried out analogously to known methods.

The structural determination of the macrocyclic fragrances Muskon and Zibeton by Ruzicka ((a) Ruzicka, L. Helv. Chim. Acta 1926, 9, 715. (b) Ruzicka, L. Helv. Chim. Acta 1926, 9, 249) in 1926 marks the beginning of the chemistry of macrocyclic compounds.

In general, medium (8- to 11-membered rings, expressed as C₈-C₁₁ according to the definition above) and large (≧12-membered rings) ring systems are referred to as macrocyclic compounds of general Formula I of the present invention. The established processes for synthesis of macrocyclic compounds are partially based on ring enlargement reactions (Hesse, M. Ring Enlargement in Organic Chemistry, VCH, Weinheim, 1991), and more rarely on ring contractions (Hayashi, T. J. Org. Chem. 1984, 49, 2326).

The most frequently used method is the cyclisation of bifunctional acyclic precursors (Review articles on the Synthesis of Macrocyclic Compounds: (a) Roxburgh, C. J. Tetrahedron 1995, 51, 9767. (b) Meng, Q. Top. Curr. Chem. 1991, 161, 107. (c) Paterson, I. Tetrahedron 1985, 41, 3569. (d) Masamune, S. Angew. Chem. 1977, 89, 602. (e) Nicolaou, K. C. Tetrahedron 1977, 33, 683).

The reactions outlined below are performed in the presence of a suitable solvent, for example, simple ketones, such as acetone; alcohols, such as, e.g., ethanol or butanol; esters, such as, for example, ethyl acetate; aromatic solvents, such as, for example, toluene or benzene; halogenated or halogen-free hydrocarbons such as hexane, dichloromethane, dichloroethane, or chloroform; ethers such as diethyl ether, tetrahydrofurane, 1,4-dioxane, or anisol as well as polar aprotic solvents, such as acetonitrile, DMSO, DMF or N-methylpyrrolidone, or mixtures of these solvents, also with the addition of water.

Suitable reducing agents are, for example, TiCl₃ and SnCl₂.

Certain steps, such as the formation of the macrocycles of the Formula I from their acyclic precursors, may require the presence of a suitable acid, for example inorganic acids such as hydrogen chloride, hydrogen bromide, sulfuric acid or BBr₃; organic acids such as acetic acid, formic acid, or trifluoroacetic acid; metal salts such as TiCl₃, SnCl₂, Ln(OTf)₃, etc.

Certain steps may furthermore require the presence of a suitable base, which may be an amine, such as triethylamine, diisopropylethylamine, or pyridine, or an inorganic base, such as sodium hydride, potassium hydride, potassium carbonate, potassium phosphate, or caesium carbonate, or an alkoxide, such as sodium methoxide, sodium ethoxide, sodium tert-butoxide, or potassium tert-butoxide, or an organic amide base, such as lithium diisopropyl amide or lithium hexamethyldisilylamide, or an organometallic base, such as butyllithium.

Therein a method of preparing a compound of the present invention according to the general Formula I:

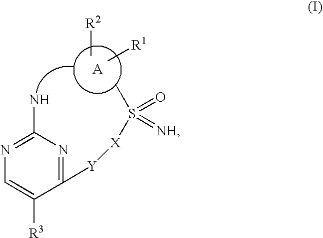

in which A, X, Y, R¹, R², and R³ have the meaning as given herein above, comprises the following general steps:
A) A disulfide of Formula A is transferred into a sulfoximine of Formula B 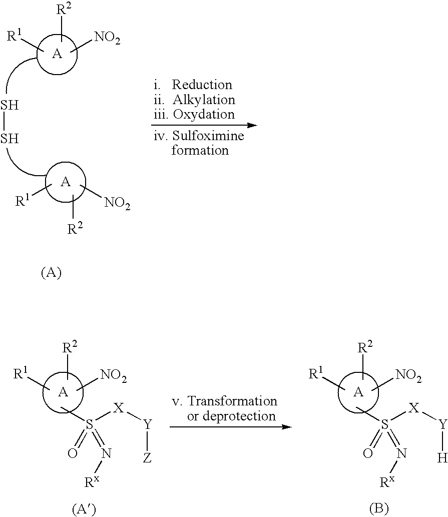
i. Reduction to the free thiol with a suitable reducing agent, ii.

Alkylation with e.g. Z-Y—X-Halogen, iii. Oxidation to give the sulfoxide, iv. Transformation into the sulfoximine e.g. with NaN₃/H₂SO₄/CHCl₃; v. Transfomation or deprotection to give B.

in which A, Y, R¹ and R² have the meaning as given herein above, Z is a protecting or activating group suitable e.g. to protect Y from oxidation or substitution, or to prepare Y for further reaction, and in which R^{x} is, for example, selected from the group comprising, preferably consisting of, hydrogen, —C(O)OR⁵, —C(═O)R⁵ , C(═O)NR⁵R⁶, —S(O)₂R⁵, and —S(O)₂(CH₂)ₚ—Si(C₁-C₄-alkyl)₃, in which p, R⁵ and R⁶ have the same meaning as herein above.

Examples of said residues Z, which are well-known and merely illustrating, but not limiting the invention, may e.g. comprise a phthalimido or tert-butoxycarbonyl moiety (resulting in compounds of the Formula I in which Y stands for —NH—) or a silyl ether such as tert-butyl dimethyl silyloxy or tert-butyl diphenylsilyloxy (resulting in compounds of the Formula I in which Y stands for —O—). Suitable deprotection reactions to transfer the intermediate Y-Z into free Y—H comprise e.g. the hydrazinolysis of a phthalimido moiety, acidic cleavage of a tert-butoxycarbonyl group, or the cleavage of a silyl ether by suitable reagents such as tetrabutyl ammonium fluoride. As an additional example, Y-Z could also refer to a suitable leaving group, such as a mesylate or a tosylate, which is then converted into a thioester by treatment with e.g. potassium thioacetate or potassium thiobenzoate. The free thiol (in which Y stands for —S—) can be generated by subsequent saponification with e.g. aqueous sodium hydroxide.

Further suitable protecting groups and procedures for their introduction and cleavage are well known to the person skilled in the art; particular reference is made to T. W. Greene and P. G. M. Wuts, Protective Groups in Organic Synthesis, John Wiley & Sons (1999).

The sulfoximino group (—S(═O)(═NH)—) can be generated, e.g. from the corresponding sulfoxide either in free or substituted form (for a review article see e.g. M. Reggelin, C. Zur, Synthesis 2000, 1). Alternatively, the sulfoximine can be substituted on the NH group in a separate subsequent step. The resulting compounds featuring a substituted sulfoximino group —S(═O)(═NR^{x})— in which R^{x} is selected from the group comprising, preferably consisting of, hydrogen, —C(O)OR⁵, —C(═O)R⁵ , —COCHNR⁵R⁶, C(═O)NR⁵R⁶, —S(O)₂R⁵, and —S(O)₂(CH₂)ₚ—Si(C₁-C₄-alkyl)₃, in which p, R⁵ and R⁶ have the same meaning as given herein above.

Methods for the preparation of N-unsubstituted sulfoximines have been reported in the scientific literature, see e.g. C. R. Johnson, J. Am. Chem. Soc. 1970, 92, 6594; C. R. Johnson et al., J. Org. Chem, 1974, 39, 2458; H. Okamura, C. Bolm, Org. Lett. 2004, 6, 1305; the latter two methods also allow converting non-racemic sulfoxides, which are e.g. available by asymmetric oxidation of thioethers (see, for example, H. Kagan et al., J. Org. Chem. 1995, 60, 8086) into the corresponding sulfoximines without loss of stereochemical information. Two very recent publications describe the preparation of N-Nosyl sulfoximines which can conveniently be further transformed into their N-unsubstituted analogues, see G. Y. Cho, C. Bolm, Tetrahedron Lett. 2005, 46, 8807, and G. Y. Cho, C. Bolm, Org. Lett. 2005, 7, 4983.

The resulting sulfoximines can be subsequently reacted with suitable precursors of R^{x}, hereinafter referred to as R^{x,}, to form the substituted sulfoximines —S(═O)(═NR^{x})—:

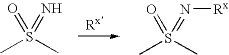

An example of R^{x,}, which is merely illustrating but not limiting the invention is e.g. R^{x,}=Cl·C(═O)—OR⁵, resulting in compounds in which R^{x} is C(O)OR⁵.

Alternatively, there are also methods known which directly lead to N-substituted sulfoximines —S(═O)(═NR^{x})—, see e.g. S. Cren et al., Tetrahedron Lett. 2002, 43, 2749, J. F. K. Mueller and P. Vogt, Tetrahedron Lett. 1998, 39, 4805, T. Bach and C. Korber, Tetrahedron Lett. 1998, 39, 5015.

Such substituted sulfoximino compounds may be useful intermediates in the preparation of compounds of the Formula I as they may prevent the NH group within the sulfoximine moiety from participating in undesired side reactions in the subsequent steps in the preparation of compounds of the Formula I.
B) The sulfoximine of Formula B is transferred into a sulfoximine of Formula C 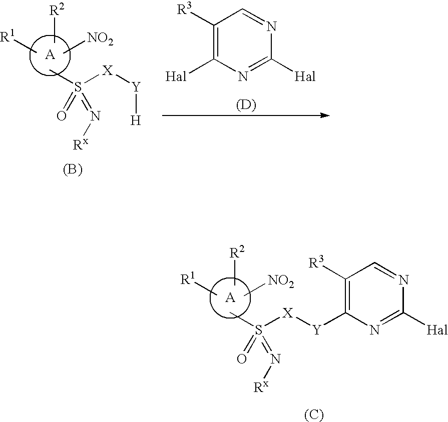
Coupling sulfoximine B with dihalopyrimidine D, e.g. under using a suitable base, e.g. Et₃N or NaH, in a suitable solvent, e.g. acetonitrile or DMF.

Conversion of B into C may be accomplished by coupling B with a dihalopyrimidine D, in which A, X, Y, R¹, R², R³ and R^{x} have the meaning as given herein above, and wherein Hal is halogen, suitable 2,4-dihalopyrimidine building blocks with various R³ are well known in the scientific literature and are partly commercially available. As outlined further below, R³ can also be further elaborated after completion of the macrocycle synthesis.
C) The sulfoximine of Formula C is transferred into a macrocyclic sulfoximine of Formula I 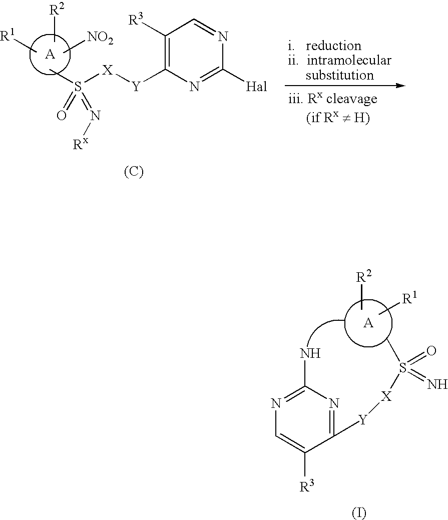
i. Reduction with e.g. TiCl₃ or SnCl₂, ii. Intramolecular substitution e.g. by treatment with HCl/dioxane in aq. acetonitrile, iii. Removal of R^{x} if R^{x} is not hydrogen, e.g. by hydrogenolysis if R^{x}═C(O)O—CH₂Ph.

in which A, X, Y, R¹, R², R³ and R^{x} have the meaning as given herein above, by a sequence of a reduction of the nitro group in (C) with an appropriate agent (i.), followed by intramolecular substitution of a suitable substituent, e.g. Hal, on the pyrimidine-C-2 in (C) by the resulting amino group (ii.) e.g. by reacting with hydrochloric acid in an appropriate solvent at ambient or elevated temperature. Finally, if R^{x} is not hydrogen, cleavage of R^{x}, in step iii, provides a compound of general formula (I).

The sulfoximino-macrocycles of Formula I can thus be prepared, for example, starting from disulfides A which can be transferred into sulfoximines B according to literature procedures (e.g. i. Overman et al., Synthesis 1974 (1), 59; ii. Pasto et al., J. Am. Chem. Soc. 1994, 116, 8978; iii. Kim et al., Synthesis 2002 (17), 2484; iv. C. R. Johnson, J. Am. Chem. Soc. 1970, 92, 6594); for explicit protocols in this document, see e.g. the preparation of intermediates 1 to 4. Methods suitable for the subsequent transformation of sulfoximines B into the macrocycles of the Formula I are described in WO 2004/026881 A; such a sequence is also highlighted by the preparations of intermediates 5 and 8 and example compound 1.2 in this document.

The person skilled in the art will readily recognise the possibility of various interconversions of R¹ and/or R² during A), B) and/or C). Such interconversions may be exemplified, but are not limited to, reduction of a nitro group to an amine, followed by acylation, sulfonylation, or urea/carbamate formation, or by nuclophilic displacement of an halide or a nitro group, e.g. by an alkoxide, a phenolate, or a thiolate.

Furthermore, macrocycles of the Formula I according to the present invention (wherein R³ is shown as bromine/iodine) may subsequently be further elaborated by modification of the R³ position to obtain other compounds according to the present invention in view of R³ position, for example by transition metal, e.g. palladium and/or copper catalysed coupling reactions such as Suzuki, Heck, Stille or Sonogashira couplings, or further by amination methods if R³ is a halogen, preferably Br or I at the beginning of the reaction. Such aminations are well known to those skilled in the art and are widely described in the scientific literature; see e.g. J. C. Antilla, J. M. Baskin; T. E. Barder, S. L. Buchwald, J. Org. Chem. 2004, 69, 5578; T. A. Jensen, X. Liang, D. Tanner, N. Skjaerbaek, J. Org. Chem. 2004, 69, 4936; R. Varala, E. Ramu, M. M. Alam, S. R. Adapa, Synlett 2004, 10, 1737; C. Meyers, B. U. W. Maes, K. T. J. Loones, G. Bat, G. L. F. Lemière, R. A. Dommisse, J. Org. Chem. 2004, 69, 6010; H. Zhang, Q Cai, D Ma, J. Org. Chem. 2005, 70, 5164; EP 0103464 B1; or references cited therein. Similar introduction of an aryloxy or heteroaryloxy moiety is also possible, see e.g. E. J. Reinhard et at., J. Med. Chem. 2003, 46, 2152, and references cited therein.

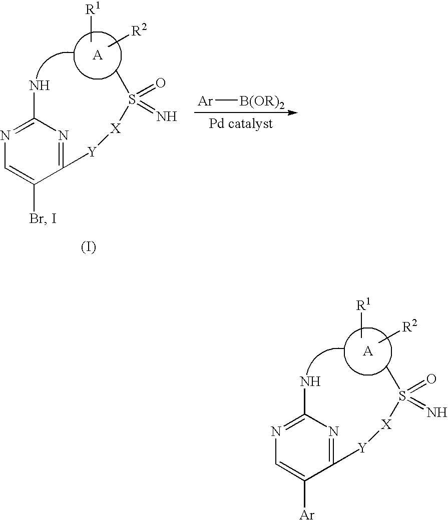

Scheme 2: Suzuki coupling of macrocycles of Formula I, wherein Ar means aryl or heteroaryl with the same meaning as in Formula I for R³, and wherein B(OR)₂ refers to a boronic acid or an ester thereof.

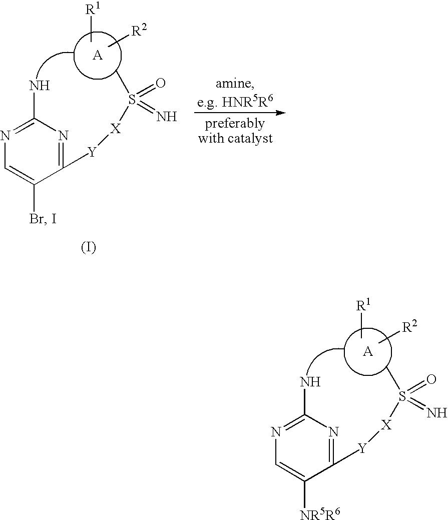

Scheme 3: Amination of macrocycles of Formula I wherein R³ is bromine or iodine.

Appropriate coupling partners are either commercially available or can be prepared by simple standard functionalization procedures as shown in Scheme 4 well known to those skilled in the art.

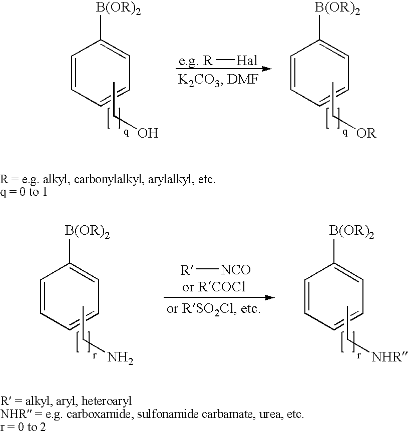

Scheme 4: Synthesis of building blocks for Suzuki couplings wherein B(OR)₂ refers to a boronic acid or an ester thereof

Additional methods suitable to prepare the compounds of the invention are well known and/or readily accessible to those skilled in the art and are described in the scientific literature, e.g. monographs such as R. Larock, Comprehensive Organic Transformations, VCH Publishers (1999), T. W. Greene and P. G. M. Wuts, Protective Groups in Organic Synthesis, John Wiley & Sons (1999), and L. Paquette, ed., Encyclopedia of Reagents for Organic Synthesis, John Wiley & Sons (1995), and in the sources cited in this document.

### EXAMPLE COMPOUNDS

The following examples illustrate the preparation of the compounds of the invention without limiting the scope of the claims.

General Procedures

GP 1: Reaction of Aminophenylboronic Acid Esters with Isocyanates

A solution of the respective aminophenylboronic acid ester in DCM (5 mL per mmol boronic ester) was treated with the respective isocyanate (1.05 eq.), followed by TEA (1.1 eq.) at room temperature under an atmosphere of nitrogen. The resulting mixture was stirred overnight and then analysed by TLC. If the reaction did not reach completion after 20 h, additional reagents (isocyanate, 0.26 eq.; and TEA, 0.28 eq.) were supplemented and stirring was continued until the reaction was complete according to TLC. The mixture was evaporated and then subjected to column chromatography. In some cases, the intermediates obtained were still impure and used without further purification.

Typically, the reactions were run on a 0.5 to 1 mmol scale.

GP 2: Suzuki Coupling

A solution of the respective macrocyclic halide in DMF (8 mL per mmol halide) was treated with the respective organoboron compound (1.25 eq.), K₂CO₃ (2.5 eq., either as a solid or as 2 M aqueous solution), and POPd (2.5 mol- %) at room temperature. The stirred resulting mixture was heated to 100° C. The reaction was monitored by TLC, and additional portions of POPd (2.5 mol- %) and if consumed by then organoboron compound (1.25 eq.) were added. Stirring was continued for another 2 h, and addition of reagents, followed by 2 h stirring at 100° C. was repeated until the macrocyclic halide was completely consumed. After cooling to room temperature, water was added and the resulting suspension was stirred for 30 min. The crude product was isolated by vacuum filtration, dried in vacuo, and purified by column chromatography, followed by trituration with methanol, and/or preparative HPLC (e.g. YMC Pro C18RS 5 μ, 150×20 mm, 0.2% NH₃ in water/acetonitrite) to give the pure coupling products.

Typically, the reactions were run on a 0.25 mmol scale.

PREPARATION OF INTERMEDIATES

### Intermediate 1

### Preparation of 2-[4-(3-Nitro-phenylsulfanyl)-butyl]-isoindole-1,3-dione

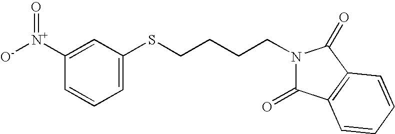

Triphenylphosphine (6.26 g, 23.8 mmol) was added to a solution of bis-(3-nitrophenyl)-disulfide (5.00 g, 16.2 mmol) in dioxane (65 mL) and water (16.3 mL), and the resulting mixture was stirred overnight at room temperature. The solvent was removed in vacuo, toluene was added, and the mixture was evaporated again. The residue was dissolved in ethanol (48. 5 mL) and treated with sodium hydroxide (850 mg, 21.3 mmol). To this mixture, 2-(4-bromo-butyl)-isoindole-1,3-dione (8.52 g, 30.2 mmol) was added, followed by stirring overnight at room temperature. The reaction mixture was poured into water and was then extracted with ethyl acetate (3×). The combined organic layers were dried (Na₂SO₄), filtered and evaporated. The crude residue was purified by column chromatography (hexane/ethyl acetate 1:1) to give the desired product (7.10 g, 19.9 mmol, 61% yield).

¹H-NMR (DMSO): 7.99 (m, 1H), 7.92 (m, 1H), 7.82 (s, 4H), 7.70 (m, 1H), 7.53 (m, 1H), 3.59 (t, 2H), 3.13 (t, 2H), 1.78 (m, 2H), 1.62 (m, 2H).

### Intermediate 2

### Preparation of 2-[4-(3-Nitro-benzolsulfinyl)-butyl]-isoindole-1,3-dione

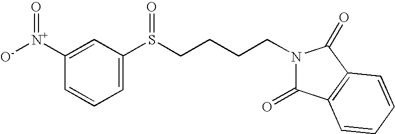

A solution of 2-[4-(3-nitro-phenylsulfanyl)-butyl]-isoindole-1,3-dione (3.90 g, 10.9 mmol) in acetonitrile (39 mL) was treated with anhydrous FeCl₃ (51 mg, 0.31 mmol) and periodic acid (2.65 g, 11.6 mmol). The resulting mixture was stirred for approx. 30 min at ambient temperature and then slowly poured into a solution of sodium thiosulfate (11.1 g) in ice water (75 mL). The mixture was left for another 30 min and was then extracted with DCM (2×). The combined organic Layers were washed with dilute brine, dried (Na₂SO₄), filtered, and concentrated to give the title compound (4.14 g, 10.9 mmol, quantitative yield).

¹H-NMR (DMSO): 8.44 (m, 1H), 8.32 (m, 1H), 8.04 (m, 1H), 7.82 (m, 5H), 3.54 (t, 2H), 3.15 (m, 1H), 2.89 (m, 1H), 1.70 (m, 4H).

MS (ESI):[M+H]⁺=373.

### Intermediate 3

### Preparation of (RS)—S-(3-Nitrophenyl)-S-[4-(1,3-dioxo-2,3-dihydro-1H-isoindol-2-yl)butyl]sulfoximide

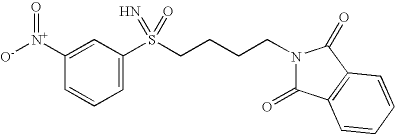

Concentrated sulphuric acid (2.8 mL) was added dropwise to a suspension of 2-[4-(3-nitro-benzolsulfinyl)-butyl]-isoindole-1,3-dione (2.07 g, 5.45 mmol) and sodium azide (795 mg, 12.2 mmol) in CHCl₃ (11.1 mL) under ice cooling. The resulting mixture was stirred at 45° C. for 25 h before being cooled to ambient temperature. Subsequently, ice-cooled diluted aqueous NaOH was added until an alkaline pH was maintained. The mixture was extracted with ethyl acetate (3×), and the combined organic layers were washed with brine, dried (Na₂SO₄), filtered and evaporated. The crude residue was purified by column chromatography (ethyl acetate) to give the desired product (1.30 g, 3.36 mmol, 62% yield).

¹H-NMR (DMSO): 8.58 (m, 1H), 8.46 (m, 1H), 8.27 (m, 1H), 7.85 (m, 5H), 4.62 (s, 1H), 3.51 (t, 2H), 3.29 (m, 2H), 1.60 (m, 4H).

### Intermediate 4

### Preparation of (RS)—S-(4-Aminobutyl)-S-(3-nitrophenyl)sulfoximide

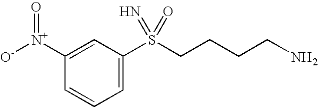

A solution of (RS)—S-(3-nitrophenyl)-S-[4-(1,3-dioxo-2,3-dihydro-1H-isoindol-2-yl)butyl]sulfoximide (1.29 g, 3.3 mmol) in ethanol (142 mL) was treated with hydrazine hydrate (3.4 mL) and heated under reflux for 6 h. Upon standing overnight at room temperature, a precipitate was formed which was isolated by vacuum filtration, and the filtrate was evaporated to give the title compound (920 mg, 3.3 mmol, quantitative yield).

¹H-NMR (DMSO): 8.60 (m, 1H), 8.49 (m, 1H), 8.30 (m, 1H), 7.90 (m, 1H), 4.59 (br, 1H), 3.30 (m, 2H), 2.46 (m, 2H), 1.53 (m, 2H), 1.31 (m, 2H).

### Intermediate 5

### Preparation of (RS)—S-{4-[(5-lodo-2-chloropyrimidin-4-yl)amino]butyl}-S-(3-nitrophenyl)sulfoximide

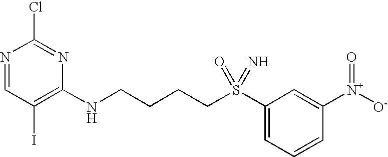

A solution of 2,4-dichloro-5-iodo-pyrimidine (586 mg, 2.14 mmol) in acetonitrile (2 mL) was added to a solution of (RS)—S-(4-aminobutyl)-S-(3-nitrophenyl)sulfoximide (550 mg, 2.14 mmol) in acetonitrile (8 mL) at room temperature. Triethylamine (0.30 mL) was added, and the resulting mixture was stirred for 23 h. The solvent was evaporated and the crude residue was purified by column chromatography (DCM/EtOH 95:5) to give the title compound (690 mg, 1.40 mmol, 65% yield).

¹H-NMR (DMSO): 8.59 (m, 1H), 8.49 (m, 1H), 8.26 (m, 2H), 7.88 (m, 1H), 7.32 (m, 1H), 4.60 (s, 1H), 3.30 (m, 4H), 1.54 (m, 4H).

MS (ESI):[M+H]⁺=496.

### Intermediate 6

### Preparation of (RS)—S-{4-[(5-Bromo-2-chloropyrimidin-4-yl)amino]butyl}-S-(3-nitrophenyl)sulfoximide

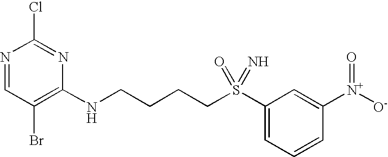

Triethylamine (0.27 mL) was added to a solution of (RS)—S-(4-aminobutyl)-S-(3-nitrophenyl)sulfoximide (200 mg, 0.78 mmol) and 5-bromo-2,4-dichloro-pyrimidine (177 mg, 0.78 mmol) in acetonitrile (3.5 mL), followed by stirring overnight at room temperature. The mixture was poured into brine and was then extracted with ethyl acetate (3×). The combined organic layers were dried (Na₂SO₄), filtered and evaporated. The crude residue was purified by column chromatography (ethyl acetate) to give the desired product (245 mg, 0.55 mmol, 70% yield).

¹H-NMR (DMSO): 8.58 (m, 1H), 8.49 (m, 1H), 8.28 (m, 1H), 8.19 (s, 1H), 7.88 (m, 1H), 7.70 (t, 1H), 4.61 (s, 1H), 3.30 (m, 4H), 1.53 (m, 4H).

### Intermediate 7

### Preparation of (RS)—S-{4-[(5-Bromo-2-chloropyrimidin-4-yl)amino]butyl}-N-(ethoxycarbonyl)-S-(3-nitrophenyl)sulfoximide

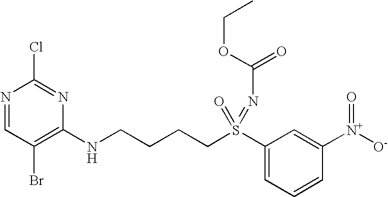

A solution of (RS)—S-{4-[(5-bromo-2-chloropyrimidin-4-yl)amino]butyl}-S-(3-nitrophenyl)sulfoximide (240 mg, 0.54 mmol) in pyridine (5 mL) was treated dropwise with ethyl chloroformiate (0.24 mL, 2.49 mmol) at ambient temperature. The resulting mixture was stirred for 5 h and was then poured into diluted brine. After extraction with ethyl acetate, the combined organic layers were dried (Na₂SO₄), filtered and evaporated to give the title compound (260 mg, 0.50 mmol, 93% yield).

¹H-NMR (DMSO): 8.57 (m, 2H), 8.31 (m, 1H), 8.19 (s, 1H), 7.96 (m, 1H), 7.72 (t, 1H), 3.91 (m, 2H), 3.75 (m, 2H), 3.30 (m, 2H), 1.61 (m, 4H), 1.07 (t, 3H).

### Intermediate 8

### Preparation of (RS)—S-(3-Aminophenyl)-S-{4-[(5-iodo-2-chloropyrimidin-4-yl)amino]butyl}sulfoximide

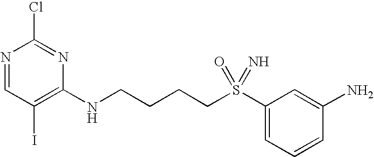

A 10% solution of TiCl₃ in approx. 20-30% hydrochloric acid (12 mL, Aldrich) was added to a solution of (RS)—S-{4-[(5-iodo-2-chloropyrimidin-4-yl)amino]butyl}-S-(3-nitrophenyl)sulfoximide (550 mg, 1.11 mmol) in THF (50 mL) at ambient temperature. After stirring for 30 min, another portion of 10% solution of TiCl₃ in ˜20-30% hydrochloric acid (1.5 mL) was added, and stirring at room temperature was continued for 30 min. Subsequently, the mixture was poured into aqueous 1 M NaOH containing crushed ice. The mixture was extracted with ethyl acetate (3×). The combined organic layers were dried (Na₂SO₄), filtered and evaporated. The crude residue was purified by column chromatography (DCM/EtOH 9:1) to give the desired product (300 mg, 0.65 mmol, 58% yield).

MS (ESI):[M+H]⁺=466.

### Intermediate 9

### Preparation of (RS)—S-(3-Aminophenyl)-S-{4-[(5-bromo-2-chloropyrimidin-4-yl)amino]butyl}sulfoximide

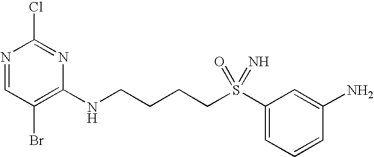

A 10% solution of TiCl₃ in approx. 20-30% hydrochloric acid (1.7 mL, Aldrich) was added to a solution of (RS)—S-{4-[(5-bromo-2-chloropyrimidin-4-yl)amino]butyl}-S-(3-nitrophenyl)sulfoximide (55 mg, 0.12 mmol) in THF (2.5 mL) at ambient temperature. The resulting mixture was stirred for 5 h at ambient temperature and was then poured into a mixture of aqueous 1 M NaOH and crushed ice. After saturation with NaCl, the mixture was extracted with ethyl acetate (3×). The combined organic layers were dried (Na₂SO₄), filtered and evaporated to give the title compound (48 mg, 0.11 mmol, 93% yield).

¹H-NMR (DMSO): 8.21 (s, 1H), 7.71 (t, 1H), 7.15 (m, 1H), 7.07 (m, 1H), 6.93 (m, 1H), 6.74 (m, 1H), 5.53 (s, 2H), 3.88 (s, 1H), 3.30 (m, 2H), 3.05 (m, 2H), 1.53 (m, 4H).

### Intermediate 10

### Preparation of (RS)—S-(3-Aminophenyl)-S-{4-[(5-bromo-2-chloropyrimidin-4-yl)amino]butyl}-N-(ethoxycarbonyl)sulfoximide

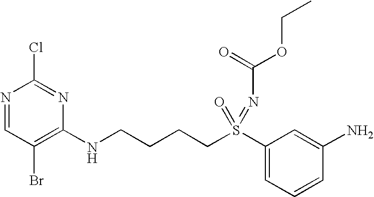

A 10% solution of TiCl₃ in approx. 20-30% hydrochloric acid (6.7 mL, Aldrich) was added to a solution of (RS)—S-{4-[(5-bromo-2-chloropyrimidin-4-yl)amino]butyl}-N-(ethoxycarbonyl)-S-(3-nitrophenyl)sulfoximide (255 mg, 0.49 mmol) in THF (10 mL) at 0° C. under an atmosphere of nitrogen. The resulting mixture was stirred for 6 h at ambient temperature and was then poured into a mixture of aqueous 1 M NaOH and crushed ice. After saturation with NaCl, the mixture was extracted with ethyl acetate (3×). The combined organic layers were dried (Na₂SO₄), filtered and evaporated to give the title compound (179 mg, 0.36 mmol, 74% yield).

¹H-NMR (DMSO): 8.21 (s, 1H), 7.73 (t, 1H), 7.22 (m, 1H), 7.03 (m, 1H), 6.88 (m, 1H), 6.82 (m, 1H), 5.69 (s, 2H), 3.88 (m, 2H), 3.48 (m, 2H), 3.30 (m, 2H), 1.53 (m, 4H), 1.07 (t, 3H).

### Intermediate 11

### Preparation of Ethyl(RS)—N-[1⁵-bromo-4-oxo-4λ⁶-thia-2,9-diaza-1(2,4)-pyrimidina-3(1,3)-benzenacyclononaphan-4-ylidene]carbamate

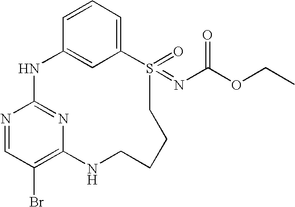

A solution of (RS)—S-(3-aminophenyl)-S-{4-[(5-bromo-2-chloropyrimidin-4-yl)amino]butyl}-N-(ethoxycarbonyl)sulfoximide (160 mg, 0.33 mmol) in acetonitrile (10 mL) was added to a mixture of acetonitrile/water/4 M solution of HCl in 1,4-dioxane (45 mL/5 mL/0.5 mL) at a temperature of 80° C. over a period of 4 h by means of a syringe pump. After 24 h, all volatiles were removed in vacuo and sat. aq. NaHCO₃ solution (50 mL) and ethyl acetate (250 mL) were added. The organic Layer was separated and the aqueous layer was extracted with ethyl acetate (4×). The combined organic layers were washed with brine, dried (Na₂SO₄), filtered and evaporated. The residue was triturated with tert.-butyl methyl ether to give the desired compound (135 mg, 0.30 mmol, 90% yield).

¹H-NMR (DMSO): 9.69 (s, 1H), 8.38 (m, 1H), 8.04 (s, 1H), 7.55 (m, 1H), 7.47 (m, 1H), 7.33 (m, 2H), 3.95 (q, 2H), 3.75 (m, 2H), 3.03 (m, 2H), 1.75 (m, 4H), 1.13 (t, 3H).

MS (ESI):[M+H]⁺=454.

### Intermediate 12

### Preparation of [4-(3,5-dinitro-phenylsulfanyl)-butyl]-carbamic acid tert-butyl ester

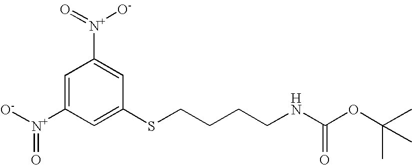

To a solution of 3,5-dinitrobenzenethiol (1.02 g, 5.12 mmol) in ethanol (38 mL) was added powdered sodium hydroxide (246 mg, 6.14 mmol), followed by 4-bromobutyl carbamic acid tert-butyl ester (1.42 g, 5.63 mmol), and the resulting mixture was stirred for 24 h at room temperature. After concentration in vacuo, the residue was dissolved in ethyl acetate (15 mL) and washed with water (3×5 mL). The organic layer was dried over sodium sulphate and concentrated. The residue was purified by chromatography on silica gel to give the pure target compound (1.63 g, 4.39 mmol, 86% yield).

HNMR (DMSO, 300 MHz): 8.50 (t, 1 H); 8.39 (d, 2 H); 6.81 (t br, 1 H); 3.20 (t, 2 H); 2.90 (mc, 2 H); 1.42-1.66 (m, 4 H); 1.32 (s, 9 H).

MS (ESI): [M+H]⁺=372; [M−C₄H₈]⁺=316.

### Intermediate 13

### Preparation of (RS)-[4-(3,5-dinitro-benzenesulfinyl)-butyl]-carbamic acid tert-butyl ester

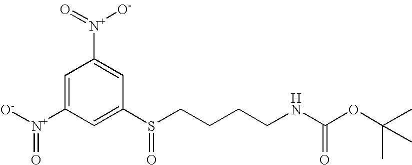

A solution of [4-(3,5-dinitro-phenylsulfanyl)-butyl]-carbamic acid tert-butyl ester (1.98 g, 5.33 mmol) in methanol (100 mL) was added to a solution of sodium periodate (2.39 g; 11.2 mmol) in water (22 mL) at a temperature of 0° C. After stirring at 0° C. for 30 minutes, the reaction mixture was allowed to warm up to room temperature and stirring was continued for 36 h. After filtering off the precipitate, the filtrate was concentrated in vacuo, diluted with water (15 mL), and extracted with DCM (2×50 mL). The combined organic Layers were dried over magnesium sulfate and evaporated, followed by column chromatography on silica get, to give the desired sulfoxide (2.05 g, 5.29 mmol; 99%).

HNMR (DMSO, 400 MHz): 8.87 (t, 1 H); 8.83 (d, 2 H); 6.78 (t br, 1 H); 3.15-3.26 (m, 1 H); 2.80-2.98 (m, 3 H); 1.58-1.72 (m, 1 H); 1.29-1.48 (m, 3H); 1.32 (s, 9 H).

MS (ESI): [M+H]⁺=388; [M−C₄H₈]⁺=332.

### Intermediate 14

### Preparation of (RS)—S-(3,5-dinitrophenyl)-S-{4-[tert-butoxycarbonylamino]-butyl}-N-(trifluoroacetyl)sulfoximide

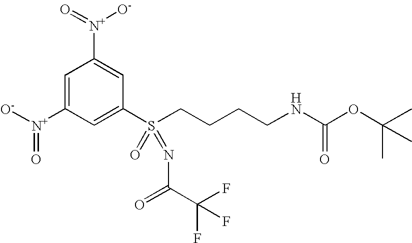

A mixture of (RS)-[4-(3,5-dinitro-benzenesulfinyl)-butyl]-carbamic acid tert-butyl ester (1.66 g, 4.28 mmol), rhodium (II) acetate dimer (95 mg, 5 mol- %), trifluoroacetamide (0.97 g, 8.57 mmol), magnesium oxide (0.69 g, 17.1 mmol), and iodosobenzene diacetate (2.07 g, 6.43 mmol) in DCM (43 mL) was stirred at room temperature for 20 h. The mixture was then filtered off the insolubles and the filtrate was concentrated in vacuo. The residue was purified by column chromatography on silica gel to give the target compound (1.78 g, 3.57 mmol, 83% yield).

HNMR (DMSO, 300 MHz): 9.12 (t, 1 H); 9.00 (d, 2 H); 6.80 (t br, 1 H); 4.03-4.32 (m, 2 H); 2.88 (mc, 2 H); 1.15-1.80 (m, 4 H); 1.29 (s, 9 H).

MS (ESI): [M−H]⁻=497; [M+HCOO]⁻=543.

### Intermediate 15

### Preparation of (RS)—S-(3,5-dinitrophenyl)-S-{4-[tert-butoxycarbonylamino]-butyl}-sulfoximide

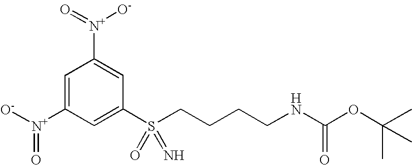

To a suspension of (RS)—S-(3,5-dinitrophenyl)-S-{4-[(tert-butoxycarbonylamino]butyl}-N-(trifluoro-acetyl)sulfoximide (1.75 g, 3.51 mmol) in methanol (35 mL) and DCM (35 mL) was added potassium carbonate (243 mg, 1.76 mmol) and the resulting mixture was first stirred fo 2 h at room temperature and was then left at 4° C. overnight. Water (50 mL) was added, and the resulting mixture was extracted with DCM (3×100 mL). The combined organic layers were dried over magnesium sulfate and evaporated to give the desired free sulfoximine (1.20 g, 2.98 mmol, 85%) which was used without further purification.

HNMR (DMSO, 300 MHz): 9.01 (t, 1 H); 8.88 (d, 2 H); 6.74 (t br, 1 H); 4.92 (s br, 1 H); 3.30-3.42 (m, 2 H); 2.83 (mc, 2 H); 1.43-1.59 (m, 2 H); 1.26-1.42 (m, 2 H); 1.29 (s, 9 H).

MS (ESI): [M+H]⁺=403; [M−C₄H₈]⁺=347.

### Intermediate 16

### Preparation of (RS)—S-(3,5-dinitrophenyl)-S-{4-[(5-iodo-2-chloropyrimidin-4-yl)amino]butyl}sulfoximide

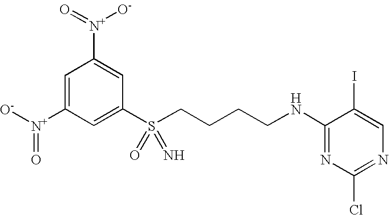

To a solution of (RS)—S-(3,5-dinitrophenyl)-S-{4-[(tert-butoxycarbonylamino]butyl}-sulfoximide (1.06 g, 2.63 mmol) in DCM (13 mL) was added a 4 N solution of hydrogen chloride in 1,4-dioxane (6.59 mL, 26.3 mmol HCl) and the resulting mixture was stirred at room temperature for 90 minutes. The precipitate formed during the reaction was filtered, washed with DCM and dried in vacuo. The resulting solid was then dissolved in acetonitrile (10.5 mL), and TEA (1.18 mL, 3.25 mmol) was added at room temperature, followed by addition of a solution of 2,4-dichloro-5-iodopyrimidine (718 mg, 2.61 mmol) in acetonitrile (2.7 mL). The resulting mixture was then stirred for 2.5 h at room temperature. After concentration in vacuo, the crude residue was purified by column chromatography on silica gel to give the target compound (816 mg, 1.51 mmol; 57% yield).

HNMR (DMSO, 400 MHz): 8.99 (t, 1 H); 8.86 (d, 2 H); 8.23 (s, 1 H); 7.32 (t br, 1 H); 4.95 (s br, 1 H); 3.18-3.34 (m, 4 H); 1.53 (s br, 4 H).

MS (ESI): [M+H]⁺=541; Cl isotopes well displayed

### Intermediate 17

### Preparation of (RS)—S-(3-amino-5-nitrophenyl)-S-{4-[(5-iodo-2-chloropyrimidin-4-yl)amino]butyl}sulfoximide

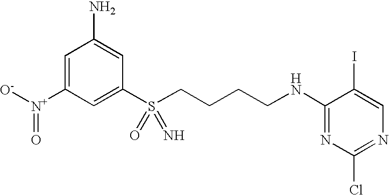

To a solution of (RS)—S-(3,5-dinitrophenyl)-S-{4-[(5-iodo-2-chloropyrimidin-4-yl)amino]butyl}sulfoximide (500 mg, 0.92 mmol) in THF (42 mL) was added a 15% solution of titanium (III) chloride in 10% aq hydrochloric acid (4.62 mL, 5.9 eq) at 0° C. over 30 minutes. The mixture was stirred for 2 h whilst being allowed to warm up to room temperature. The mixture was cautiously poured into a mixture of aq 1 N sodium hydroxide (50 mL) and ice (25 g), followed by extraction with ethyl acetate (3×50 mL). The combined organic layers were washed with brine (50 mL), dried over magnesium sulphate, and evaporated. Trituration of the crude residue with methanol (9 mL) gave the desired aniline (393 mg, 0.77 mmol, 83% yield).

HNMR (DMSO, 300 MHz): 8.23 (s, 1 H); 7.59-7.63 (m, 1 H); 7.53 (t, 1 H); 7.37 -7.41 (m, 1 H); 7.30 (t br, 1 H); 6.25 (s br, 2 H); 4.32 (s, 1 H); 3.22-3.30 (m, 2 H); 3.09-3.18 (m, 2 H); 1.43-1.61 (m, 4 H).

MS (ESI): [M+H]⁺=511; Cl isotopes well displayed

### Intermediate 18

### Preparation of (RS)—S-(3,5-dinitrophenyl)-S-{4-[(5-lodo-2-chloropyrimidin-4-yl)amino]butyl}-N-(benzyloxycarbonyl)-sulfoximide

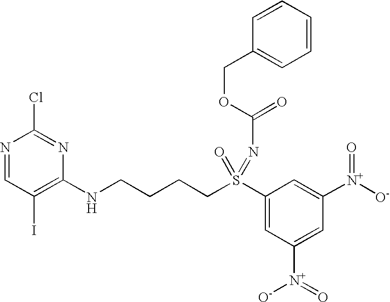

A solution of (RS)—S-(3,5-dinitrophenyl)-S-{4-[(5-iodo-2-chloropyrimidin-4-yl)amino]butyl}-sulfoximide (500 mg, 0.92 mmol; intermediate 16) in pyridine (9.25 mL) was treated dropwise with a solution of benzyl chloroformiate (330 μL, 2.31 mmol) in DCM (0.9 mL) at 0° C. The resulting mixture was stirred for 1 h at room temperature and was then evaporated, followed addition of water (10 mL) and extraction with ethyl acetate (2×25 mL). The combined organic layers were washed with brine and were then dried over sodium sulphate and concentrated. The residue was triturated with hexane containing a trace of ethyl acetate, followed by recrystallisation from acetonitrile to give the pure product (390 mg, 0.58 mmol, 63% yield).

¹H-NMR (DMSO, 300 MHz): 9.03 (t, 1 H); 8.87 (d, 2 H); 8.24 (s, 1 H); 7.33 (t br, 1 H); 7.20-7.28 (m, 4 H); 7.10-7.19 (m, 1 H); 4.88 (mc, 2 H); 3.77-4.04 (m, 2 H); 3.30-3.40 (m, 2 H); covered by water peak); 1.44-1.75 (m, 4 H);

MS (ESI): [M+H]⁺=675 (Cl isotopes well displayed).

### Intermediate 19

### Preparation of (RS)—S-{4-[(5-lodo-2-chloropyrimidin-4-yl)amino]butyl}-N-(benzyloxycarbonyl)-S-(3-nitrophenyl)sulfoximide

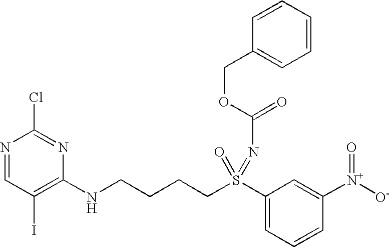

A solution of (RS)—S-{4-[(5-iodo-2-chloropyrimidin-4-yl)amino]butyl}-S-(3-nitrophenyl)sulfoximide (1.87 g, 3.77 mmol; intermediate 5) in pyridine (37 mL) was treated dropwise with a solution of benzyl chloroformiate (1.35 mL, 9.43 mmol) in DCM (3.8 mL) at 0° C. The resulting mixture was stirred for 1.5 h at room temperature and was then poured into water (25 mL), followed by extraction with ethyl acetate (2×100 mL). The combined organic layers were dried over magnesium sulphate and concentrated. The residue was purified by column chromatography to give the desired product (2.38 g, 3.77 mmol, quant. yield).

¹H-NMR (DMSO, 300 MHz): 8.26 (s, 1 H); 7.12-7.38 (m, 7 H); 7.02 (t, 1 H)M 6.89 (d, 1 H); 6.80 (dd, 1 H); 5.68 (s br, 2 H); 4.92 (mc, 2 H); 3.37-3.60 (m, 2 H); 3.19-3.31 (m, 2 H); 1.39-1.62 (m, 4 H).

MS (ESI): [M+H]⁺=630.

### Intermediate 20

### Preparation of (RS)—S-(3-Aminophenyl)-S-{4-[(5-iodo-2-chloropyrimidin-4-yl)amino]butyl}-N-(benzyloxycarbonyl)sulfoximide

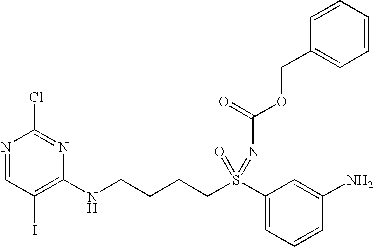

To a solution of (RS)—S-{4-[(5-iodo-2-chloropyrimidin-4-yl)amino]butyl}-N-(benzyloxycarbonyl)-S-(3-nitrophenyl)sulfoximide (810 mg, 1.29 mmol) was added a 15% solution of titanium (III) chloride in approx. 10% aqueous hydrochloric acid (4.4 mL, 5.1 mmol, Merck) at room temperature under an atmosphere of nitrogen. Two further portions of titanium (III) solution (1.1 mL, 1.3 mmol each) were added after 30 min and 45 min stirring at room temperature, respectively. Stirring was then continued for 30 min before the reaction mixture was carefully poured into 2 N aqueous sodium hydroxide (50 mL), followed by extraction with ethyl acetate (3×100 mL). The combined organic layers were dried over sodium sulphate and concentrated in vacuo. Column chromatography gave the desired amine (464 mg, 0.77 mmol, 60% yield).

¹H-NMR (CDCl₃, 300 MHz): 8.72 (t, 1 H); 8.44-8.53 (m, 1 H); 8.20-8.31 (m, 2 H); 7.78 (t, 1 H); 7.21-7.36 (m, 6 H); 5.53 (t br, 1 H); 5.11 (d, 1 H); 4.98 (d, 1 H); 3.33-3.62 (m, 4 H); 1.62-1.98 (m, 4 H).

MS (ESI): [M+H]⁺=600.

### Intermediate 21

### Preparation of Benzyl (RS)—N-[1⁵-iodo-4-oxo-4λ⁶-thia-2,9-diaza-1(2,4)-pyrimidina-3(1,3)-benzenacyclononaphan-4-ylidene]carbamate

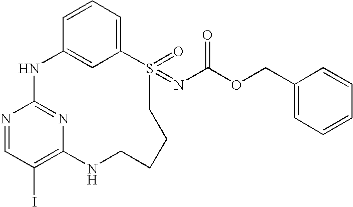

A solution of (RS)—S-(3-aminophenyl)-S-{4-[(5-iodo-2-chloropyrimidin-4-yl)amino]butyl}-N-(benzyloxycarbonyl)sulfoximide (450 mg, 0.75 mmol) in acetonitrile (12 mL) and water (1.2 mL) was added to a mixture of acetonitrile/water/4 M solution of HCl in 1,4-dioxane (107 mL/12 mL/1.2 mL) at a temperature of 50° C. over a period of 4 h by means of a syringe pump. The mixture was then stirred at room temperature overnight and subsequently concentrated in vacuo to a volume of approx. 10 mL. Aqueous sodium carbonate (20 mL) was added and the precipitate was isolated by filtration. The product was washed with water (3 mL) and dried in vacuo (327 mg, 0.58 mmol, 77% yield).

¹H-NMR (DMSO, 400 MHz): 9.60 (s, 1 H); 8.32 (s, 1 H); 8.12 (s, 1 H); 7.52 (t, 1 H); 7.40-7.46 (m, 1 H); 7.17-7.35 (m, 6 H); 6.94 (t br, 1 H); 4.95 (s, 2 H); 3.62-3.83 (m, 2 H); 2.90-3.11 (m, 2 H); 1.49-1.80 (m, 4 H).

MS (ESI): [M+H]⁺=564.

### Intermediate 22

### Preparation of 1-(2,4-Dimethylphenyl)-2-[4-(4,4,5,5-tetramethyl-1,3,2-dioxaborolan-2-yl)phenoxy]ethan-1-one

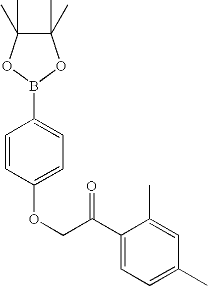

A solution of 4-(4,4,5,5-tetramethyl-1,3,2-dioxaboran-2-yl)phenol (4.40 g, 20 mmol) in DMF (47 mL) was treated with potassium carbonate (3.32 g, 24 mmol), followed by 2-bromo-1-(2,4-dimethylphenyl)ethan-1-one (5.00 g, 22 mmol) under an atmosphere of nitrogen. The resulting mixture was stirred overnight at room temperature and was then evaporated. The residue was partitioned between ethyl acetate and water, and the organic layer was dried and concentrated. The crude residue was subjected to column chromatography to give the pure product (5.87 g, 16.0 mmol, 80% yield).

¹H-NMR (CDCl₃, 300 MHz): 7.81 (d, 2 H); 7.55 (d, 2 H); 7.13 (d, 2 H); 6.88 (d, 2 H); 5.39 (s, 2 H); 2.35 (s, 3 H); 2.29 (s, 3 H); 1.23 (s, 12 H).

### Intermediate 23

### Preparation of Benzyl N—[(RS)-1⁵-{4-[(2,4-dimethylbenzoyl)methoxy]-phenyl}-4-oxo-4λ⁶-thia-2,9-diaza-1(2,4)-pyrimidina-3(1,3)-benzenacyclononaphan-4-ylidene]carbamate

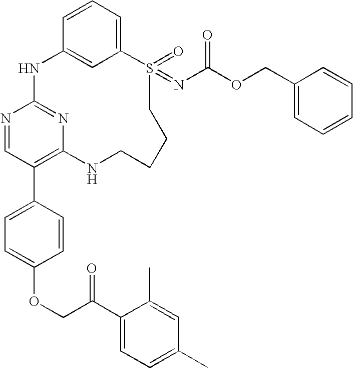

To a solution of benzyl (RS)—N-[1⁵-iodo-4-oxo-4X⁶-thia-2,9-diaza-1(2,4)-pyrimidina-3(1,3)-benzenacyclononaphan-4-ylidene]carbamate (141 mg, 0.25 mmol) in DMF (4 mL) was added 1-(2,4-dimethylphenyl)-2-[4-(4,4,5,5-tetramethyl-1,3,2-dioxaborolan-2-yl)phenoxy]ethan-1-one (114 mg, 0.31 mmol), followed by a solution of potassium carbonate (86 mg, 0.62 mmol) in water (0.33 mL), and POPd (31 mg, 0.062 mmol) under an atmosphere of nitrogen. The mixture was stirred at 100° C. for 4 h; after cooling to room temperature, the mixture was evaporated and triturated with water (7 mL). The residue was purified by HPLC to give the target compound (17 mg, 0.025 mmol, 10% yield).

¹H-NMR (CDCl₃, 300 MHz): 8.48 (m, 1 H); 7.61-7.72 (m, 3 H); 7.52 (t, 1 H); 7.19-7.38 (m, 10 H); 7.08-7.16 (m, 2 H); 7.00 (d, 2 H); 5.63 (t br, 1 H); 5.24 (s, 2 H); 5.11 (mc, 2 H); 3.58-3.75 (m, 2 H); 2.97-3.22 (m, 2 H); 2.53 (s, 3 H); 2.38 (s, 3 H); 1.55-2.08 (m, 4 H).

### Intermediate 24

### Preparation of 2-[4-(3,5-Dinitro-phenylsulfanyl)-butyl]-isoindole-1,3-dione

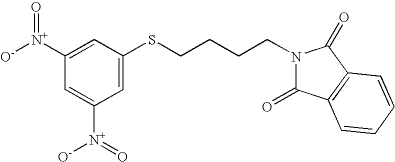

To a solution of 3,5-dinitrothiophenol (20.0 g; 100 mmol) in ethanol (750 mL) was added sodium hydroxide (4.8 g; 120 mmol), and the mixture was stirred at room temperature for 5 min. Subsequently, N-(4-bromobutyl)phthalimide (31.0 g; 110 mmol) was added portionwise over 5 min, followed by stirring at room temperature for 21 h, during which a precipitate formed. The precipitate was isolated by filtration, triturated with water (600 mL), and dried to give 30.3 g of target compound; an additional crop (3.2 g) was isolated from the evaporated mother Liquor by column chromatography. Total yield 33.5 g (82.9 mmol, 83%).

¹H-NMR (DMSO, 300 MHz): 8.47 (t, 1 H); 8.38 (d, 2 H); 7.73-7.88 (m, 4 H); 3.58 (t, 2 H); 3.24 (t, 3 H); 1.54-1.84 (m, 4 H).

### Intermediate 25

### Preparation of 2-[4-(3-Amino-5-nitro-phenylsulfanyl)-butyl]-isoindole-1,3-dione

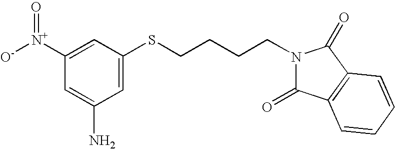

To a solution of 2-[4-(3,5-dinitro-phenylsulfanyl)-butyl]-isoindole-1,3-dione (10.0 g; 25 mmol) in THF (1100 mL) was added a 10% solution of titanium (III) chloride in approx. 20% aqueous hydrochloric acid (259 mL, 200 mmol, Aldrich) over a period of 60 min whilst maintaining the temperature of the mixture at 10° C. Stirring at 10° C. was continued for 2 h, after which another 26 mL (20 mmol) of the titanium (III) chloride solution was added. After stirring at 10° C. for another hour, the reaction mixture was very carefully and portionwise poured into 4 N aqueous sodium hydroxide (800 mL), followed by stirring at room temperature for 15 min. The mixture was then carefully extracted with ethyl acetate (4×500 mL); the combined organic layers were washed with brine and concentrated in vacuo. The crude product was purified by column chromatography, followed by trituration with diethyl ether to give the pure target compound (3.76 g, 10.1 mmol, 40% yield.

¹H-NMR (DMSO, 400 MHz): 7.80 (mc, 4 H); 7.04-7.12 (m, 2 H); 6.68-6.72 (m, 1 H); 5.84 (s br, 2 H); 3.55 (t, 2 H); 2.97 (t, 2 H); 1.63-1.76 (m, 2 H); 1.49-1.61 (m, 2 H).

MS (ESI): [M+H]⁺=372.

### Intermediate 26

### Preparation of Pyrrolidine-1-carboxylic acid {3-[4-(1,3-dioxo-1,3-dihydro-isoindol-2-yl)-butylsulfanyl]-5-nitro-phenyl}-amide

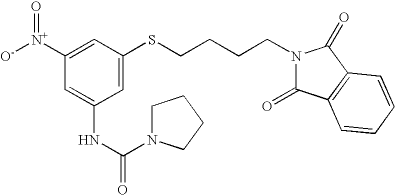

To a solution of 2-[4-(3-amino-5-nitro-phenylsulfanyl)-butyl]-isoindole-1,3-dione (7.43 g; 20 mmol) in DCM (660 mL) was added TEA (2.77 mL; 20 mmol), followed by triphosgene (2.97 g; 10 mmol) at a temperature of 5° C. The resulting mixture was stirred 5 min at 5° C., after which the cooling bath was removed and the mixture was stirred for another 1.5 h at room temperature. Another portion of TEA (0.47 mL; 3.4 mmol) and triphosgene (1.00 g; 3.4 mmol) was added and stirring at room temperature was continued for 1 h, followed by evaporation. The residue was treated with DCM (660 mL), followed by pyrrolidine (2.48 mL, 30 mmol), and the mixture was stirred overnight. After evaporation, the crude product was purified by column chromatography to give the desired urea (8.52 g, 18.2 mmol, 91% yield).

¹H-NMR (DMSO, 400 MHz): 8.55 (s, 1 H); 8.24-8.30 (m, 1 H); 7.84-7.90 (m, 1 H); 7.80 (mc, 4 H); 7.49-7.55 (m, 1 H); 3.56 (t, 2 H); 3.28-3.40 (m, 4 H); 3.02 (t, 2 H); 1.51-1.92 (m, 8 H).

MS (ESI): [M+H]⁺=469.

### Intermediate 27

### Preparation of (RS)-Pyrrolidine-1-carboxylic acid {3-[4-(1,3-dioxo-1,3-dihydro-isoindol-2-yl)-butylsulfinyl]-5-nitro-phenyl}-amide

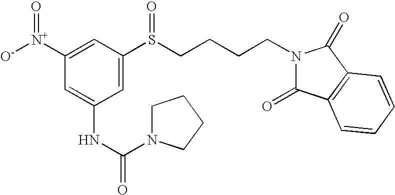

To a solution of pyrrolidine-1-carboxylic acid {3-[4-(1,3-dioxo-1,3-dihydro-isoindol-2-yl)-butylsulfanyl]-5-nitro-phenyl}-amide (7.97 g; 17 mmol) in acetonitrile (170 mL) was added anhydrous ferric chloride (83 mg; 0.51 mmol), and periodic acid (4.11 g; 18.0 mmol), and the mixture was stirred at room temperature for 2 h. The reaction was stopped by the addition of a solution of sodium thiosulphate pentahydrate (17 g; 108 mmol) in water (120 mL), after which stirring at room temperature was continued for another 30 min. Acetonitrile was removed in vacuo, and the remaining aqueous mixture was extracted with DCM (4×150 mL), and the combined organic layers were washed with brine, dried and evaporated. The crude sulfoxide was purified by column chromatography to give the pure target compound (6.87 g, 14.2 mmol, 83% yield).

¹H-NMR (DMSO, 400 MHz): 8.89 (s, 1 H); 8.61-8.67 (m, 1 H); 8.16-8.22 (m, 1 H); 7.90-7.95 (m, 1 H); 7.79 (s, 4 H); 3.53 (t, 2 H); 3.31-3.44 (m, 4 H); 3.01-3.12 (m, 1 H); 2.74-2.84 (m, 1 H); 1.77-1.90 (m, 4 H); 1.55-1.74 (m, 23 H); 1.33-1.48 (m, 1 H).

MS (ESI): [M+H]⁺=485.

### Intermediate 28

### Preparation of N-{3-[(RS)—S-[4-(1,3-Dioxo-2,3-dihydro-1H-isoindol-2-yl)butyl]-N-{[2-(trimethylsilyl)ethyl]sulfonyl}sulfonimidoyl]-5-nitrophenyl}pyrrolidine-1-carboxamide

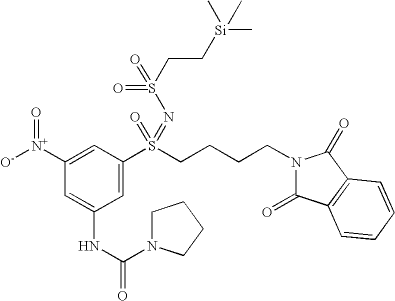

To a solution of (RS)-pyrrolidine-1-carboxylic acid {3-[4-(1,3-dioxo-1,3-dihydro-isoindol-2-yl)-butylsulfinyl]-5-nitro-phenyl)-amide (2.42 g; 5.00 mmol) in acetonitrile (380 mL) was added tetrakis-(acetonitrile)copper-(I)-hexafluorophosphate (93.2 mg; 0.25 mmol) at 0° C. under rigorous exclusion of oxygen and moisture. Subsequently, [N-(2-(Trimethylsilyl)ethanesulfonyl)-phenyl]iodinane (1.92 g; 5.00 mmol; prepared according to P. Dauban and R H Dodd, J. Org. Chem. 1999, 64, 5304) was added under ice cooling and the mixture was stirred at 0° C. for 2.5. h, followed by the addition of another portion of tetrakis-(acetonitrile)copper-(I)-hexafluorophosphate (93.2 mg; 0.25 mmol) and [N-(2-(Trimethylsilyl)ethanesulfonyl)-phenyl]iodinane (1.92 g; 5.0 mmol). After stirring under ice cooling for another 1.5 h; another addition of tetrakis-(acetonitrile)copper-(I)-hexafluorophosphate (46.6 mg; 0.13 mmol) and [N-(2-(Trimethylsilyl)ethanesulfonyl)-phenyl]iodinane (0.96 g; 2.5 mmol) was carried out. The reaction mixture was stirred overnight at 0° C. and was then evaporated carefully. Column chromatography of the crude residue gave the desired sulfoximine (1.81 g, 2.73 mmol, 54% yield).

¹H-NMR (DMSO, 300 MHz): 9.06 (s, 1 H); 8.82-8.87 (m, 1 H); 8.51-8.57 (m, 1 H); 8.12-8.16 (m, 1 H); 7.78 (mc,.4 H); 3.66-3.89 (m, 2 H); 3.44-3.56 (m, 2 H); 3.30-3.42 (m, 4 H); 2.94 (mc, 2 H); 1.79-1.92 (m, 4 H); 1.47-1.71 (m, 4 H); 0.92 (mc, 2 H); −0.03 (s, 9 H).

MS (ESI): [M+H]⁺=664.

### Intermediate 29

### Preparation of N-{3-[(RS)—S-{4-[(2-Chloro-5-iodopyrimidin-4-yl)amino]butyl}-N-{[2-(trimethylsilyl)ethyl]sulfonyl}sulfonimidoyl]-5-nitrophenyl}pyrrolidine-1-carboxamide

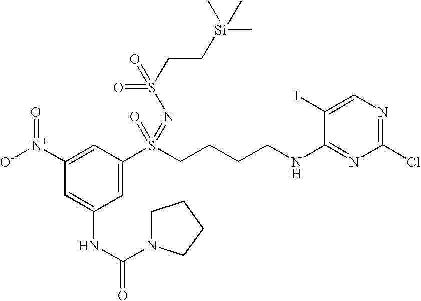

To a solution of N-{3-[(RS)—S-[4-(1,3-dioxo-2,3-dihydro-1H-isoindol-2-yl)butyl]-N-{[2-(trimethylsilyl)ethyl]sulfonyl}sulfonimidoyl]-5-nitrophenyl}pyrrolidine-1-carboxamide (4.12 g; 6.20 mmol) in ethanol (400 mL) was added hydrazine monohydrate (6.2 mL, 128 mmol), and the mixture was stirred at room temperature for 2.5 h. Ethanol was evaporated very carefully, maintaining bath temperature below 35° C., and the residue was dissolved in DCM (150 mL). The solution was washed with water (50 mL) and brine (50 mL), and was then dried and evaporated. The crude residue (4.08 g) was then dissolved in acetonitrile (38 mL), and TEA (1.72 mL, 12.4 mmol), followed by 2,4-dichloro-5-iodopyrimidine (1.96 g; 7.13 mmol). The mixture was stirred for 3 h at room temperature, and then evaporated. The crude residue was purified by column chromatography to give the desired target product (1.54 g, 1.99 mmol, 31% yield).

¹H-NMR (DMSO, 300 MHz): 9.09 (s, 1 H); 8.82-8.86 (m, 1 H); 8.57-8.62 (m, 1 H); 8.23 (s, 1 H); 7.29 (t br, 1 H); 3.68-3.90 (m, 2 H); 3.33-3.42 (m, 4 H); 3.24-3.30 (m, 2 H; covered by water peak); 2.93-3.00 (m, 2 H); 1.79-1.90 (m, 4 H); 1.45-1.65 (m, 2 H); 0.86-0.97 (m, 2 H); −0.02 (s, 9 H).

MS (ESI): [M+H]⁺=772; Cl isotopes well displayed.

### Intermediate 30

### Preparation of N-{3-Amino-5-[(RS)—S-{4-[(2-chloro-5-iodopyrimidin-4-yl)amino]butyl}-N-{[2-(trimethylsilyl)ethyl]sulfonyl}sulfonimidoyl]phenyl}-pyrrolidine-1-carboxamide

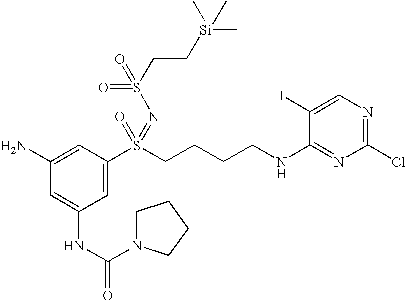

To a solution of N-{3-[(RS)—S-{4-[(2-chloro-5-iodopyrimidin-4-yl)amino]butyl}-N-{[2-(trimethylsilyl)ethyl]sulfonyl}sulfonimidoyl]-5-nitrophenyl}pyrrolidine-1-carboxamide (1.23 g; 1.59 mmol) in THF (75 mL) was added a 10% solution of titanium (III) chloride in aqueous 20-30% hydrochloric acid (16.5 mL; 12.7 mmol) at room temperature over a period of 5 minutes. The mixture was stirred for 1 h at room temperature before it was carefully poured into a mixture of ice (125 g) and aqueous 4 N sodium hydroxide (60 mL). The mixture was stirred for 10 min with ethyl acetate (200 mL) and the layers were separated. The aqueous layer was again treated with 4 N sodium hydroxide (10 mL) to maintain pH 10, followed by repeated extraction with ethyl acetate (4×150 mL). The combined organic layers were washed with brine, dried and evaporated. Column chromatography of the crude residue gave the desired amine (858 mg, 1.16 mmol, 73% yield).

¹H-NMR (DMSO, 400 MHz): 8.26 (s, 1 H); 8.23 (s br, 1 H); 7.33 (t br, 1 H); 7.21 -7.27 (m, 1 H); 7.11-7.17 (m, 1 H); 6.62-6.66 (m, 1 H); 5.53-5.62 (m, 2 H); 3.53-3.63 (m, 1 H); 3.41-3.52 (m, 1 H); 3.21-3.36 (m, 6 H, partly covered by water peak); 2.85-2.96 (m, 2 H); 1.74-1.87 (m, 4 H); 1.45-1.64 (m, 4 H); 0.85-0.94 (M, 2 H); −0.03 (s, 9 H).

MS (ESI): [M+H]⁺=742; Cl isotopes well displayed.

### Intermediate 31

### Preparation of N—[(RS)-1⁵-lodo-4-oxo-4-({[2-(trimethylsilyl)ethyl]sulfonyl}imino)-4λ⁶-thia-2,9-diaza-1(2,4)-pyrimidina-3(1,3)-benzenacyclononaphan-3⁵-yl]pyrrolidine-1-carboxamide

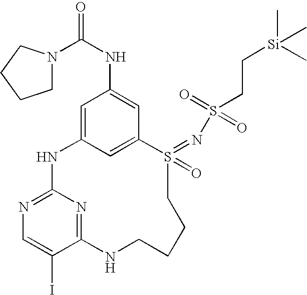

A solution of N-{3-amino-5-[(RS)—S-{4-[(2-chloro-5-iodopyrimidin-4-yl)amino]butyl}-N-{[2-(trimethylsilyl)ethyl]sulfonyl}sulfonimidoyl]phenyl}-pyrrolidine-1-carboxamide (850 mg, 1.15 mmol) in a mixture of 2-butanol (0.78 mL), water (2.62 mL) and acetonitrile (23.5 mL) was added over 30 min to a mixture of water (13.1 mL), acetonitrile (118 mL) and a 4 N solution of hydrogen chloride in 1,4-dioxane (1.63 mL, 6.54 mmol) at room temperature under an inert atmosphere. The mixture was heated at reflux for 3.5 h. After cooling to room temperature, the mixture was evaporated and then triturated with aq. sodium bicarbonate for 15 min. The crude product was isolated by filtration and then purified by column chromatography to give the pure target compound (474 mg, 0.67 mmol, 59% yield).

¹H-NMR (DMSO, 300 MHz): 9.53 (s, 1 H); 8.62 (s, 1 H); 8.14 (s, 1 H); 7.87-7.94 (m, 1 H); 7.77-7.81 (m, 1 H); 7.62-7.68 (m, 1 H); 6.99 (t br, 1 H); 3.72-3.86 (m, 2 H); 3.33-3.45 (m, 4 H); 2.91-3.12 (m, 4 H); 1.82-1.94 (m, 4 H); 1.70-1.81 (m, 2 H); 1.56-1.68 (m, 2 H); 0.95 (mc, 2 H); 0.01 (s, 9 H).

MS (ESI): [M+H]⁺=706.

### Intermediate 32

### Preparation of N—[(RS)-1⁵-(4-Ethoxyphenyl)-4-oxo-4-({[2-(trimethylsilyl)ethyl]sulfonyl}imino)-4λ⁶-thia-2,9-diaza-1(2,4)-pyrimidina-3(1,3)-benzenacyclononaphan-3⁵-yl]pyrrolidine-1-carboxamide

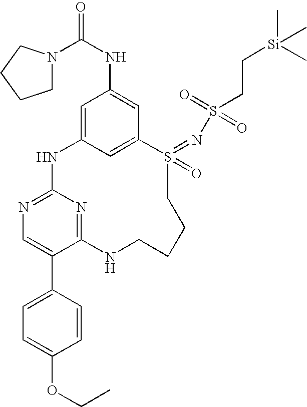

To a mixture of N—[(RS)-1⁵-iodo-4-oxo-4-({[2-(trimethylsilyl)ethyl]sulfonyl}imino)-4λ⁶-thia-2,9-diaza-1(2,4)-pyrimidina-3(1,3)-benzenacyclononaphan-3⁵-yl]pyrrolidine-1-carboxamide (32 mg, 45 μmol), 4-ethoxyphenyl boronic acid (19 mg, 0.11 mmol) and POPd (4.1 mg, 8.2 μmol) in DMF (0.9 mL) was added a solution of potassium carbonate (15 mg, 0.11 mmol) in water (56 μL). The mixture was stirred at 100° C. for 2 h. After cooling to room temperature, the mixture was evaporated and the crude residue was triturated with water for 30 min. The crude product was isolated by filtration, followed by purification by column chromatography to give the pure target compound (15 mg, 21 μmol, 48% yield).

¹H-NMR (DMSO, 300 MHz): 9.51 (s, 1 H); 8.56 (s, 1 H);7.97-8.03 (m, 1 H); 7.67-7.75 (m, 2 H); 7.58-7.64 (m, 1 H); 7.24 (d, 2 H); 6.97 (d, 2 H); 4.02 (q, 2 H); 3.71-3.84 (m, 2 H); 3.31-3.42 (m, 4 H); 2.87-3.04 (m, 4 H); 1.70-1.91 (m, 6 H); 1.54-1.67 (m, 2 H); 1.31 (t, 3 H); 0.92 (mc, 2 H); −0.05 (s, 9 H).

MS (ESI): [M+H]⁺=700.

### Intermediate 33

### Preparation of 1-Phenyl-N-[4-(4,4,5,5-tetramethyl-1,3,2-dioxaborolan-2-yl)phenyl]cyclo-propanecarboxamide

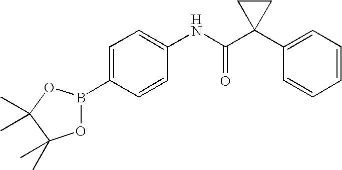

To a solution of 4-(4,4,5,5-tetramethyl-1,3,2-dioxoborolan-2-yl)-aniline (636 mg, 2.9 mmol) in pyridine (8.9 mL) was added 1-phenylcyclopropane carbonyl chloride( 1.5 eq, 4.36 mmol; prepared form the respective carboxylic acid by reaction with 6 eq thionyl chloride, followed by evaporation) in pyridine (4.36 mL). The resulting mixture was stirred at room temperature for 2 days. The volatiles were removed in vacuo, the residue was purified by column chromatography on silica gel to give 706 mg (1.94 mmol, 67%) of the desired amide.

¹H-NMR (DMSO, 300 MHz): 9.09 (s, 1 H); 7.52 (br. s 4 H); 7.22-7.38 (m, 5 H); 1.39-1.43 (m, 2 H); 1.23 (s, 12 H); 1.06-1.10 (m, 2 H).

MS (ESI): [M+H]⁺=364.

### Intermediate 34

### Preparation of N-butynyl-2,3-dichlorobenzene sulfonamide

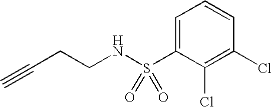

To a suspension of 3-butynylamine hydrochloride (422 mg, 4.00 mmol) in acetonitrile (12 mL) was added TEA (0.83 mL, 6.00 mmol), followed by portionwise addition of 2,3-dichlorobenzene sulfonyl chloride (982 mg, 4.00 mmol) over a period of 30 min at room temperature. The mixture was stirred at room temperature for 6 h, after which another 0.28 mL of TEA was added, and further stirred overnight. Water was added, and the mixture was extracted with ethyl acetate (4×). The combined organic layers were washed with water and brine, filtered, and dried to give the crude sulphonamide (1.11 g, 4.00 mmol, quant. yield) which was used without further purification.

¹H-NMR (DMSO, 300 MHz): 8.23 (s, br, 1 H); 7.86-7.96 (m, 2 H); 7.52 (t, 1 H); 2.98 (t, 2 H); 2.73 (t, 1 H); 2.24 (dt, 2 H).

### Intermediate 35

### Preparation of 2,3-Dichloro-N-[4-(4,4,5,5-tetramethyl-1,3,2-dioxaborolan-2-yl)phenyl]benzenesulfonamide

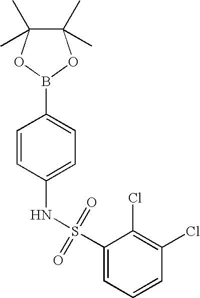

To a solution of 4-(4,4,5,5,tetramethyl-[1,3,2]dioxaborolan-2-yl)-aniline (1.31 g; 6.00 mmol) in DCM (30 mL) was added pyridine (0.72 mL, 9.00 mmol), followed by 2,3-dichlorobenzene sulfonyl chloride (1.55 g; 6.30 mmol). The reaction mixture was stirred overnight at room temperature and was then concentrated in vacuo. The crude residue was purified by column chromatography to give the desired sulphonamide (2.37 g, 5.54 mmol, 92% yield).

hu 1H-NMR (CDCl₃, 300 MHz): 7.87-7.93 (m, 2 H); 7.61 (d, 2 H); 7.51-7.57 (m, 2 H); 7.29 (t, 1 H); 6.98-7.07 (m, 3 H); 1.24 (s, 12 H).

MS (ESI): [M+H]⁺=428 (Cl isotopes well displayed).

Intermediates 36 to 40 were prepared according to general procedure GP1 from 4-(4,4,5,5-tetramethyl-1,3,2-dioxaborolyan-2-yl)-aniline and the respective isocyanate.

| Inter- | | | |
|---|---|---|---|
| mediate | | | |
| No | Structure And Yield | Name | Analytical Data |
| 36 | 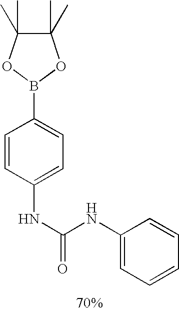 | N-phenyl-N′-[4- (4,4,5,5- tetramethyl-1,3,2- dioxaborolan-2- yl)phenyl]urea | |
| #¹H-NMR (CDCl₃): 7.72(d, 2H); 7.21-7.33(m, 6H); 6.98-7.06(m, 3H); 1.34 (s, 12H). MS (ESI): [M + H]⁺ = 339. | | | |
| 37 | 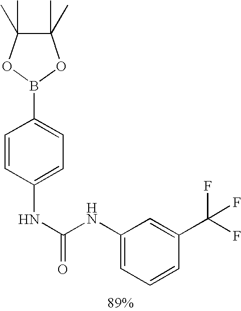 | N-[4-(4,4,5,5- tetramethyl-1,3,2- dioxaborolan-2- yl)phenyl]-N′-[3- (trifluoromethyl) phenyl]urea | |
| #¹H-NMR (CDCl₃, 300 MHz): 7.74(d, 2H); 7.58-7.68(m, 1H): 7.16-7.55(m, 7H); 1.32(s, 12H). MS (ESI): [M + H]⁺ = 407. | | | |
| 38 | 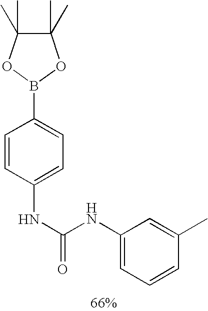 | N-(3- methylphenyl)-N′- [4-(4,4,5,5- tetramethyl-1,3,2- dioxaborolan-2- yl)phenyl]urea | |
| #¹H-NMR (DMSO): 8.74(s, 1H); 8.57(s, 1H); 7.55 (d, 2H); 7.44(d, 2H); 7.26(s, 1H); 7.06-7.22 (m, 2H); 6.77(d, 1H); 2.25(s, 3H); 1.27(s, 12H). MS (ESI): [M + H]⁺ = 353. | | | |
| 39 | 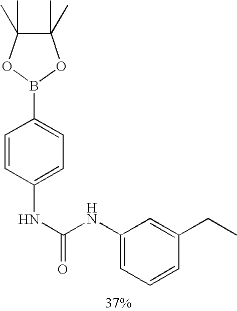 | N-(3-ethylphenyl)- N′-[4-(4,4,5,5- tetramethyl-1,3,2- dioxaborolan-2- yl)phenyl]urea | |
| #¹H-NMR (CDCl₃): 7.73(d, 2H); 7.33(d, 2H); 7.20 (t, 1H); 7.14(s br, 1H); 7.06-7.13(m, 2H); 6.99 (s br, 1H); 6.93(d, 1H); 2.58(q, 2H); 1.32(s, 12H); 1.18(t, 3H). MS (ESI): [M + H]⁺ = 367. | | | |
| 40 | 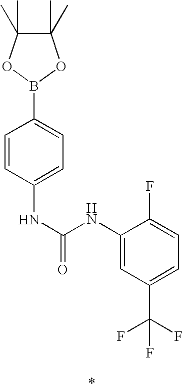 | N-[2-fluoro-5- (trifluoromethyl) phenyl]-N′-[4- (4,4,5,5- tetramethyl-1,3,2- dioxaborolan-2- yl)phenyl]urea | |

*used crude without further characterisation

### PREPARATION OF EXAMPLE COMPOUNDS

### Example 1.1

### Preparation of (RS)-1⁵-Bromo-4-imino-4-thia-2,9-diaza-1(2,4)-pyrimidina-3(1,3)-benzenacyclononaphane 4-oxide

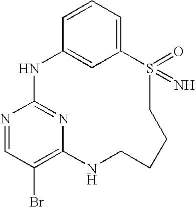

Ethyl (RS)—N-[1⁵-bromo-4-oxo-4λ⁶-thia-2,9-diaza-1(2,4)-pyrimidina-3(1,3)-benzenacyclononaphan-4-ylidene]carbamate (81 mg; 0.18 mmol) was treated with a 0.125 M solution of sodium ethoxide in ethanol (4 mL) and heated to 70° C. After 3 h and 4 h, portions of the 0.125 M solution of sodium ethoxide in ethanol (1 mL each) were added. Stirring at 70° C. was continued for another 5 h prior to cooling to room temperature, dilution with ethyl acetate, and the addition of brine. The resulting mixture was extracted with ethyl acetate (3×). The combined organic layers were dried (Na₂SO₄), filtered and evaporated. The residue was purified by column chromatography (DCM/EtOH 9:1) to give 35 mg (0.09 mmol, 52% yield) of the title compound.

¹H-NMR (DMSO): 9.52 (s, 1H), 8.39 (m, 1H), 8.03 (s, 1H), 7.46 (m, 2H), 7.28 (m, 2H), 4.20 (s, 1H), 3.30 (m, 2H), 3.04 (m, 2H), 1.70 (m, 4H).

MS (ESI):[M+H]⁺=382.

The racemic title compound was separated into its enantiomers by preparative HPLC:

| | |
|---|---|
| Column: | Chiralcel OJ 20μ |
| Length × internal diameter: | 290 × 50.8 mm |
| Eluents: | Hexane/Ethanol 70:30 |
| Flow rate: | 80.0 mL/min |
| Detector: | UV 280 nm |
| Temperature: | room temperature |
| Retention time [min]: | 43.7: Enantiomer 1 (Expl. 1a) |
| | 55.9: Enantiomer 2 (Expl. 1b) |

### Example 1.2

### Preparation of (RS)-1⁵-lodo-4-imino-4-thia-2,9-diaza-1(2,4)-pyrimidina-3(1,3)-benzenacyclononaphane 4-oxide

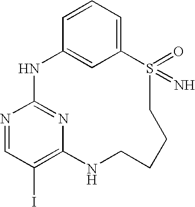

A solution of (RS)—S-(3-aminophenyl)-S-{4-[(5-iodo-2-chloropyrimidin-4-yl)amino]butyl}sulfoximide (295 mg, 0.63 mmol) in acetonitrile/water (10 mL/1 mL) was added to a mixture of acetonitrile/water/4 M HCl in 1,4-dioxane (90 mL/10 mL/1 mL) at a temperature of 50° C. by means of a syringe pump over a period of 3 h. After 24 h, all volatiles were removed in vacuo and sat. aq. NaHCO₃ solution (50 mL) and ethyl acetate (250 mL) were added. The organic layer was separated and the aqueous layer was extracted with ethyl acetate (3×). The combined organic layers were washed with brine, dried (Na₂SO₄), filtered and evaporated. The crude residue was purified by column chromatography (DCM/EtOH 9:1) to give the title compound (135 mg, 0.32 mmol, 50% yield).

¹H-NMR (DMSO): 9.51 (s, 1H), 8.39 (m, 1H), 8.12 (s, 1H), 7.45 (m, 2H), 7.23 (m, 1H), 6.95 (t, 1H), 4.19 (s, 1H), 3.30 (m, 2H), 3.04 (m, 2H), 1.69 (m, 4H).

MS (ESI):[M+H]⁺=430.

### Example 1.3

### Preparation of (RS)-4-Imino-1⁵-(4-methoxyphenyl)-4-thia-2,9-diaza-1(2,4)-pyrimidina-3(1,3)-benzenacyclononaphane 4-oxide

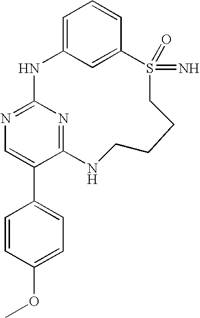

Example 1.3 was prepared according to GP 2 from (RS)-1⁵-lodo-4-imino-4-thia-2,9-diaza-1(2,4)-pyrimidina-3(1,3)-benzenacyclononaphane 4-oxide and 4-methoxyphenylboronic acid. Yield 40%.

¹H-NMR (DMSO): 9.40 (s, 1 H); 8.51 (s, 1 H); 7.70 (s, 1 H); 7.34-7.46 (m, 2 H); 7.17-7.30 (m, 3 H); 6.98 (d, 2 H); 6.68 (t, 1 H); 4.14 (s, 1 H); 3.76 (s, 3 H); 3.27 (mc, 2 H); 2.92-3.06 (m, 2 H); 1.46-1.91 (m, 4 H).

MS (ESI): [M+H]⁺=410.

The racemic title compound was separated into its enantiomers by preparative HPLC:

| | |
|---|---|
| Column: | Chiralcel OJ-H 5μ |
| Length × internal diameter: | 250 × 20 mm |
| Eluents: | Ethanol isocratic |
| Flow rate: | 7.0 mL/min |
| Detector: | UV 214 nm |
| Temperature: | room temperature |
| Retention time [min]: | 18.1: Enantiomer 1 (Expl. 8a) |
| | 36.9: Enantiomer 2 (Expl. 8b) |

The following example compounds were prepared by Suzuki couplings according to according to the general procedure GP 2 from (RS)-1⁵-lodo-4-imino-4-thia-2,9-diaza-1(2,4)-pyrimidina-3(1,3)-benzenacyclononaphane 4-oxide and the respective boronic acid pinacolate esters/free boronic acids. These were either commercially available or prepared as described (intermediates 35 to 40).

| Example | | | |
|---|---|---|---|
| No | Structure And Yield | Name | Analytical Data |
| 1.4 | 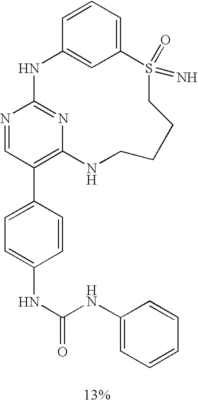 | N-{4-[(RS)-4-Imino- 4-oxo-4λ⁶-thia-2,9- diaza-1(2,4)- pyrimidina-3(1,3)- benzenacyclononaphan- 1⁵-yl]phenyl}- N′-phenyl urea | |
| #¹H-NMR (DMSO): 9.39(s, 1H); 8.73(s, 1H); 8.62(s, 1H); 8.51(s, 1H); 7.73(s, 1H); 7.35-7.58(m, 6H); 7.17-7.32(m, 5H); 6.93 (t, 1H); 4.12(s, 1H); 3.20-3.31(m, 2H); 2.92-3.10 (m, 2H); 1.43-1.97(m, 4H). MS (ESI): [M + H]⁺ = 514. mp. 267° C. (dec.) | | | |
| 1.5 | 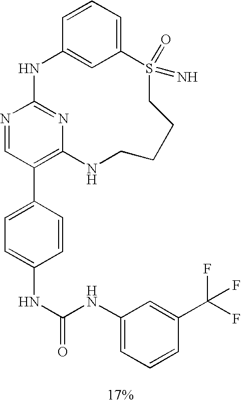 | N-{4-[(RS)-4-Imino- 4-oxo-4λ⁶-thia-2,9- diaza-1(2,4)- pyrimidina-3(1,3)- benzenacyclononaphan- 1⁵-yl]phenyl}- N′-[3-(trifluormethyl) phenyl]urea | |
| #¹H-NMR (DMSO): 9.39(s, 1H); 9.02(s, 1H); 8.87(s, 1H); 8.52(s, 1H); 7.73(s, 1H); 7.36-7.60(m, 6H); 7.21-7.32(m, 4H); 6.74(t br, 1H); 4.12(s, 1H); 3.21 3.32(m, 2H); 2.93-3.08 (m, 2H); 1.43-1.98(m, 4H). MS (ESI): [m + H]⁺ = 582. mp. 253° C. (dec.) | | | |
| 1.6 | 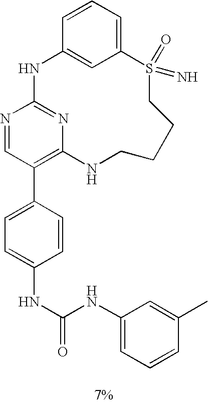 | N-{4-[(RS)-4-Imino- 4-oxo-4λ⁶-thia-2,9- diaza-1(2,4)- pyrimidina-3(1,3)- benzenacyclononaphan- 1⁵-yl]phenyl}- N′-(3-methylphenyl)urea | |
| #¹H-NMR (DMSO): 9.42(s, 1H); 8.73(s, 1H); 8.58(s, 1H); 8.50(s, 1H); 7.73(s, 1H); 7.46-7.55(m, 2H); 7.33-7.44(m, 2H); 7.17-7.32(m, 5H); 7.12(t, 1H); 6.70-6.80(m, 2H); 4.16 (s, 1H); 3.20-3.29(m, 2H); 2.93-3.06(m, 2H); 2.23(s, 3H); 1.68-1.93 (m, 2H); 1.46-1.67(m, 2H). | | | |
| #MS (ESI): [M + H]⁺ = 528. mp. 250° C. (dec.) | | | |
| 1.7 | 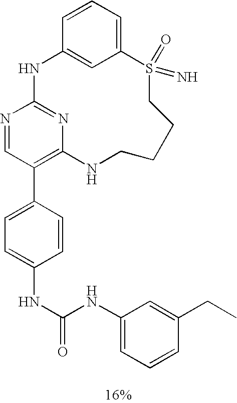 | N-(3-Ethylphenyl)- N′-{4-[(RS)-4-imino- 4-oxo-4λ⁶-thia-2,9- diaza-1(2,4)- pyrimidina-3(1,3)- benzenacyclononaphan- 1⁵-yl]phenyl}urea | |
| #¹H-NMR (DMSO): 9.42(s, 1H); 8.72(s, 1H); 8.59(s, 1H); 8.50(s, 1H); 7.74(s, 1H); 7.48-7.56(m, 2H); 7.33-7.45(m, 2H); 7.30 (s, 1H); 7.19-7.27(m, 4H); 7.15(t, 1H); 6.72-6.82(m, 2H); 4.16(s, 1H); 3.20-3.28(m, 2H); 2.92-3.08(m, 2H); 2.56(q, 2H); 1.70-1.94(m, 2H); 1.44- | | | |
| #1.69(m, 2H); 1.13(t, 3H). MS (ESI): [M + H]⁺ = 542. mp. 225° C. (dec.) | | | |
| 1.8 | 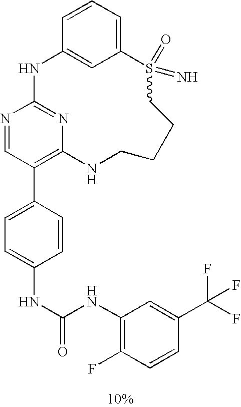 | (RS)-1-[2-Fluoro-5- (trifluoromethyl)phenyl]- 3-{4-[4-imino- 4-oxo-4λ⁶-thia-2,9- diaza-1(2,4)- pyrimidina-3(1,3)- benzenacyclononaphan- 1⁵-yl]phenyl}urea | |
| #¹H-NMR (DMSO): 9.43(s, 1H); 9.25(s, 1H); 8.88(s br, 1H); .8.61(d br, 1H); 8.51(s, 1H); 7.73(s, 1H); 7.19-7.55(m, 9H); 6.77 (t br, 1H); 4.14(s, 1H); 3.20-3.29(m, 2H); 2.92-3.05(m, 2H); 1.45-1.94 (m, 4H). | | | |
| 1.9 | 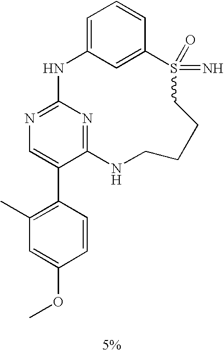 | (RS)-4-Imino-1⁵-(2- methyl-4- methoxyphenyl)-4- thia-2,9-diaza- 1(2,4)-pyrimidina- 3(1,3)- benzenacyclononaphane 4-oxide | |
| #¹H-NMR (DMSO): 9.37(s, 1H); 8.52(s, 1H); 7.56(s, 1H); 7.32-7.45(m, 2H); 7.22(d, 1H); 7.01(d, 1H); 6.85(d, 1H); 6.77(dd, 1H): 6.78(t br, 1H); 4.14 (s, 1H); 3.73(s, 3H); 3.18-3.35(m, 2H); 2.87-3.08 (m, 2H); 2.06(m, 3H); 1.48-1.92(m, 4H). MS (ESI): [M + H]⁺ = 424. | | | |
| 1.10 | 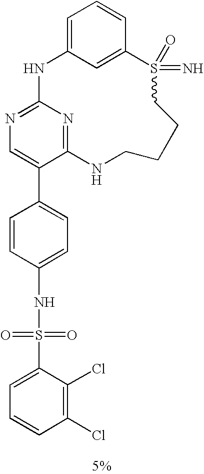 | (RS)-2,3-Dichloro-N- {4-[4-imino-4-oxo-4- thia-2,9-diaza- 1(2,4)-pyrimidina- 3(1,3)- benzenacyclononaphan- 1⁵-yl]phenyl}benzenesulfonamide | |
| #¹H-NMR (DMSO): 11.10(s, 1H); 10.37(s br, 1H); 8.23 (s, 1H); 8.12(d, 1H); 7.94 (d, 1H); 7.72(s, 1H); 7.52-7.65(m, 3H); 7.39-7.47 (m, 1H); 7.25(d, 2H); 7.17(d, 2H); 7.06(s, 1H); 6.93(s, 1H); 3.28-3.40 (m, 2H); 2.82-1.96(m, 2H); 1.60-1.86(m, 2H); 1.32-1.48(m, 2H). | | | |
| #MS (ESI): [M + H]⁺ = 603 (Cl isotope pattern well displayed). | | | |
| 1.11 | 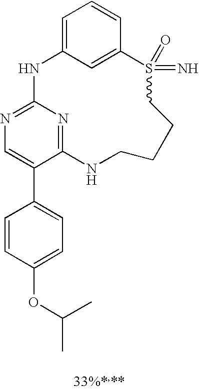 | (RS)-4-Imino-1⁵-[4- (1-methylethoxy)phenyl]- 4-thia-2,9-diaza- 1(2,4)-pyrimidina- 3(1,3)- benzenacyclononaphane 4-oxide | |
| #¹H-NMR (DMSO): 9.37(s, 1H); 8.51(s, 1H); 7.70(s, 1H); 7.34-7.47(m, 2H); 7.18-7.30(m app d, 3H); 6.95(d, 2H); 6.68(t br, 1H); 4.60(sept, 1H); 4.12 (s, 1H); 3.24(mc, 2H); 2.90-3.07(m, 2H); 1.43-1.93(m, 4H); 1.25(d, 6H). MS (ESI): [M + H]⁺ = 438. | | | |
| 1.12 | 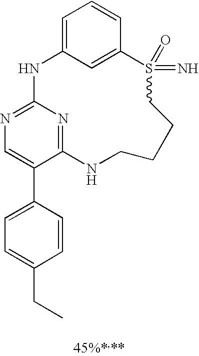 | (RS)-1⁵-(4- Ethylphenyl)-4- imino-4-thia-2,9- diaza-1(2,4)- pyrimidina-3(1,3)- benzenacyclononaphane 4-oxide | |
| #¹H-NMR (DMSO): 9.44(s, 1H); 8.55(5, 1H); 7.76(s, 1H); 7.38-7.51(m, 2H); 7.22-7.34(m, 5H); 6.80 (t br, 1H); 4.17(s, 1H); 3.31(mc, 2H); 2.97-3.12 (m, 2H); 2.66(q, 2H); 1.49-1.96(m, 4H); 1.22 (t, 3H). MS (ESI): [M + H]⁺ = 408. | | | |
| 1.13 | 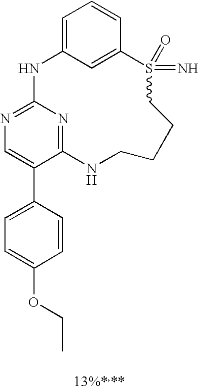 | (RS)-1⁵-(4- Ethoxyphenyl)-4- imino-4-thia-2,9- diaza-1(2,4)- pyrimidina-3(1,3)- benzenacyclononaphane 4-oxide | |
| #¹H-NMR (DMSO): 9.39(s, 1H); 8.51(s, 1H); 7.70(s, 1H); 7.33-7.45(m, 2H); 7.18-7.28(m, 3H); 6.95 (d, 2H); 6.68(t br, 1H); 4.13(s, 1H); 4.02(q, 2H); 3.25(mc, 2H); 2.91-3.07 (m, 2H); 1.45-1.93(m, 4H); 1.32(t, 3H). MS (ESI): [M + H]⁺ = 424. | | | |
| 1.14 | 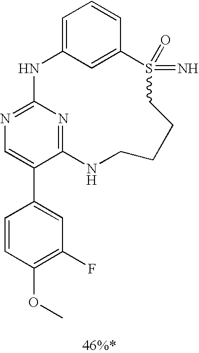 | (RS)-1⁵-(3-Fluoro-4- methoxyphenyl)-4- imino-4-thia-2,9- diaza-1(2,4)- pyrimidina-3(1,3)- benzenacyclononaphane 4-oxide | |
| #¹H-NMR (DMSO): 9.43(s, 1H); 8.49(s, 1H); 7.73(s, 1H); 7.34-7.46(m, 2H); 7.14-7.28(m, 3H); 7.09 (dd, 1H); 6.82(t br, 1H); 4.14(s, 1H); 3.82(s, 3H); 3.26(mc, 2H); 2.86-3.05 (m, 2H); 1.42-1.94(m, 4H). MS (ESI): [M + H]⁺ = 428. | | | |
| 1.15 | 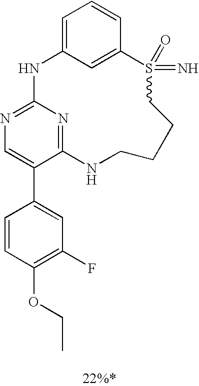 | (RS)-1⁵-(4-Ethoxy-3- fluorophenyl)-4- imino-4-thia-2,9- diaza-1(2,4)- pyrimidina-3(1,3)- benzenacyclononaphane 4-oxide | |
| #¹H-NMR (DMSO): 9.43(s, 1H); 8.48(s, 1H); 7.72(s, 1H); 7.33-7.47(m, 2H); 7.12-7.28(m, 3H); 7.07 (dd, 1H); 6.83(t br, 1H); 4.18(s br, 1H); 4.10(q, 2H); 3.26(mc, 2H); 2.90-3.06(m, 2H); 1.43-1.92 (m, 4H); 1.32(t, 3H). MS (ESI): [M + H]⁺ = 442. | | | |

*10 mol-% of POPd used
**2.5 eq of boronic acid used

### Example 1.16

### Preparation of (RS)—N-{4-[4-Imino-4-oxo-4-thia-2,9-diaza-1(2,4)-pyrimidina-3(1,3)-benzenacyclononaphan-1⁵-yl]phenyl}-1-phenylcyclopropanecarboxamide

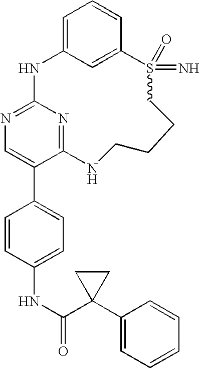

To a mixture of (RS)-1⁵-iodo-4-imino-4-thia-2,9-diaza-1(2,4)-pyrimidina-3(1,3)-benzenacyclononaphane 4-oxide (105 mg, 0.24 mmol) and 1-phenyl-N-[4-(4,4,5,5-tetramethyl-1,3,2-dioxaborolan-2-yl)phenyl]cyclopropanecarboxamide (124 mg, 0.34 mmol; intermediate 33) in ethanol (5.1 mL) and toluene (5.1 mL) was added a 1M aqueous solution of potassium carbonate (0.65 mL), lithium chloride (29 mg, 0.69 mmol) and tetrakis-triphenylphosphinato-palldium (0) (24 mg, 0.02 mmol) under an atmosphere of argon. The resulting mixture was stirred at 90° C. for 4 h. After cooling to room temperature, water (20 mL) was added, followed by extraction with ethyl acetate (3×20 mL). The combined organic layers were washed with water and brine (15 mL each), dried over magnesium sulfate, and evaporated. The crude product was purified by column chromatography on silica, followed by trituration with methanol (89 mg, 0.17 mmol, 67% yield).

¹H-NMR (DMSO, 300 MHz): 9.41 (s, 1 H); 9.19 (s, 1 H); 8.47 (s br, 1 H); 7.78 (s, 1 H); 7.61 (d, 2 H); 7.17-7.46 (m, 10 H); 6.72 (t br, 1 H); 4.13 (s, 1 H); 3.21-3.35 (m, 2 H); 2.88-3.03 (m, 2 H); 1.44-1.92 (m, 4 H); 1.40 (mc, 2 H); 1.09 (mc, 2 H).

MS (ESI): [M+H]⁺=539.

### Example 1.17

### Preparation of (RS)-2,3-Dichloro-N-{4-[4-imino-4-oxo-4-thia-2,9-diaza-1(2,4)-pyrimidina-3(1,3)-benzenacyclononaphan-1⁵-yl]but-3-ynyl}benzenesulfonamide

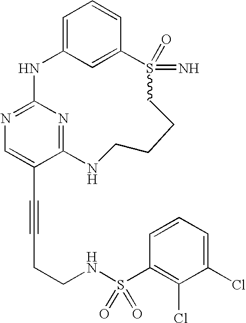

To a degassed suspension of (RS)-1⁵-iodo-4-imino-4-thia-2,9-diaza-1(2,4)-pyrimidina-3(1,3)-benzenacyclononaphan 4-oxide (322 mg, 0.75 mmol), N-butynyl-2,3-dichlorobenzene sulfonamide (417 mg, 1.50 mmol, intermediate 34), copper (I) iodide (14 mg, 75 μmol), and palladium dichlorobis(triphenylphosphine) (26 mg, 0.038 mmol) in DMF (6.0 mL) was added TEA (1.04 mL, 7.5 mmol) under an atmosphere of argon over a period of 5 min at room temperature, and the resulting mixture was then stirred for 4 h. Water (20 mL) was added, followed by extraction with ethyl acetate (5×40 mL). The combined organic layers were washed with brine (20 mL), dried over magnesium sulfate, and evaporated. The crude residue was triturated with acetonitrile to give the desired product (295 mg, 0.45 mmol, 68% yield).

¹H-NMR (DMSO, 300 MHz): 9.57 (s br, 1 H); 8.41 (s br, 1 H); 8.31 (t, 1 H); 7.80-8.02 (m, 3 H); 7.51 (t, 1 H); 7.36-7.47 (m, 2 H); 7.17-7.28 (m, 1 H); 7.03 (t br, 1 H); 4.17 (s br, 1 H); 3.22-3.40 (m, 2 H, partly covered by water peak); 2.93-3.18 (m, 4 H); 2.53 (t, 2 H); 1.47-1.92 (m, 4 H).

MS (ESI): [M+H]⁺=579 (Cl isotopes well displayed).

### Example 1.18

### Preparation of 2-{4-[(RS)-4-Imino-4-oxo-4λ⁶-thia-2,9-diaza-1(2,4)-pyrimidina-3(1,3)-benzenacyclononaphan-1⁵-yl]phenoxy}-1-(2,4-xylyl)ethan-1-one

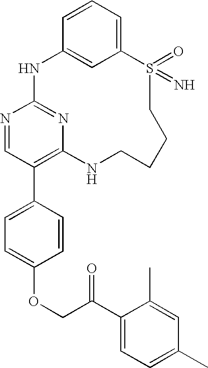

A solution of benzyl N—[(RS)-1⁵-{4-[(2,4-dimethylbenzoyl)methoxy]phenyl}-4-oxo-4λ⁶-thia-2,9-diaza-1(2,4)-pyrimidina-3(1,3)-benzenacyclononaphan-4-ylidene]carbamate (15 mg, 0.022 mmol; intermediate 23) in ethanol (9 mL) and N,N-dimethylacetamide (2 mL) was treated with a 10% palladium on charcoal catalyst (2.4 mg, 2.2 μmol) and stirred at room temperature under an atmosphere of hydrogen for 12 h. The catalyst was filtered off, the solvent was evaporated and the residue was purified by HPLC to give the desired compound (2.4 mg, 4.4 μmol, 20% yield).

¹H-NMR (DMSO, 300 MHz): 9.71 (s br, 1 H); 8.46 (s br, 1 H); 7.88 (d, 1 H); 7.74 (s, 1 H); 7.45-7.55 (m, 2 H); 7.24-7.36 (m, 3 H); 7.13-7.24 (m, 2 H); 7.03 (d, 2 H); 5.43 (s, 2 H); 3.30-3.45 (m, 2 H, covered by water peak); 2.93-3.08 (m, 2 H); 2.43 (s, 3 H); 2.33 (s, 3 H); 1.71-1.92 (m, 2 H); 1.43-1.68 (m, 2 H).

MS (ESI): [M+H]⁺=542.

### Example 1.19

### Preparation of (RS)-4-Imino-1⁵-iodo-3⁵-nitro-4-thia-2,9-diaza-1(2,4)-pyrimidina-3 (1,3)-benzenacyclononaphane 4-oxide

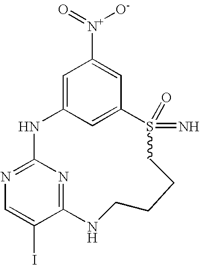

To a mixture of acetonitrile (168 mL), water (18.6 mL), and 4N hydrogen chloride in 1,4-dioxane (1.86 mL, 7.43 mmol) was added a solution of (RS)—S-(3-amino-5-nitrophenyl)-S-{4-[(5-iodo-2-chloropyrimidin-4-yl)amino]butyl}-sulfoximide (600 mg; 1.17 mmol; intermediate 17) in acetonitrile (100 mL) over a period of 2 h at a temperature of 50° C. The mixture was stirred overnight at 50° C. and then for 5 h at 90° C. and finally for another 24 h at 50° C. After cooling to room temperature, the mixture was concentrated to a volume of approx. 15 mL, and aqueous sodium bicarbonate (10 mL) was added. The precipitate was isolated by filtration, washed with water, and dried. The crude product was heated with acetonitrile (10 mL), filtered off from the insolubles whilst hot, and evaporated, followed by purification by column chromatography and HPLC to give the title compound (8.4 mg, 18 μmol, 2% yield).

¹H-NMR (DMSO, 300 MHz): 10.16 (s, 1 H); 8.84-8.92 (m, 1 H); 8.20 (s, 1 H); 8.14-8.18 (m, 1 H); 8.07-8.11 (m, 1 H); 7.47 (s br, 1 H); 3.44 (mc, 2 H); 2.98-3.14 (m, 2 H); 1.49-1.84 (m, 4 H).

MS (ESI): [M+H]⁺=475.

### Example 1.20

### Preparation of N—[(RS)-4-Imino-1⁵-iodo-4-oxo-4λ⁶-thia-2,9-diaza-1(2,4)-pyrimidina-3(1,3)-benzenacyclononaphan-3⁵-yl]pyrrolidine-1-carboxamide

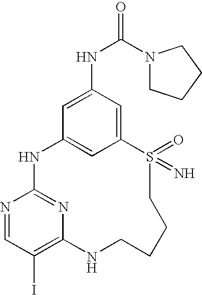

A solution of N—[(RS)-1⁵-iodo-4-oxo-4-({[2-(trimethylsilyl)ethyl]sulfonyl}imino)-4⁶-thia-2,9-diaza-1(2,4)-pyrimidina-3(1,3)-benzenacyclononaphan-3⁵-yl]-pyrrolidine-1-carboxamide (42 mg, 0.06 mmol; intermediate 31) in THF (3 mL) was treated with a 1N solution of tetra-n-butylammonium fluoride (0.36 mL, 0.36 mmol) and stirred under reflux for 5 h. After cooling to room temperature, ethyl acetate was added and the mixture was extracted with aqueous sodium bicarbonate and brine. The organic layer was dried and evaporated, and the crude product was purified by column chromatography to give the desired free sulfoximine (22 mg, 41 μmol, 67% yield).

¹H-NMR (DMSO, 400 MHz): 9.41 (s, 1 H); 8.42 (s, 1 H); 8.08 (s, 1 H); 7.82 (s, 1 H); 7.62 (s, 1 H); 7.54 (s, 1 H); 6.89 (s br, 1 H); 4.03 (s, 1 H); 3.08-3.46 (m, 6 H); 2.88-3.05 (m, 2 H); 1.38-1.90 (m, 8 H).

MS (ESI): [M+H]⁺=542.

### Example 1.21

### Preparation of N—[(RS)-1⁵-(4-Ethoxyphenyl)-4-imino-4-oxo-4λ⁶-thia-2,9-diaza-1(2,4)-pyrimidina-3(1,3)-benzenacyclononaphan-3⁵-yl]pyrrolidine-1-carboxamide

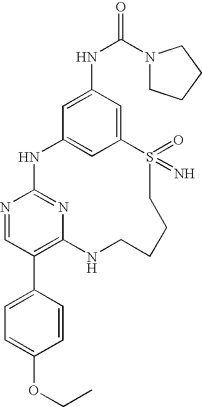

A solution of N—[(RS)-1⁵-(4-Ethoxyphenyl)-4-oxo-4-({[2-(trimethylsilyl)ethyl]sulfonyl}imino)-4λ⁶-thia-2,9-diaza-1(2,4)-pyrimidina-3(1,3)-benzenacyclononaphan-3⁵-yl]pyrrolidine-1-carboxamide (17 mg, 0.024 mmol; intermediate 32) in THF (4 mL) was treated with a 1N solution of tetra-n-butylammonium fluoride (73 μL, 0.073 mmol) and stirred at 50° C. for 2.5 h. Subsequently, another portion of tetra-n-butylammonium fluoride (73 μL, 0.073 mmol) was added and the mixture was refluxed for another 3 h. After cooling to room temperature, ethyl acetate was added and the mixture was extracted with aqueous sodium bicarbonate and brine. The organic layer was dried and evaporated, and the crude product was purified by column chromatography to give the desired free sulfoximine (7.3 mg, 14 μmol, 56% yield).

¹H-NMR (DMSO, 300 MHz):9.33 (s, 1 H); 8.42 (s, 1 H); 7.98-8.04 (m, 1 H); 7.72 (s, 1 H); 7.62 (s, 1 H); 7.56 (s, 1 H); 7.28 (d, 2 H); 7.01 (d, 2 H); 6.68 (t br, 1 H); 3.99-4.14 (m, 3 H); 3.16-3.48 (m, 6 H); 2.91-3.07 (m, 2 H); 1.43-1.95 (m, 8 H); 1.36 (t, 3 H).

MS (ESI): [M+H]⁺=535.

The following Example compounds may be obtained using the methods described hereinbefore and/or by standard procedures known to the person skilled in the art

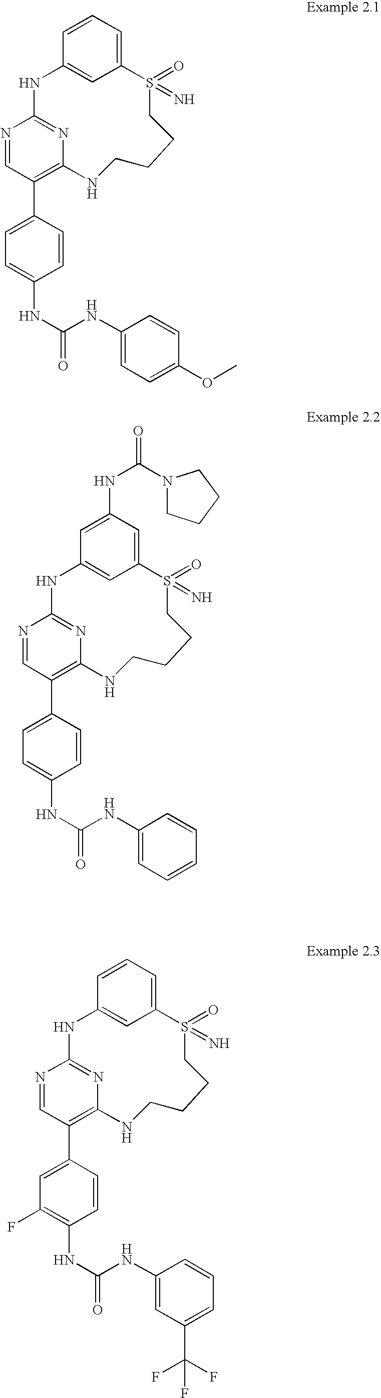

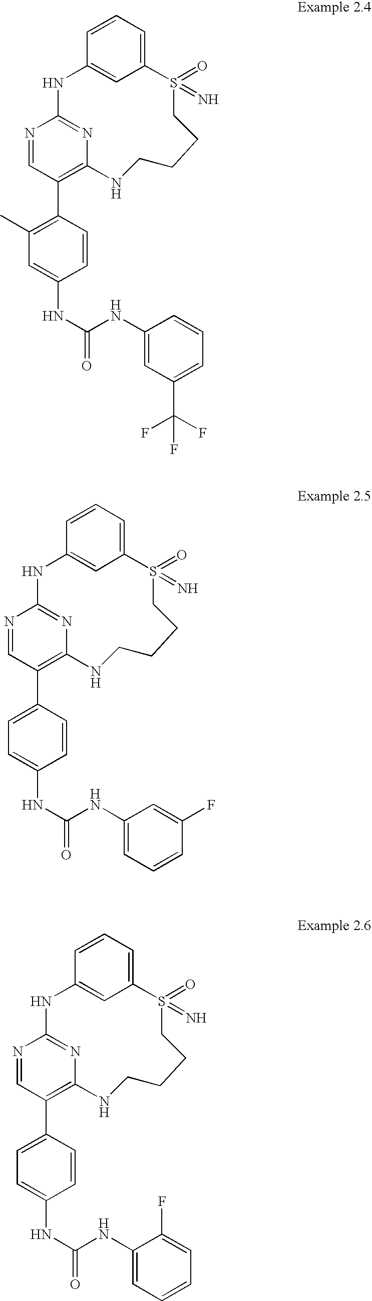

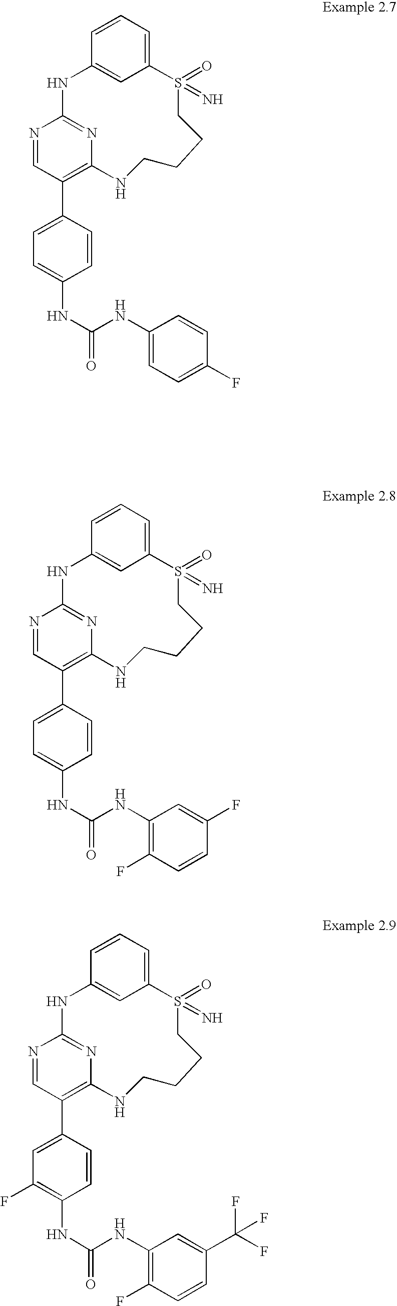

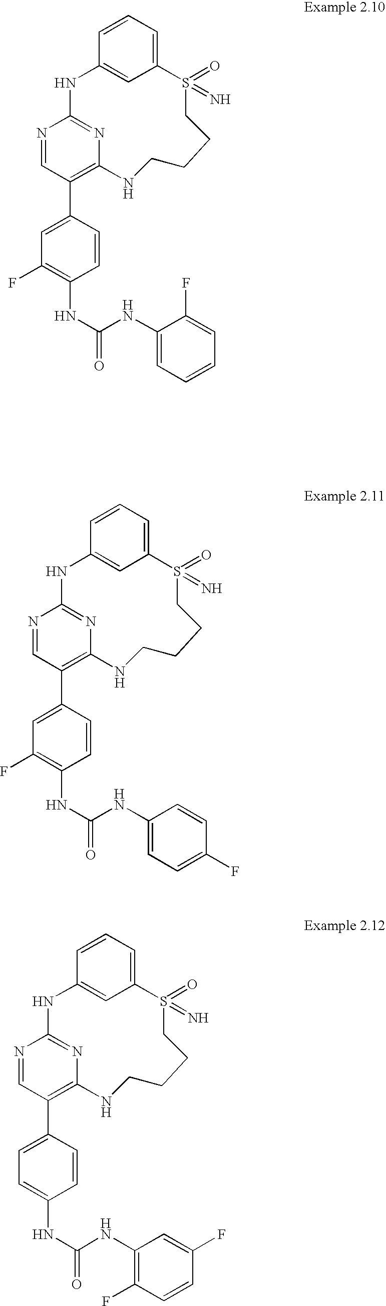

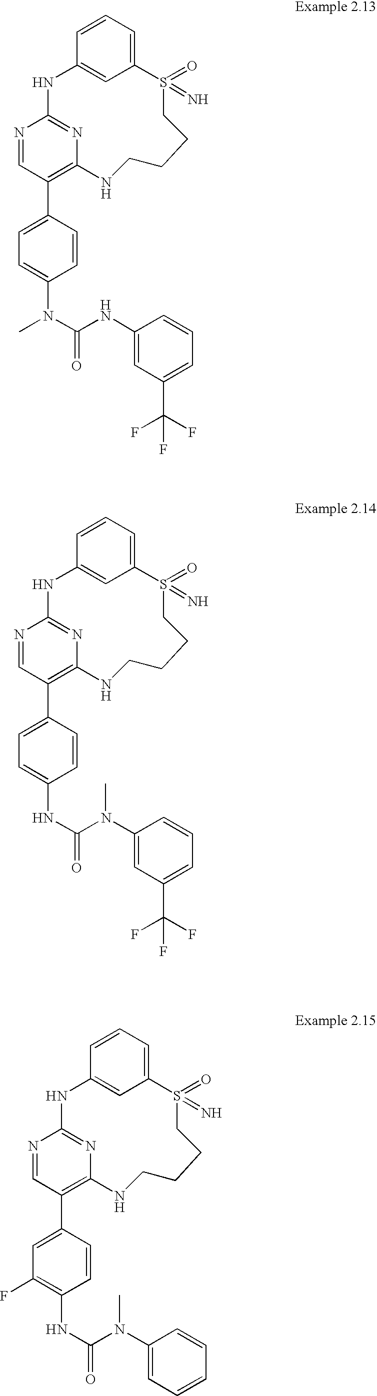

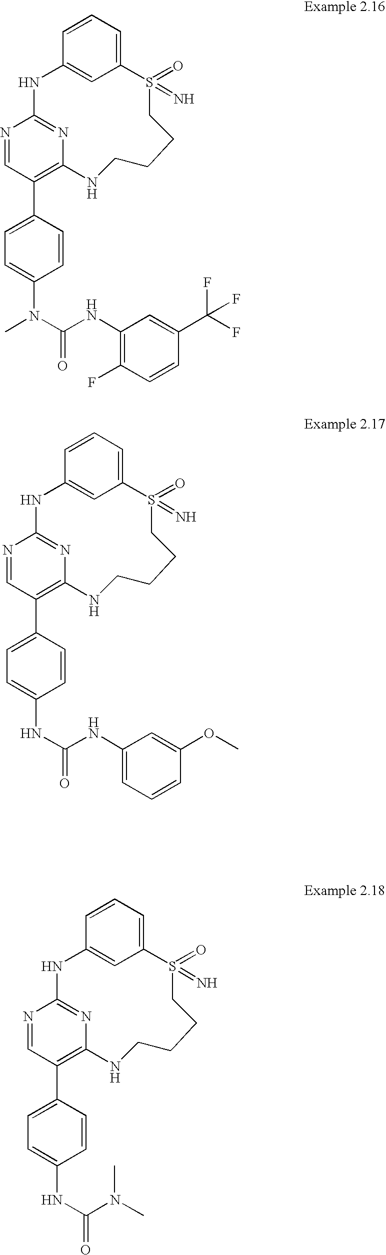

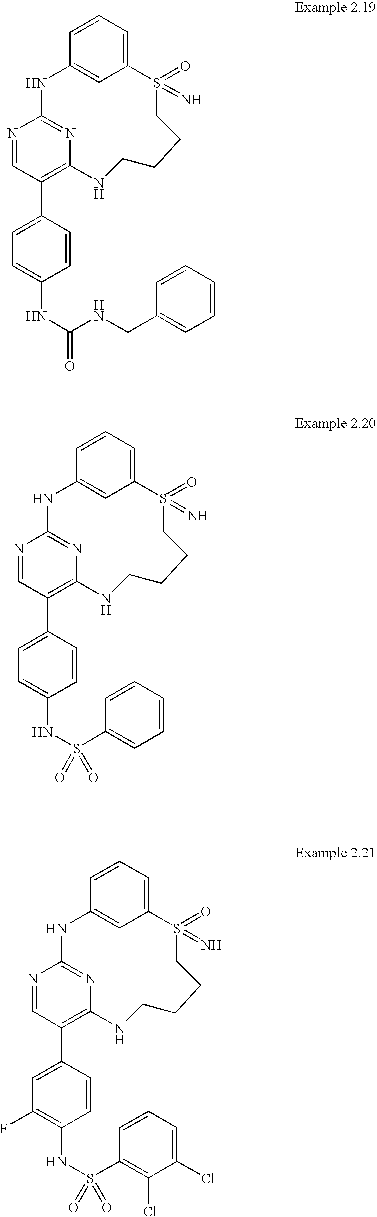

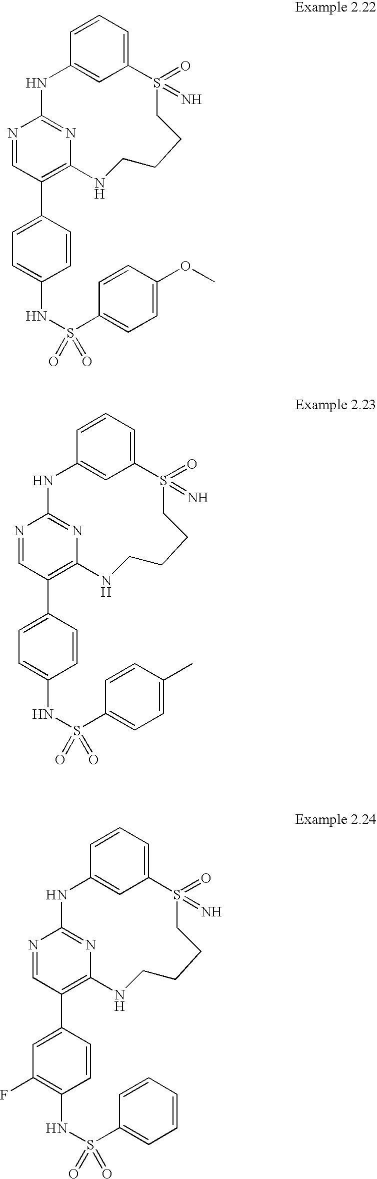

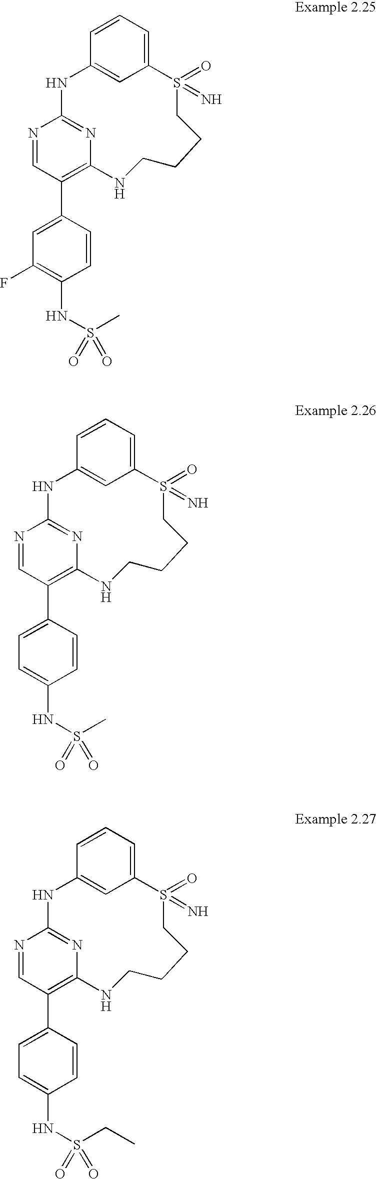

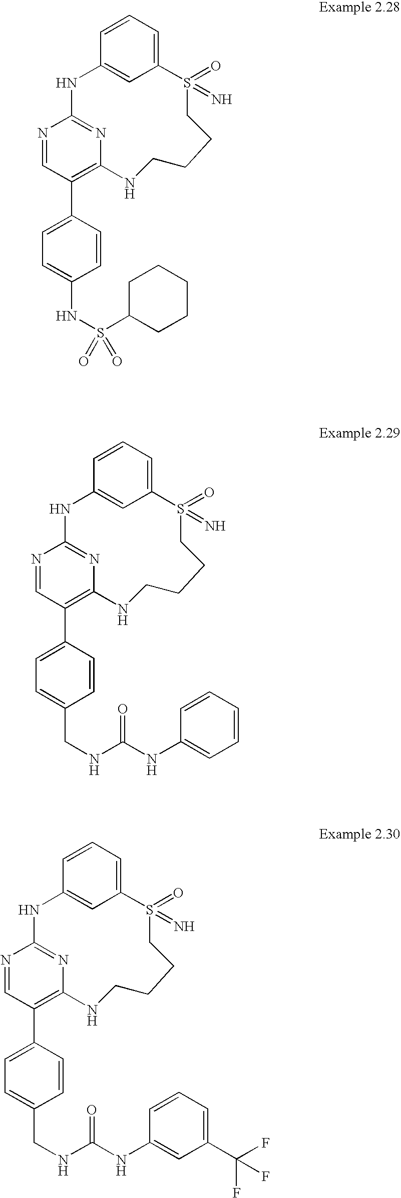

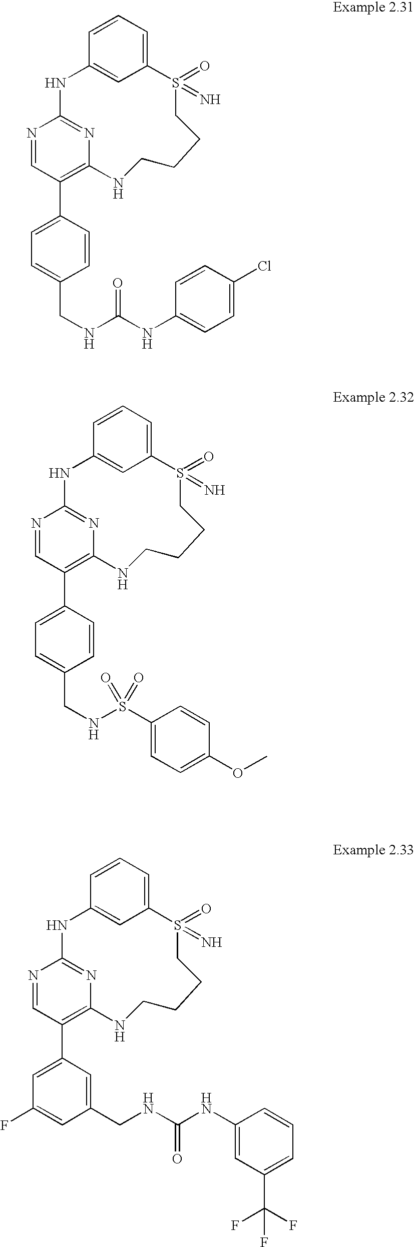

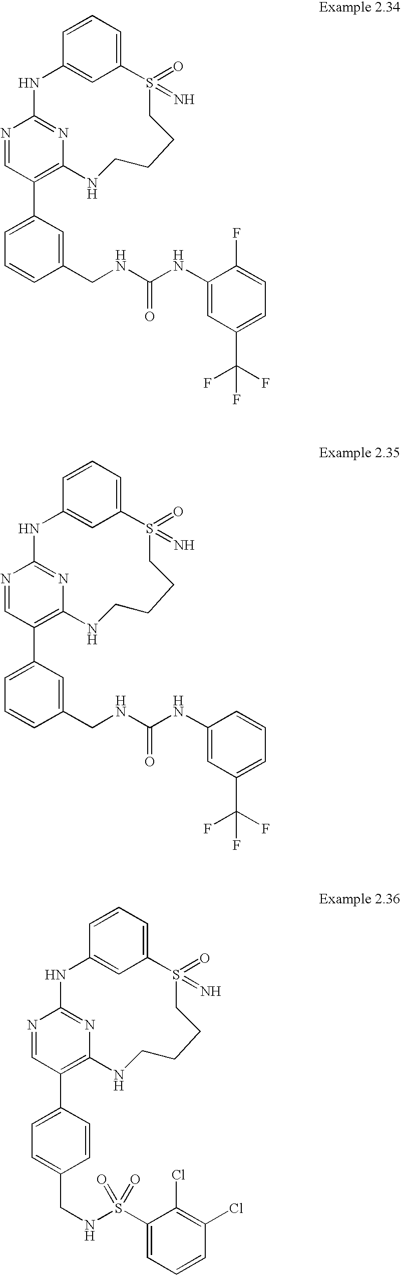

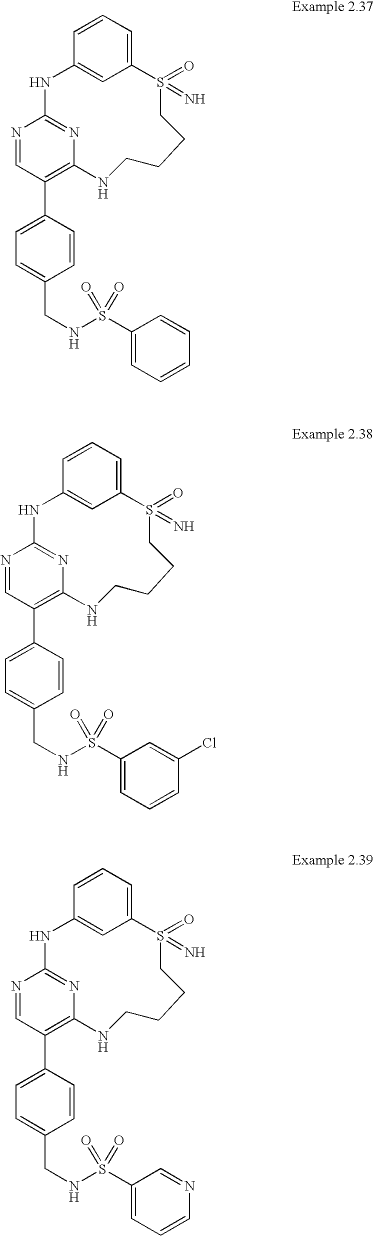

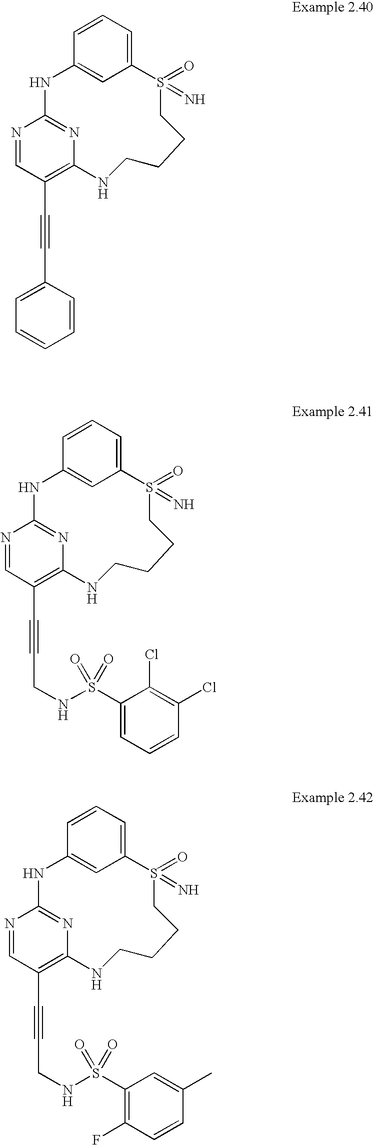

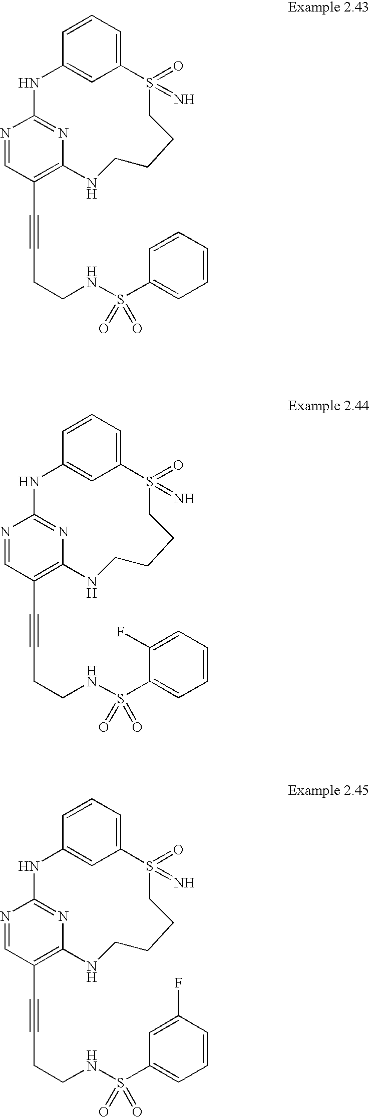

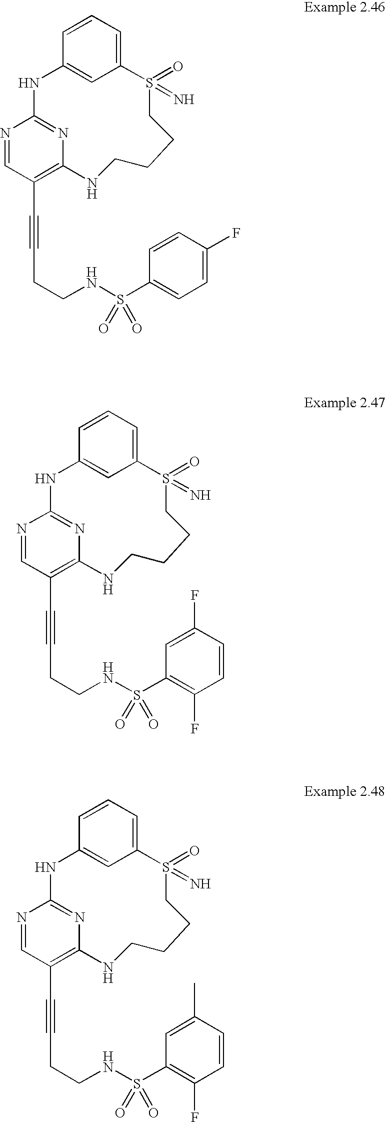

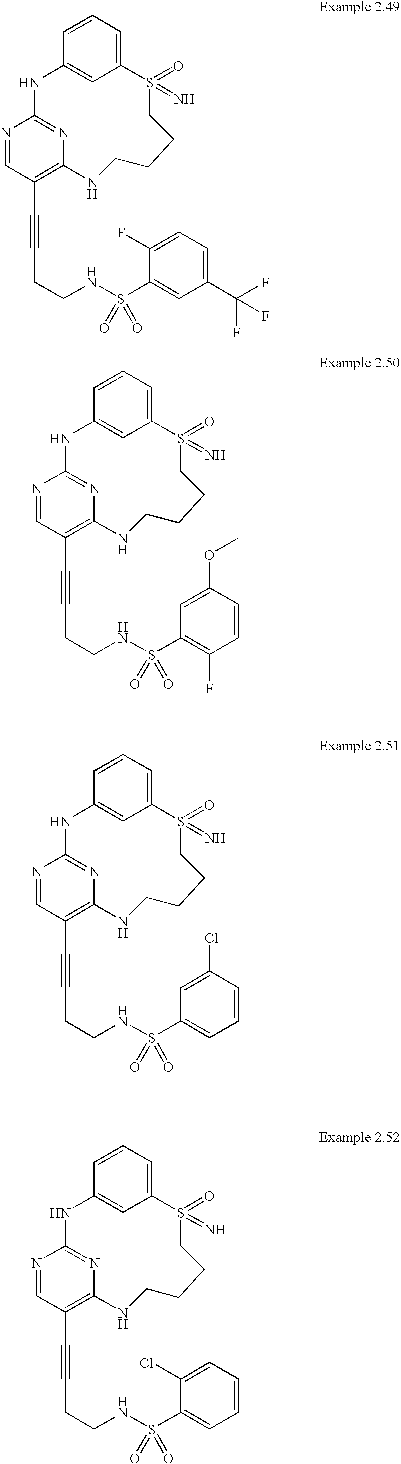

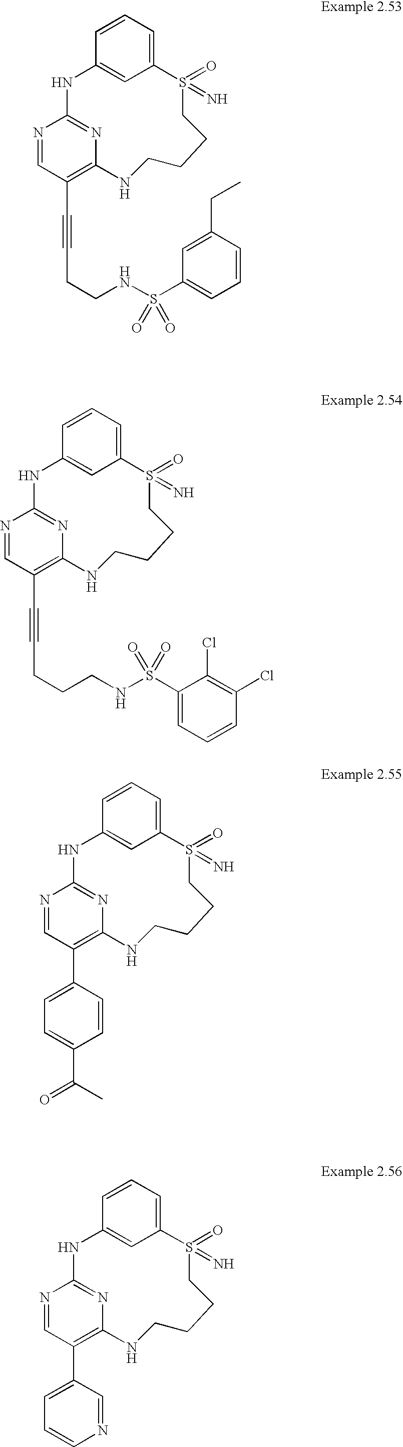

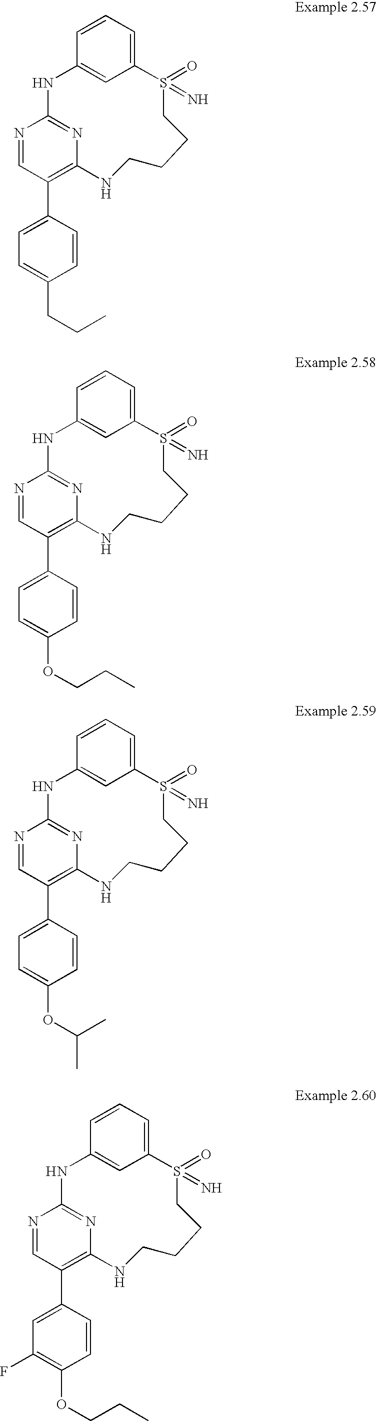

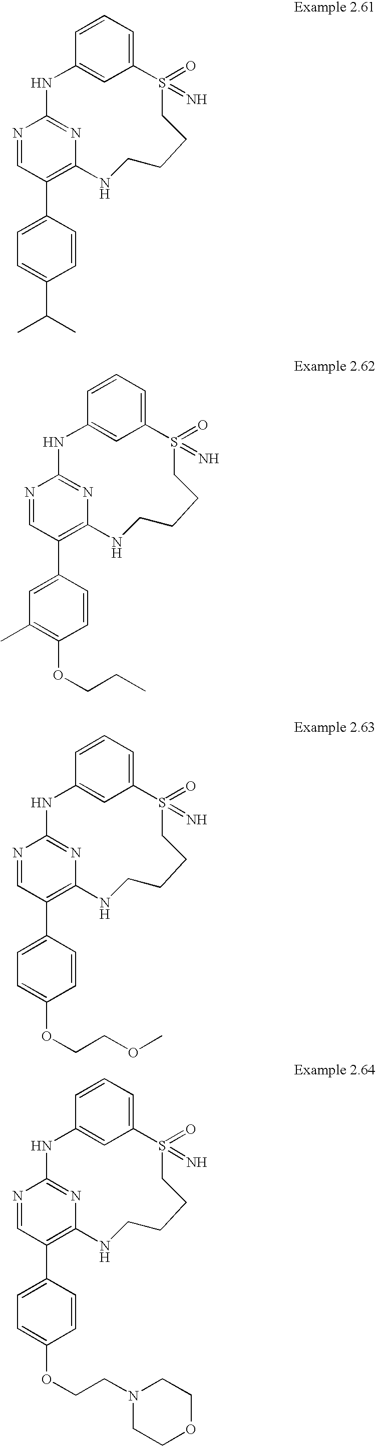

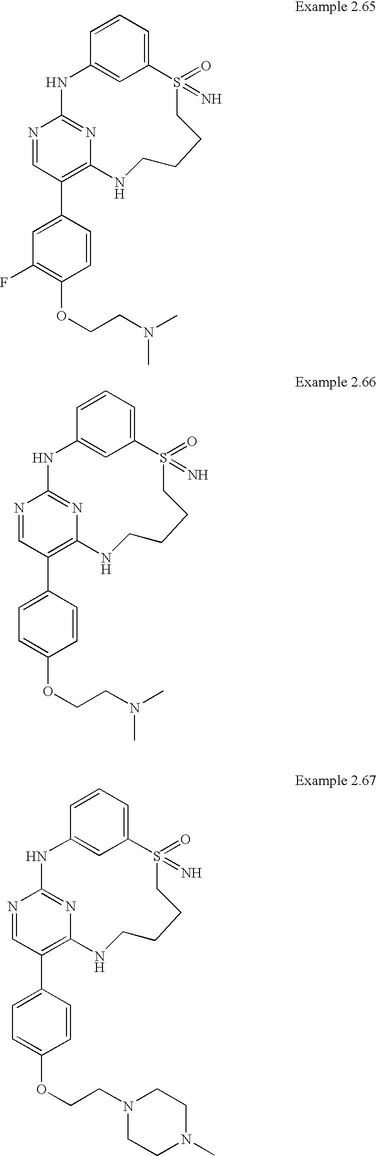

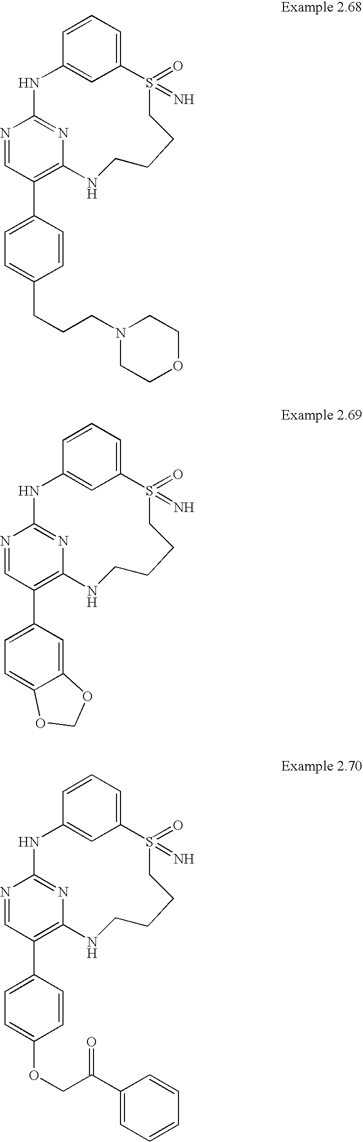

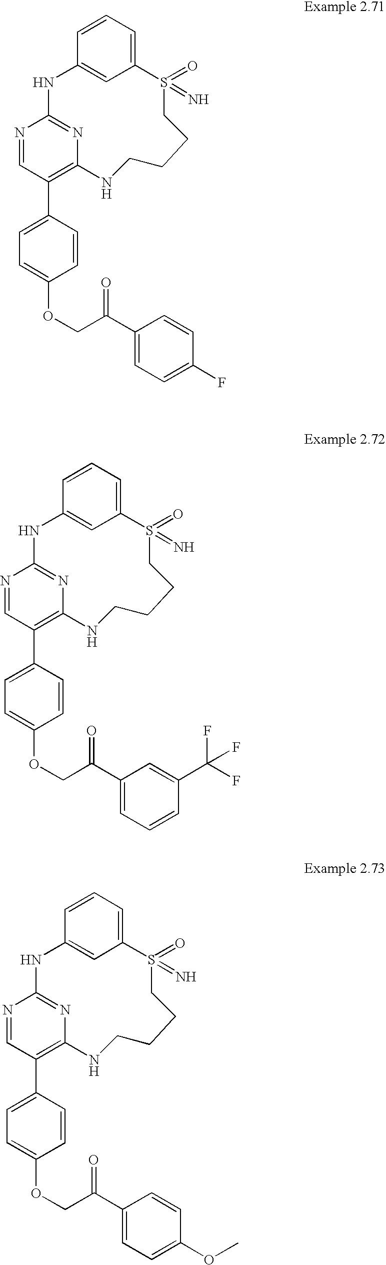

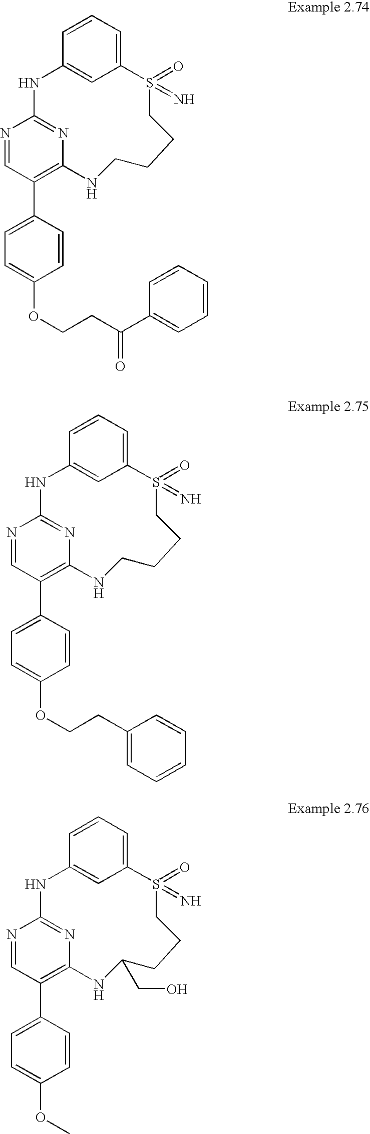

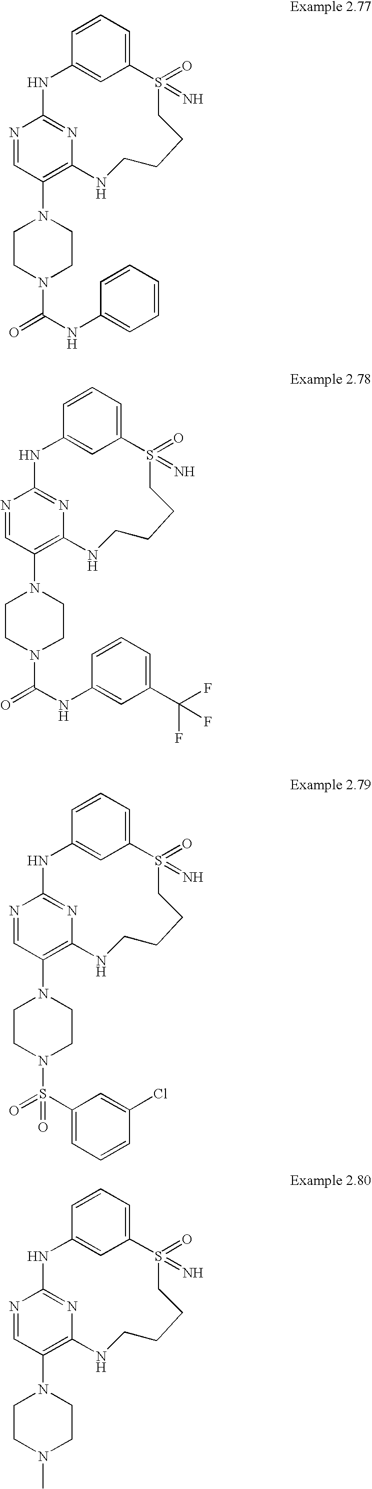

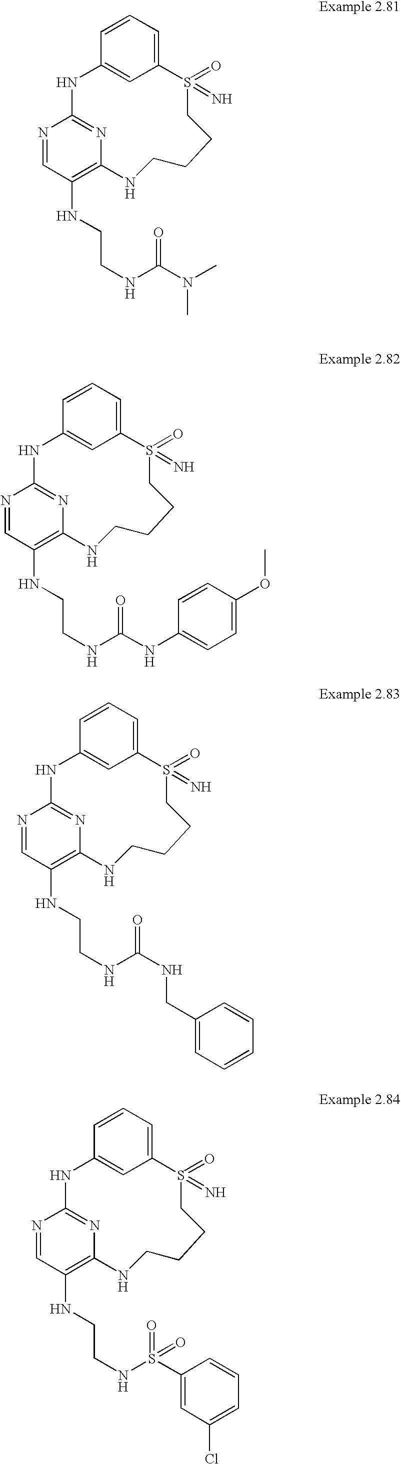

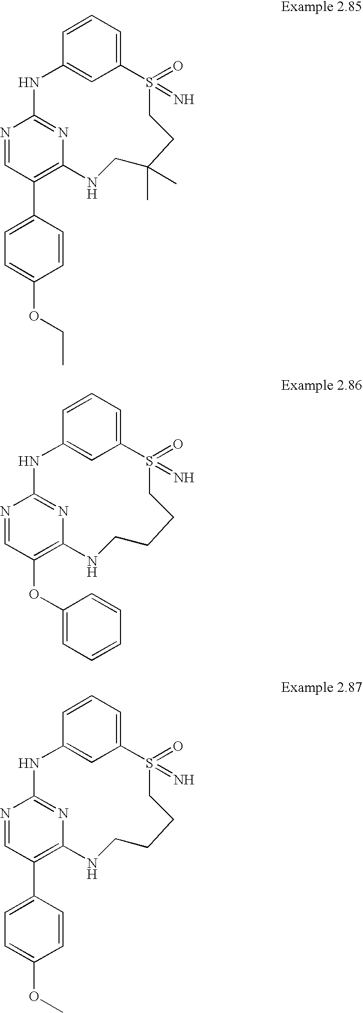

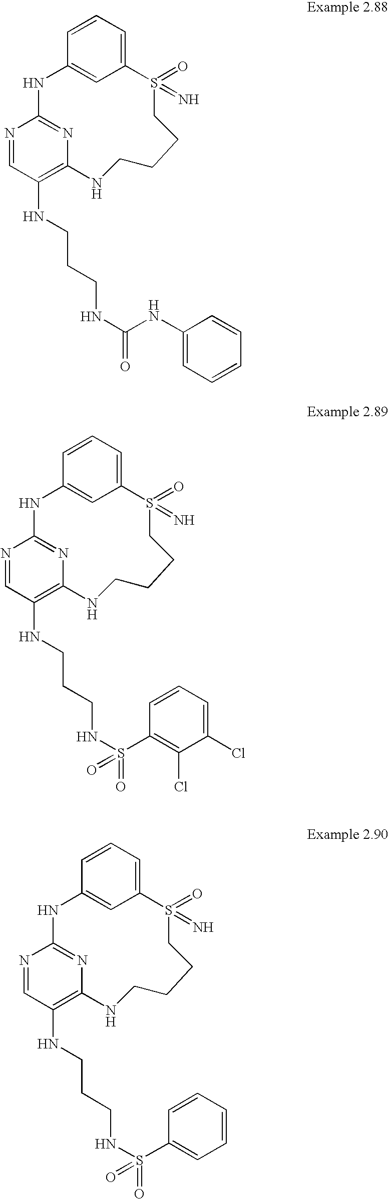

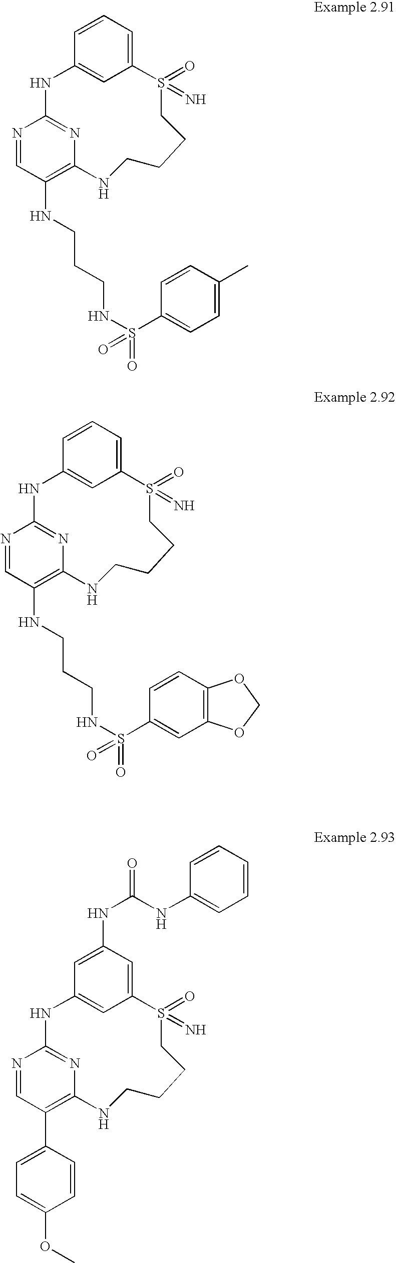

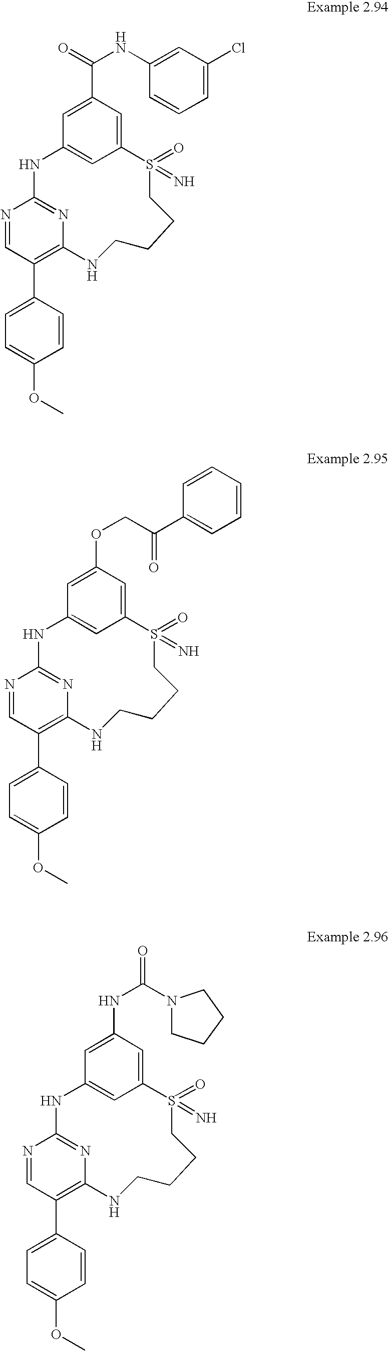

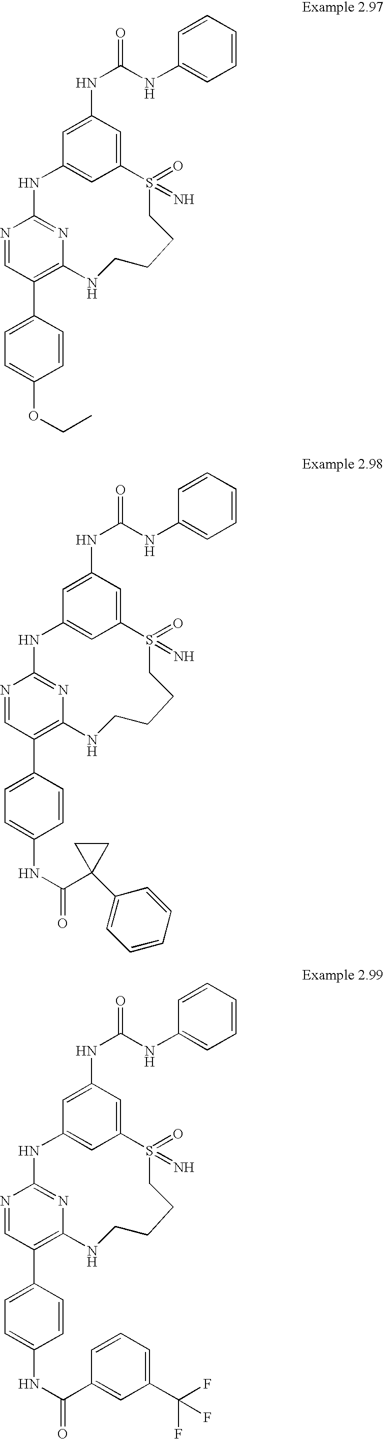

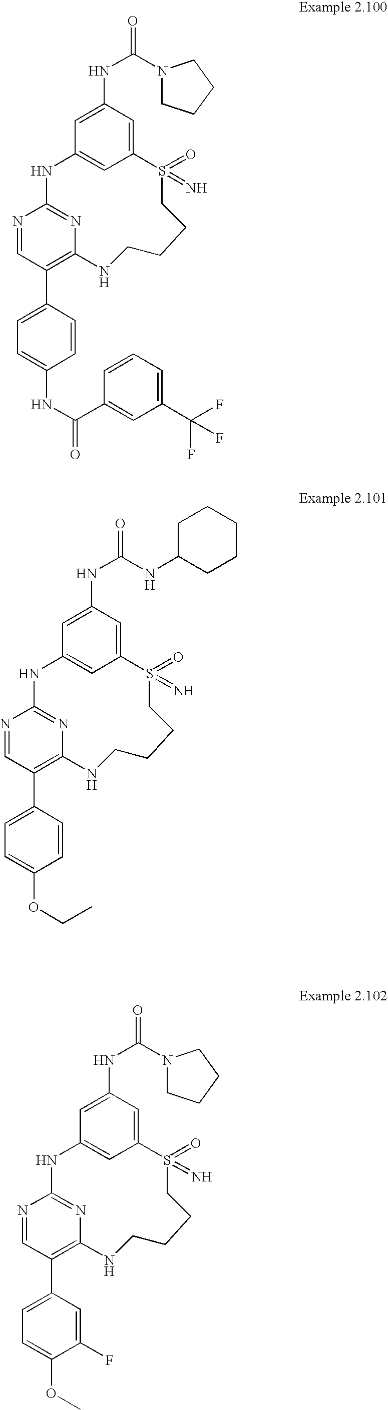

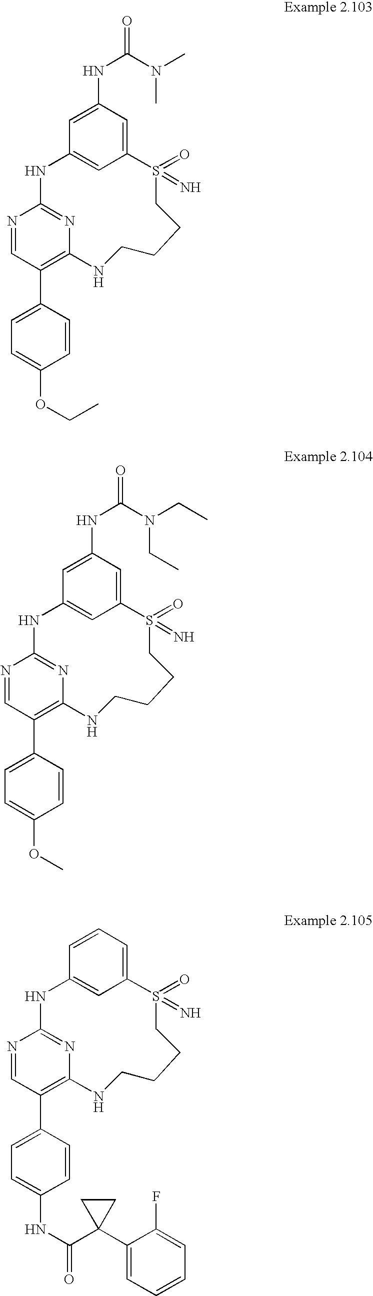

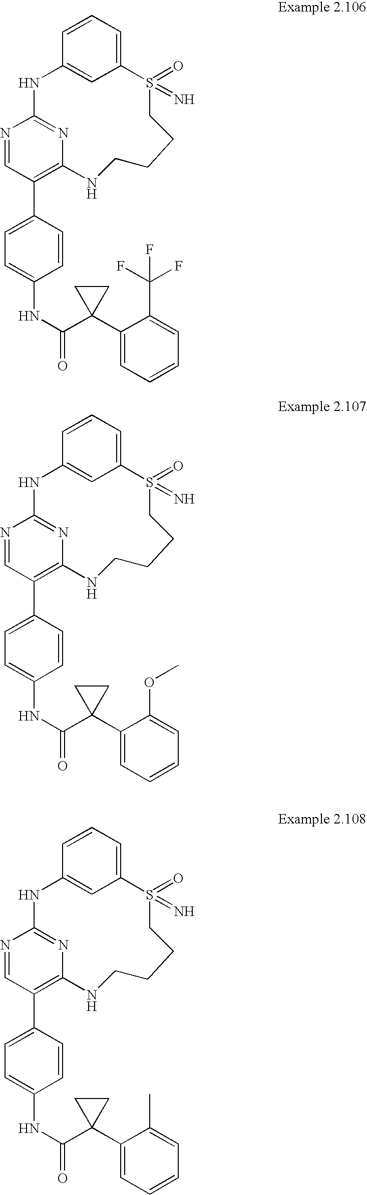

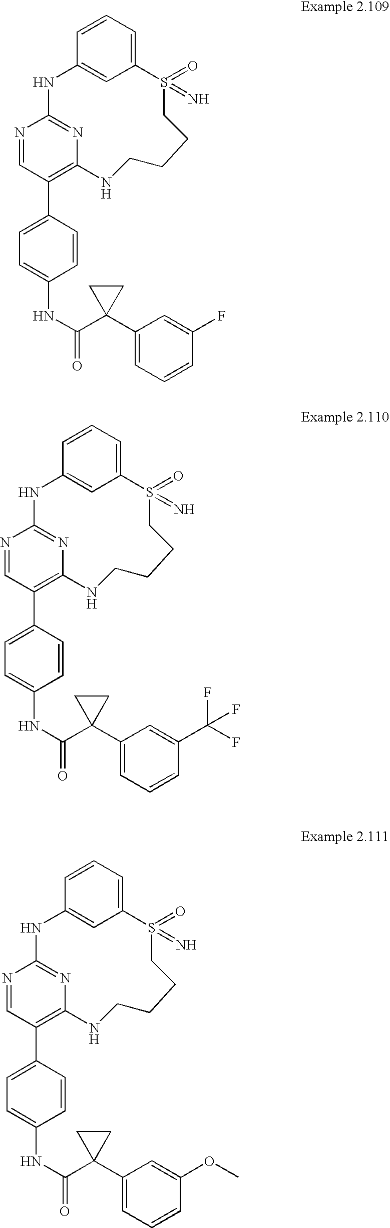

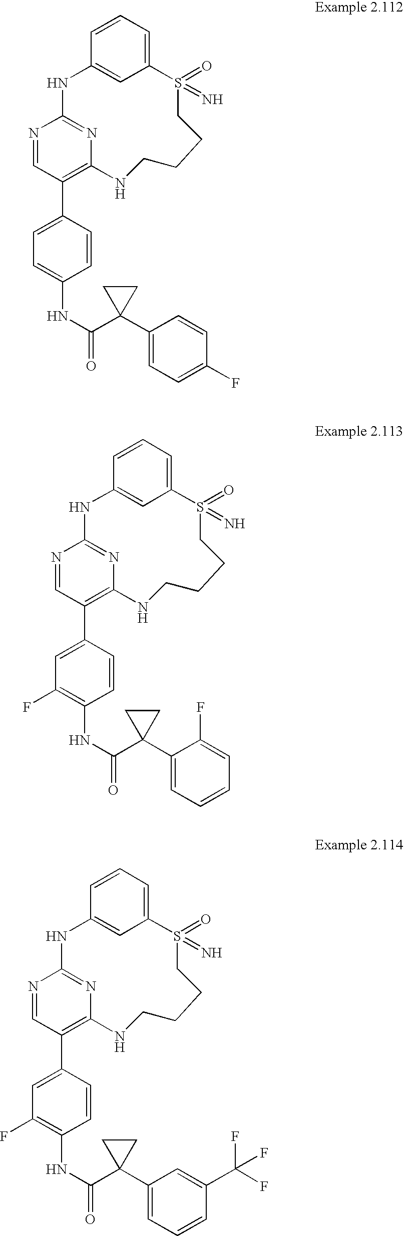

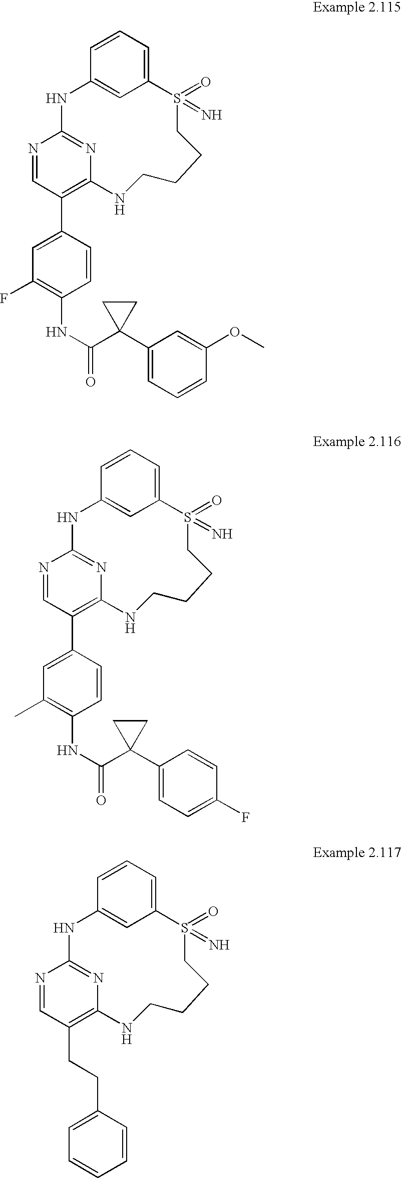

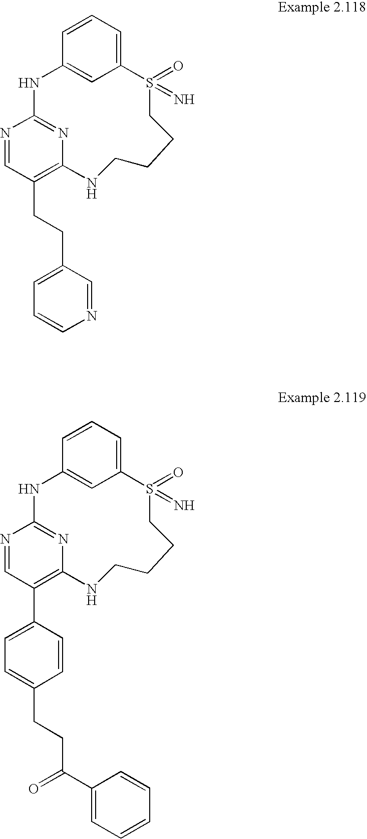

### Biological Experiment 1: ELISA Method

To prove the high potency activity as inhibitors of Tie2 kinase and Tie2 autophosphorylation the following ELISA-method was established and used.

Herein CHO cell-cultures, which are stably transfected by known techniques with Tie2 using DHFR deficiency as selection marker, are stimulated by angiopoietin-2. The specific autophosphorylation of Tie2 receptors is quantified with a sandwich-ELISA using anti-Tie2 antibodies for catch and anti-phosphotyrosine antibodies coupled to HRP as detection.

Materials
96well tissue culture plate, sterile, Greiner
96well FluoroNunc plate MaxiSorp Surface C, Nunc
96well plate polypropylene for compound dilution id DMSO
CHO Tie2/DHFR (transfected cells)
PBS−; PBS++, DMSO
MEM alpha Medium with Glutamax-I without Ribonucleosides and Deoxyribonucleosides (Gibco #32561-029)
   with 10% FCS after dialysis! and 1% PenStrep
Lysis buffer: 1 Tablet “Complete” protease inhibitor
   1 cap Vanadate (1 mL>40 mg/mL; working solution 2 mM)
   ad 50 mL with Duschl-Puffer
   pH 7.6
Anti-TIE-II antibody 1:425 in Coating Buffer pH 9.6
   Stock solution: 1.275 mg/mL >working.: 3 μg/mL
PBST: 2 bottles PBS(10×)+10 mL Tween, fill up with VE-water
RotiBlock 1:10 in VE-water
Anti-Phosphotyrosine HRP-Conjugated 1:10000 in 3% TopBlock
   3% TopBlock in PBST
BM Chemiluminescence ELISA Substrate (POD)
   solution B 1:100 solution A
SF9 cell culture medium
Ang2-Fc in SF9 cell culture medium

Cell Experiment
Dispense 5×10⁴ cells/well/98 μL in 96well tissue culture plate
Incubate at 37° C./5% CO₂
After 24 h add compounds according to desired concentrations
Add also to control and stimulated values without compounds 2 μL DMSO
And mix for a few min at room temperature
Add 100 μL Ang2-Fc to all wells except control, which receives insect medium
Incubate 20 min at 37° C.
Wash 3× with PBS++
Add 100 μl Lysis buffer/well and shake a couple of min at room temperature
Store lysates at 20° C. before utilizing for the ELISA

Performance of Sandwich-ELISA
Coat 96well FluoroNunc Plate MaxiSorp Surface C with anti-Tie2 Mab
   1:425 in Coating buffer pH 9.6; 100 μL/well overnight at 4° C.
Wash 2× with PBST
Blcock plates with 250 μL/well RotiBlock 1:10 in VE-water
Incubate for 2 h at room temperature or overnight at 4° C. shaking
Wash 2× in PBST
Add thawed lysates to wells and incubate overnight shaking at 4° C.
Wash 2× with PBST
Add 100 μL/well anti-Phosphotyrosine HRP-Conjugated 1:10000 in 3% TopBlock (3% TopBlock in PBST) and incubate overnight under shaking
Wash 6× with PBST
Add 100 μL/well BM Chemiluminescence ELISA Substrate (POD) solutions 1 und 2 (1:100)
Determine luminescence with the LumiCount.

Biological Experiment 2: Tie-2-Kinase HTRF-Assay

To prove the effectiveness of the compound according to the present invention a Tie-2-Kinase HTRF-Assay was established.

Tie-2 phosphorylates tyrosine residues of the artificial substrate polyGAT (biotinylated polyGluAlaTyr). Detection of phosphorylated product is achieved specifically by a trimeric detection complex consisting of the phosphorylated substrate, streptavidin-XLent (SA-XLent) which binds to biotin, and Europium Cryptate-labeled anti-phosphotyrosine antibody PT66 which binds to phosphorylated tyrosine. Excitation of Europium fluorescence with 337 nm light results in emission of long-lived light with 620 nm. In case a trimeric detection complex has formed, part of the energy will be transferred to the SA-XLent fluorophore that itself then emits long-lived light of 665 nm (FRET: fluorescence resonance energy transfer). Unphosphorylated substrate does not give rise to light emission at 665 nm, because no FRET-competent trimeric detection complex can be formed. Measurement is performed in a Packard Discovery or BMG Rubystar instrument. A-counts (emission at 665 nm) will be divided by B-counts (emission at 620 nm) and multiplicated with a factor of 10000. The resulting numbers are called the “well ratio” of the sample.

Material:
Enzyme: Tie-2-Kinase, in house, aliquots (12×10 mL) stored at −80° C.
Substrate: PolyGAT labeled with Biotin (1000 μg/mL); CIS Bio; #61GATBLB; aliquots stored at −20° C.
ATP: Amersham Pharmacia Biotech Inc. #27-2056-01; 100 mM; stored at −20° C.
Antibody: PT66-Eu Cryptate; CIS Bio; #61T66KLB; 30 μg/mL; aliquots stored at −20° C.
SA-XLent; CIS Bio; #611 SAXLB; 1000 μg/mL; aliquots stored at −80° C.
Microplates: 384 Well black, SV, Greiner, #784076

Solutions:

Assay buffer:
50 mM HEPES (pH 7.0), 25 mM MgCl₂, 5 mM MnCl₂, 1 mM DTT, 0.5 mM Na₃VO₄, 0.01% (v/v) NP40, 1× Complete EDTA free

Enzyme working solution:
Tie-2 stock solution is diluted 1:250 in assay buffer

Substrate working solution:

PolyGAT (1000 μg/mL; 36.23 μM) is diluted 1:90.6 to 400 nM or 77.3 ng/well, ATP (100 mM) is diluted 1:5000 to 20.0 μM. Both dilutions in assay buffer. Final assay concentrations: poly-GAT: 200 nM or 5.25 μg/mL, ATP: 10 μM (1× Km each).

Detection solution: 50 mM HEPES (pH 7.0), BSA 0.2%, 0.6 M KF, 200 mM EDTA, PT66-Europium Cryptate 2.5 ng/well, SA-XLent Cis Bio 90 ng/well.

Assay steps

All steps at 20° C.
1. 0.75 μL of compound solution in 30% (v/v) DMSO
2. add 7 μL of substrate working solution
3. add 7 μL of enzyme working solution
4. incubate 75 min (reaction volume: 14.75 μL)
5. add 8 μL of detection solution
6. incubate 180 min or over night at 4° C. (total volume: 22.75 μL)
7. measure HTRF in Packard Discovery or BMG Rubystar instrument (delay 50 μs, integrated time 400 μs)

Final concentrations (in 14.75 μL reaction volume):
Enzyme: unknown
polyGAT (1× Km): 200 nM (77.3 ng)
ATP (1× Km): 10 μM
DMSO: 1.5% (v/v)
Buffer conditions: 50 mM HEPES (pH 7.0), 25 mM MgCl₂, 5 mM MnCl₂, 1 mM DTT, 0.5 mM NaVO4, 0.01% (v/v) NP40, 1× Complete

Controls:
C₀: uninhibited reaction (DMSO only)
Cᵢ: inhibited reaction with 20 μM Staurosporine

Biological Experiment 3: Proliferation Test

To examine cell toxicity a cell proliferation test were established.

With the cell proliferation test different tumour cell lines (e.g. Du 145) can be examined. The cells were dispensed in RPMI 1640 culture medium, supplied with 10% (v/v) fetal calf serum plus 1% (v/v) Peniciltin/Streptomycin solution at a cell density of 2.000 cell/100 μL medium/per well (96well plate). After three hours the cells were washed with PBS (containing calcium and magnesium). 100 μl of culture medium above with 0.1% (v/v) fetal calf serum was added and cultured at 37° C. and 5% CO₂-atmosphere. Next day compounds of the present invention diluted in DMSO for appropriate concentrations were added and further 100 μL culture medium 0.5% (v/v) fetal calf serum. After 5 days cell culturing at 37° C. and 5% CO₂-atmosphere cells were washed with PBS. 20 μL of glutaraldehyde solution (11% (v/v)) is added and the cells were slightly shaken at room temperature for 15 min. After that the cell were washed 3 times and dried in the air. 100 μL of crystal violet solution (0.1% at pH 3.5) were added and the cells were shaken for 30 min. The cells were washed with tap water and air-dried. The colour is dissolved with 100 μL of acetic acid (10% (v/v)) under strong shaking for 5 min. The absorption was measured at 595 nm wavelength.

The compounds presented in this application have high potency activity as inhibitors of Tie2 kinase and/or Tie2 autophosphorylation as measured with the ELISA-method. The IC50 values are below 1 μM. At the same time the toxicity of the compounds is substantially lower which is different to other compounds in this structure class. Typically, the IC50 values determined in the DU 145 cytotoxicity assay are substantially higher as those determined in the Tie2 kinase or Tie2 autophosphorylation assay.

Certain compounds of the invention have been found be highly potent inhibitors of Tie2. More specifically, example compounds 1.4 to 1.7 throughout inhibit Tie2 with an IC50 of 1 μM or less either in the Tie2 kinase assay or in the Tie2 autophosphorylation ELISA test. While featuring high inhibitory potency against Tie2 kinase activity, certain compounds of the invention have been found to be particularly weakly cytotoxic or non-cytotoxic. More specifically, selected example compounds 1.4 to 1.7 showed IC50 values in the cytotoxicity assay using the cell line DU 145 which are at least five times higher as compared to those determined in the Tie2 kinase or Tie2 autophosphorylation assay.

Biological Experiment 4: Tie-2 Kinase Assay Without Preactivation of Kinase

A recombinant fusion protein of GST and the intracellular domains of Tie-2, expressed in insect cells (Hi-5) and purified by Glutathion-Sepharose affinity chromatography was used as kinase. Alternatively, commercially available GST-Tie2-fusion protein (Upstate Biotechnology, Dundee, Scotland) can be used. As substrate for the kinase reaction the biotinylated peptide biotin-Ahx-EPKDDAYPLYSDFG (C-terminus in amid form) was used which can be purchased e.g. from the company Biosynthan GmbH (Berlin-Buch, Germany). Tie-2 (3.5 ng/measurement point) was incubated for 60 min at 22° C. in the presence of 10 μM adenosine-tri-phosphate (ATP) and 1 μM substrate peptide (biotin-Ahx-EPKDDAYPLYSDFG-NH₂) with different concentrations of test compounds (0 μM and concentrations in the range 0.001-20 μM) in 5 μl assay buffer [50 mM Hepes/NaOH pH 7, 10 mM MgCl₂, 0.5 mM MnCl₂, 1.0 mM dithiothreitol, 0.01% NP40, protease inhibitor mixture (“Complete w/o EDTA” from Roche, 1 tablet per 2.5 ml), 1% (v/v) dimethylsulfoxide]. The reaction was stopped by the addition of 5 μl of an aqueous buffer ( 25 mM Hepes/NaOH pH 7.5, 0.28% (w/v) bovine serum albumin) containing EDTA (90 mM) and the HTRF (Homogeneous Time Resolved Fluorescence) detection reagents streptavidine-XLent (0.2 μM, from Cis Biointernational, Marcoule, France) and PT66-Eu-Chelate (0.3 ng/μl; a europium-chelate labelled anti-phospho-tyrosine antibody from Perkin Elmer).

The resulting mixture was incubated 1 h at 22° C. to allow the binding of the biotinylated phosphorylated peptide to the streptavidine-XLent and the PT66-Eu-Chelate. Subsequently the amount of phosphorylated substrate peptide was evaluated by measurement of the resonance energy transfer from the PT66-Eu-Chelate to the streptavidine-XLent. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate peptide. The data were normalised (enzyme reaction without inhibitor=0% inhibition, all other assay components but no enzyme=100% inhibition) and IC₅₀ values were calculated by a 4 parameter fit using an inhouse software.

Biological Experiment 5: Tie-2 Kinase Assay with Preactivation of Kinase

A recombinant fusion protein of GST and the intracellular domains of Tie-2, expressed in insect cells (Hi-5) and purified by Glutathion-Sepharose affinity chromatography was used as kinase. As substrate for the kinase reaction the biotinylated peptide biotin-Ahx-EPKDDAYPLYSDFG (C-terminus in amid form) was used which can be purchased e.g. from the company Biosynthan GmbH (Berlin-Buch, Germany).

For activation, Tie-2 was incubated at a conc. 12.5 ng/μl of for 20 min at 22° C. in the presence of 250 μM adenosine-tri-phosphate (ATP) in assay buffer [50 mM Hepes/NaOH pH 7, 10 mM MgCl₂, 0.5 mM MnCl₂, 1.0 mM dithiothreitol, 0.01% NP40, protease inhibitor mixture (“Complete w/o EDTA” from Roche, 1 tablet per 2.5 ml)].

For the subsequent kinase reaction, the preactivated Tie-2 (0.5 ng/measurement point) was incubated for 20 min at 22° C. in the presence of 10 μM adenosine-tri-phosphate (ATP) and 1 μM substrate peptide (biotin-Ahx-EPKDDAYPLYSDFG-NH₂) with different concentrations of test compounds (0 μM and concentrations in the range 0.001-20 μM) in 5 μl assay buffer [50 mM Hepes/NaOH pH 7, 10 mM MgCl₂, 0.5 mM MnCl₂, 0.1 mM sodium ortho-vanadate, 1.0 mM dithiothreitol, 0.01% NP40, protease inhibitor mixture (“Complete w/o EDTA” from Roche, 1 tablet per 2.5 ml), 1% (v/v) dimethylsulfoxide]. The reaction was stopped by the addition of 5 μl of an aqueous buffer (25 mM Hepes/NaOH pH 7.5, 0.28% (w/v) bovine serum albumin) containing EDTA (90 mM) and the HTRF (Homogeneous Time Resolved Fluorescence) detection reagents streptavidine-XLent (0.2 μM, from Cis Biointernational, Marcoule, France) and PT66-Eu-Chelate (0.3 ng/μl; a europium-chelate labelled anti-phospho-tyrosine antibody from Perkin Elmer). The resulting mixture was incubated 1 h at 22° C. to allow the binding of the biotinylated phosphorylated peptide to the streptavidine-XLent and the PT66-Eu-Chelate. Subsequently the amount of phosphorylated substrate peptide was evaluated by measurement of the resonance energy transfer from the PT66-Eu-Chelate to the streptavidine-XLent. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate peptide. The data were normalised (enzyme reaction without inhibitor =0% inhibition, all other assay components but no enzyme=100% inhibition) and IC₅₀ values were calculated by a 4 parameter fit using an inhouse software.

The compounds of the present invention are therefore preferentially active as antiangiogenesis inhibitors and not as cytostatic or cytotoxic agents that affect tumour cells and other proliferating tissue cells directly.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 05090098.4, filed Apr. 8, 2005, and U.S. Provisional Application Ser. No. 60/670,640, filed Apr. 13, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the general Formula I: 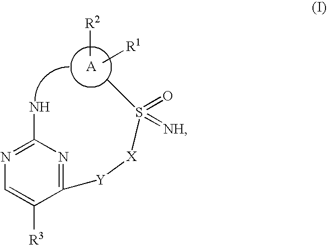
in which:
A is -phenylene- or —C₅-C₆-heteroarylene-;
X is a 2- to 6-membered alkylene tether which is unsubstituted or singly or multiply substituted with one or more substituents selected from the group comprising, preferably consisting of, halogen, C₁-C₄-alkoxy, hydroxy, amino, cyano, carboxy, —NH—C₁-C₄-alkyl, —N(C₁-C₄-alkyl)₂, and C₁-C₄-alkyl, which itself may be unsubstituted or singly or multiply substituted by halogen, C₁-C₄-alkoxy, hydroxy, amino, cyano, carboxy, —NH-C₁-C₄-alkyl, or —N(C₁-C₄-alkyl)2;
Y is —NR⁴—, —O—, or —S—;
R¹ and R² are the same or different and are independently from each other selected from the group comprising, preferably consisting of, hydrogen, hydroxy, halogen, nitro, cyano, —(CH₂)ₚP(O)(OR⁵)₂, —(CH₂)ₚOP(O)(OR⁵)₂—(CH₂)ₚ—NR⁵R⁶, —(CH₂)ₚ—NR⁵C(O)R⁶, —(CH₂)ₚ—NR⁵C(S)R⁶, —(CH₂)ₚ—NR⁵S(O)R⁶, —(CH₂)ₚ—NR⁵S(O)₂R⁶, —(CH₂)ₚ—NR⁵C(O)NR⁶R⁷, —(CH₂)ₚ—NR⁵C(O)OR⁶, —(CH₂)ₚ—NR⁵C(NH)NR⁶R⁷, —(CH₂)ₚ—NR⁵C(S)NR^{b 6}R⁷, —(CH₂)ₚ—NR⁵S(O)NR⁶R⁷, —(CH₂)ₚ—NR⁵S(O)₂NR⁶R⁷, —(CH₂)ₚ—C(O)R⁵, —(CH₂)ₚ—CR⁴(OH)—R⁵, —(CH₂)ₚ—C(S)R⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—S(O)₂N═CH—NR⁵R⁶, —(CH₂)ₚ—SO₃R⁵, —(CH₂)ₚ—CO₂R⁵, —(CH₂)ₚ—C(O)NR⁵R⁶, —(CH₂)ₚ—C(S)NR⁵R⁶, —(CH₂)ₚ—SR⁵, —(CH₂)ₚ—OR⁵, —CR⁵(OH)—R⁶, —O—(CH₂)ₚ—C(O)R⁵, —O—(CH₂)ₚ—CR⁴(OH)—R⁵, and moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₆-heteroaryl, -phenylene-(CH₂)ₚ—R⁵, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl, and wherein 0 to 2 methylene groups of —C₁-C₈-alkyl may be replaced independently of each other by —O—, —S—, —C(═O), —SO₂— or —NR⁴—;
R³ is selected from the group comprising, preferably consisting of, halogen, nitro, cyano, —C₁-C₆-alkylthio, amino, —NH—(CH₂)ₚ—C₃-C₈-cycloalkyl, benzodioxolyl, —(CH₂)ₚP(O)(OR⁵)₂, —NR⁵R⁶, —S(O)(C₁-C₆-alkyl), —S(O)₂(C₁-C₆-alkyl), —C(O)NR⁵R⁶, —C(O)R⁵, —C(O)OR⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₁₀-heteroaryl, —O-phenyl, —O—C₅-C₁₀-heteroaryl, -phenylene—(CH₂)ₚ—R⁵, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —NH—(CH₂)ₚ—C₃-C₈-cycloalkyl, —C₃-C₈-cycloalkyl, —C₃-C₈-heterocycloalkyl, —(CH₂)ₚ-phenyl, —(CH₂)ₚ—C₅-C₆-heteroaryl, -phenylene-(CH₂)ₚ—R⁵, —NR⁵R⁶, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —C₁-C₆-alkoxy-C₁-C₆-alkoxy-C₁-C₆-alkyl, —(CH₂)ₚP(O)(OR⁵)₂, —(CH₂)ₚOP(O)(OR⁵)₂, —(CH₂)ₚ—NR⁵C(O)NR⁶R⁷, —(CH₂)ₚ—S(O)R⁵, —(CH₂)ₚ—S(O)₂R⁵, —(CH₂)ₚ—NR⁵S(O)₂R⁶, —(CH₂)ₚ—S(O)₂NR⁵R⁶, —(CH₂)ₚ—C(O)NR⁵R⁶, —(CH₂)ₚ—NR⁵C(O)R⁶, —(CH₂)ₚ—CR⁴(OH)—R⁵, —O—(CH₂)ₚ—CR⁴(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein phenylene, —C₃-C₈-cycloalkyl, —C₃-C₈-heterocycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —SO₂— or —NR⁴—;
R⁴ is hydrogen or —C₁-C₈-alkyl;
R⁵, R⁶,
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —NH—(CH₂)ₚ—C₃-C₈-cycloalkyl, —C₃-C₈-cycloalkyl, —C₃-C₈-heterocycloalkyl, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —C₁-C₆-alkoxy-C₁-C₆-alkoxy-C₁-C₆-alkyl, —NR⁹R¹⁰, NR⁹C(O)NR¹⁰, —S(O)(C₁-C₆-alkyl), —S(O)₂(C₁-C₆-alkyl), —S(O)₂-phenyl, —NH—S(O)₂(C₁-C₆-alkyl), —NH—S(O)₂-phenyl, —S(O)₂—NH—(C₁-C₆-alkyl), —S(O)₂—NH-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₁₀-heteroaryl, -phenylene-(CH₂)ₚ—R⁹, (CH₂)ₚP(O)(OR⁹)₂, —(CH₂)ₚOP(O)(OR⁹)₂, wherein phenylene, —C₃-C₈-cycloalkyl, —C₃-C₈-heterocycloalkyl, phenyl, —(CH₂)ₚ-phenyl and —(CH₂)ₚ—C₅-C₁₀-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 2- to 8-membered alkylene tether which may be unsubstituted or singly substituted by —(CH₂)ₚ—OH, —(CH₂)ₚ—CN, or —(CH₂)ₚ—NR⁹R¹⁰, and in which 0 to 2 methylene groups may be replaced by —O—, —S—, —(C═O)—, —S(O)₂—, or —NR⁴—;
R⁹, R¹⁰ are independently from each other hydrogen or —C₁-C₈-alkyl;
p is an integer of 0, 1, 2, 3, 4, 5, or 6; and solvates, hydrates, N-oxides, isomers and salts thereof.

2. The compound according to claim 1, wherein:
A is -meta-phenylene- or —C₅-C₆-heteroarylene-;
X is a 2- to 6-membered alkylene tether which is unsubstituted or singly or multiply substituted with one or more substituents selected from the group comprising, preferably consisting of, C₁-C₄-alkoxy, hydroxy, amino, —NH-C₁-C₄-alkyl, —N(C₁-C₄-alkyl)₂, or C₁-C₄-alkyl, which itself may be unsubstituted or singly or multiply substituted by C₁-C₄-alkoxy, hydroxy, amino, —NH—C₁-C₄-alkyl, or —N(C₁-C₄-alkyl)₂;
Y is —NR⁴— or —O—;
R¹ and R² are the same or different and are independently from each other selected from the group comprising, preferably consisting of, hydrogen, hydroxy, halogen, nitro, cyano, —(CH₂)ₚ—NR⁵R⁶, —(CH₂)ₚ—NR⁵C(O)R⁶, —(CH₂)ₚ—NR⁵S(O)₂R⁶, —(CH₂)ₚ—NR⁵C(O)NR⁶R⁷, —(CH₂)ₚ—NR⁵C(O)OR⁶, —(CH₂)ₚ—C(O)R⁵, —(CH₂)ₚ—CR⁴(OH)—R⁵, (CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —(CH₂)ₚ—OR⁵, —CR⁵(OH)—R⁶, —O—(CH₂)ₚ—C(O)R⁵, —O—(CH₂)ₚ—CR⁴(OH)—R⁵, and moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl, and wherein 0 to 2 methylene groups of —C₁-C₈-alkyl may be replaced independently of each other by —O—, —S—, —C(═O)—, —S(O)₂— or —NR⁴—;
R³ is selected from the group comprising, preferably consisting of, halogen, nitro, cyano, —C₁-C₆-alkylthio, amino, benzodioxolyl, —NR⁵R⁶, —S(O)₂(C₁-C₆-alkyl), —C(O)NR⁵R⁶, —C(O)R⁵, —C(O)OR⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₁₀-heteroaryl, —O-phenyl, —O—C₅-C₁₀-heteroaryl, -phenylene—(CH₂)ₚ—R⁵, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —C₃—C₈-cycloalkyl, —C₃-C₈-heterocycloalkyl, —(CH₂)ₚ-phenyl, —(CH₂)ₚ—C₅-C₆-heteroaryl, -phenylene-(CH₂)ₚ—R⁵, —NR⁵R⁶, —C₁-C₆-hydroxyalkyl, —C₁-C₆-haloalkyl, —C₁-C₆-haloalkoxy, —C₂-C₆-alkenyl, —C₂-C₆-alkynyl, —C₁-C₆-alkoxy-C₁-C₆-alkyl, —(CH₂)ₚ—NR⁵C(O)NR⁶R⁷, —(CH₂)ₚ—S(O)₂R⁵, —(CH₂)ₚ—NR⁵S(O)₂R⁶, —(CH₂)ₚ—S(O)₂NR⁵R⁶, —(CH₂)ₚ—C(O)NR⁵R⁶, —(CH₂)ₚ—NR⁵C(O)R⁶, —(CH₂)ₚ—CR⁴(OH)—R⁵, —O—(CH₂)ₚ—CR⁴(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein phenylene, —C₃-C₈-cycloalkyl, —C₃-C₈-heterocycloalkyl, —(CH₂)ₚ-phenyl and —(CH₂)ₚ—C₅-C₁₀-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—;
R⁴ is hydrogen or —C₁-C₈-alkyl;
R⁵, R⁶,
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, —C₁-C₆-alkylthio, amino, cyano, —C₁-C₈-alkyl, —C₃-C₈-cycloalkyl, —C₃-C₈-heterocycloalkyl, —C₁-C₆-hydroxyalkyl, —C₁-C₆-haloalkyl, —C₁-C₆-haloalkoxy, —NR⁹R¹⁰, —NR⁹C(O)NR¹⁰, —S(O)₂(C₁-C₆-alkyl), —S(O)₂-phenyl, —NH—S(O)₂(C₁-C₆-alkyl), —NH—S(O)₂-phenyl, —S(O)₂—NH—(C₁-C₆-alkyl), —S(O)₂—NH-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₆-heteroaryl, -phenylene—(CH₂)ₚ—R⁹, wherein phenylene, —C₃-C₈-cycloalkyl, —C₃-C₈-heterocycloalkyl, phenyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 2- to 8-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—;
R⁹, R¹⁰ are independently from each other hydrogen or —C₁-C₈-alkyl;
p is an integer of 0, 1, 2, 3, 4, 5, or 6.

3. The compound according to claim 1, wherein:
A is -meta-phenylene-;
X is an unsubstituted 4- to 5-membered alkylene tether;
Y is —NR⁴— or —O—;
R¹ and R² are the same or different and are independently from each other selected from the group comprising, preferably consisting of, hydrogen, hydroxy, halogen, nitro, cyano, —(CH₂)ₚ—NR⁵R⁶, —(CH₂)ₚ—NHC(O)R⁶, —(CH₂)ₚ—NHS(O)₂R⁶, —(CH₂)ₚ—NHC(O)NR⁶R⁷, (CH₂)ₚ—NHC(O)OR⁶, —(CH₂)ₚ—C(O)R⁵, —(CH₂)ₚ—CH(OH)—R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —(CH₂)ₚ—OR⁵, —CR⁵(OH)—R⁶, —O—(CH₂)ₚ—C(O)R⁵, —O—(CH₂)ₚ—CH(OH)—R⁵ and moieties being selected from the group comprising, preferably consisting of, —C₁-C₈-alkyl, —(CH₂)ₚ-phenyl, —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl;
R³ is selected from the group comprising, preferably consisting of, halogen, cyano, amino, benzodioxolyl, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷, and moieties, said moieties being selected from the group comprising, preferably consisting of, —C₁-C₈-alkyl, —C₂-C₈-alkenyl, —C₂-C₈-alkynyl, —C₃-C₈-cycloalkyl, —C₃-C₈-heterocycloalkyl, —(CH₂)ₚ-phenyl, —(CH₂)ₚ—C₅-C₁₀-heteroaryl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ-phenyl and —(CH₂)ₚC₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—;
R⁴ is hydrogen or —C₁-C₈-alkyl;
R⁵, R⁶,
R⁷ and R⁸ are the same or different and are independently from each other selected from the group comprising, preferably consisting of, hydrogen, benzodioxolyl and moieties, said moieties being selected from the group comprising, preferably consisting of, —C₁-C₈-alkyl, —C₃-C₈-cycloalkyl, —C₃-C₈-heterocycloalkyl, phenyl, —C₅-C₁₀-heteroaryl, wherein said moieties are unsubstituted or singly or multiply substituted independently from each other with hydroxy, halogen, —C₁-C₆-alkoxy, amino, cyano, —C₁-C₈-alkyl, —C₃-C₈-cycloalkyl, —C₁-C₆-hydroxyalkyl, —C₁-C₆-haloalkyl, —C₁-C₆-haloalkoxy, —NR⁹R¹⁰, —NR⁹C(O)NR¹⁰, —S(O)₂(C₁-C₆-alkyl), —S(O)₂-phenyl, —NH—S(O)₂(C₁-C₆-alkyl), —NH—S(O)₂-phenyl, —C₁-C₆-alkanoyl, —C(O)NR⁹R¹⁰, —C(O)R⁹, —C(O)OR⁹, —(CH₂)ₚ-phenyl, —(CH₂)ₚ—C₅-C₆-heteroaryl, wherein —C₃-C₈-cycloalkyl, phenyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃, and wherein 0 to 2 methylene groups of —C₁-C₈-alkyl may be replaced independently of each other by —O—, —S—, —C(═O)—, —S(O)₂— or —NR⁴—; or two members selected from the group comprising, preferably consisting of, R⁵, R⁶, R⁷ and R⁸ form a 3- to 7-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—;
R⁹, R¹⁰ are independently from each other hydrogen or —C₁-C₈-alkyl;
p is an integer of 0, 1, 2, 3, or 4.

4. The compound according to claim 1, wherein:
R⁴ is hydrogen, methyl, or ethyl.

5. The compound according to claim 1, wherein:
R⁵, R⁶,
R⁷ and R⁸ are the same or different and are independently from each other selected from the group comprising, preferably consisting of, hydrogen and moieties, said moieties being selected from the group comprising, preferably consisting of, —C₃-C₈-cycloalkyl, —C₃-C₈-heterocycloalkyl, —C₁-C₈-alkyl, phenyl and —C₅-C₆-heteroaryl, wherein said moieties are unsubstituted or singly or multiply substituted independently from each other with hydroxy, halogen, —C₁-C₆-alkoxy, amino, cyano, —C₁-C₆-alkyl, —C₁-C₆-hydroxyalkyl, —C₁-C₆-haloalkyl, —C₁-C₆-haloalkoxy, —NR⁹R¹⁰, —C(O)NR⁹R¹⁰, —C(O)R⁹, or two members selected from the group comprising, preferably consisting of, R⁵, R⁶, R⁷ and R⁸ form a 3- to 7-membered alkylene tether which may be unsubstituted or singly substituted by —(CH₂)ₚ—OH or —(CH₂)ₚ—NR⁹R¹⁰, and in which 0 to 2 methylene groups may be replaced by —O— or —NR⁴—.

6. The compound according to claim 1, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷, —(CH₂)ₚ—NHC(O)OR⁶, —(CH₂)ₚ—C(O)R⁵, —(CH₂)ₚ—CH(OH)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—CO₂R⁵, —(CH₂)ₚ—C(O)NR⁵R⁶, —CR⁵(OH)—R⁶, —O—(CH₂)ₚ—C(O)R⁵, —O—(CH₂)ₚ—CH(OH)—R⁵.

7. The compound according to claim 1, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷, —(CH₂)ₚ—NHC(O)OR⁶, —(CH₂)ₚ—C(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—C(O)NR⁵R⁶, —O—(CH₂)ₚ—C(O)R⁵.

8. The compound according to claim 1, wherein:
R¹ and R² are the same or different and are independently from each other selected from the group comprising, preferably consisting of, hydrogen, —(CH₂)ₚ—NHC(O)R⁶, —(CH₂)ₚ—NHS(O)₂R⁶, —(CH₂)ₚ—NHC(O)NR⁶R⁷.

9. The compound according to claim 1, wherein:
R¹ and R² are the same or different and are independently from each other selected from the group comprising, preferably consisting of, —C₁-C₈-alkyl, —(CH₂)ₚ-phenyl. —(CH₂)ₚ—C₅-C₆-heteroaryl, wherein said moieties are unsubstituted or singly or multiply substituted independently from each other with hydroxy, halogen, phenyl, —NR⁵R⁶, cyano, —C₁-C₄-alkyl, —C₁-C₄-alkoxy, —C₁-C₄-haloalkyl, —C₁-C₄-haloalkoxy, or —C₁-C₄-hydroxyalkyl.

10. The compound according to claim 1, wherein:
R³ is selected from the group comprising, preferably consisting of, halogen, cyano, amino, benzodioxolyl, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷.

11. The compound according to claim 1, wherein:
R³ is selected from the group comprising, preferably consisting of, —NR⁵R⁶, —C(O)NR⁵R⁶, —C(O)R⁵, —NR⁵—(CH₂)ₚ—NR⁶C(O)NR⁷R⁸, —NR⁵—(CH₂)ₚ—NR⁶S(O)₂R⁷, —NR⁵—(CH₂)ₚ—NR⁶C(O)R⁷, —NR⁵—(CH₂)ₚ—C(O)NR⁶R⁷ .

12. The compound according to claim 1, wherein:
R³ is selected from the group comprising, preferably consisting of, halogen, cyano, amino.

13. The compound according to claim 1, wherein:
R³ is benzodioxolyl.

14. The compound according to claim 1, wherein:
R³ is selected from the group comprising, preferably consisting of, —C₁-C₈-alkyl, —C₂-C₈-alkenyl, and —C₂-C₈-alkynyl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

15. The compound according to claim 1, wherein:
R³ is selected from the group comprising, preferably consisting of, —C₁-C₈-alkyl and —C₂-C₈-alkynyl, wherein said moieties are unsubstituted or singly or multiply substituted independently from each other with halogen, —C₁-C₆-alkoxy, C₁-C₆-alkyl, —(CH₂)ₚ-phenyl, —(CH₂)ₚ—C₅-C₆-heteroaryl, —NR⁵R⁶, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —(CH₂)ₚ—C(O)R⁵, wherein —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

16. The compound according to claim 1, wherein:
R³ is selected from the group comprising, preferably consisting of, —C₃-C₈-cycloalkyl and —C₃-C₈-heterocycloalkyl, and is unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

17. The compound according to claim 1, wherein:
R³ is selected from the group comprising, preferably consisting of, cyclopentyl, cyclohexyl, piperazinyl, piperidinyl, 1,4-diazepanyl and pyrrolidinyl, wherein said moieties are unsubstituted or singly or multiply substituted independently from each other with hydroxy, —C₁-C₆-alkoxy, cyano, —C₁-C₆-alkyl, —C₃-C₈-cycloalkyl, —C₃-C₈-heterocycloalkyl, —NR⁵R⁶, —C₁-C₆-hydroxyalkyl, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

18. The compound according to claim 1, wherein:
R³ is selected from the group comprising, preferably consisting of, piperazinyl, piperidinyl, and pyrrolidinyl, wherein said moieties are unsubstituted or singly or multiply substituted independently from each other with —C₁-C₆-alkyl, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

19. The compound according to claim 1, wherein:
R³ is piperazinyl and is unsubstituted or singly or multiply substituted independently from each other with —C₁-C₆-alkyl, —(CH₂)ₚ—S(O)₂R⁵, —(CH₂)ₚ—C(O)NR⁵R⁶, —(CH₂)ₚ—CH(OH)—R⁵, —(CH₂)ₚ—C(O)R⁵.

20. The compound according to claim 1 wherein:
R³ is selected from the group comprising, preferably consisting of, —(CH₂)ₚ-phenyl and —(CH₂)ₚ—C₅-C₁₀-heteroaryl, wherein said moieties are unsubstituted or singly or multiply substituted independently from each other with hydroxy, halogen, —C₁-C₆-alkoxy, amino, cyano, —C₁-C₆-alkyl, —C₃-C₈-cycloalkyl, —C₃-C₈-heterocycloalkyl, —(CH₂)ₚ-phenyl, —(CH₂)ₚ—C₅-C₆-heteroaryl, —NR⁵R⁶, —C₁-C₆-hydroxyalkyl, —C₁-C₆-haloalkyl, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —(CH₂)ₚ—S(O)₂NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —(CH₂)ₚ—C(O)OR⁵, —O—(CH₂)ₚ—R⁵, wherein —C₃-C₈-cycloalkyl, —C₃-C₈-heterocycloalkyl, —(CH₂)ₚ-phenyl and —(CH₂)ₚ—C₅-C₆-heteroaryl are unsubstituted or singly or multiply substituted independently from each other with halogen, hydroxy, —C₁-C₆-alkyl, —C₁-C₆-alkoxy, —CF₃ and/or —OCF₃.

21. The compound according to claim 1, wherein:
R³ is selected from the group comprising, preferably consisting of, —(CH₂)ₚ-phenyl and —(CH₂)ₚ—C₅-C₆-heteroaryl, wherein said moieties are unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—S(O)₂R⁵, —(CH₂)ₚ—NHS(O)R⁶, —(CH₂)ₚ—S(O)NR⁵R⁶, —(CH₂)ₚ—C(O)NR⁵R⁶, —(CH₂)ₚ—NHC(O)R⁶, —(CH₂)ₚ—CH(OH)—R⁵, —O—(CH₂)ₚ—CH(OH)—R⁵, —(CH₂)ₚ—C(O)R⁵, —O—(CH₂)ₚ—C(O)R⁵, —O—(CH₂)ₚ—R⁵.

22. The compound according to claim 1, wherein:
R³ is selected from the group comprising, preferably consisting of, phenyl and —C₅-C₆-heteroaryl, wherein said moieties are unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—S(O)₂R⁵, —(CH₂)ₚ—NHS(O)₂R⁶, —CH₂)ₚ—S(O)NR⁵R⁶, —(CH₂)ₚ—C(O)NR⁵R⁶, —(CH₂)ₚ—NHC(O)R⁶, —(CH₂)ₚ—CH(OH)R⁵, —O—(CH₂)ₚ—CH(OH)R⁵, —(CH₂)ₚ—C(O)R⁵, —O—(CH₂)ₚ—C(O)R⁵, —O—(CH₂)ₚ—R⁵.

23. The compound according to claim 1, wherein:
R³ is phenyl and is unsubstituted or singly or multiply substituted independently from each other with halogen, —C₁-C₆-alkoxy, cyano, —C₁-C₆-alkyl, —NR⁵R⁶, —C₁-C₆-haloalkoxy, —(CH₂)ₚ—NHC(O)NR⁶R⁷, —(CH₂)ₚ—NHS(O)₂R⁶, —(CH₂)ₚ—C(O)NR⁵R⁶, —(CH₂)ₚ—NHC(O)R⁶, —(CH₂)ₚ—C(O)R⁵, —O—(CH₂)ₚ—C(O)R⁵.

24. The compound according to claim 1 any one of claims 1, wherein:
R¹, R² is hydrogen, —NO₂, —NH₂, —NHC(O)NR⁵R⁶, —NHC(O)R⁵;
X is an unsubstituted 4-membered tether;
Y is —NH—; and
R³ is a phenyl group which is optionally substituted one or more times with, independently of each other, Hal, C₁-C₆-alkyl, C₁-C₆-alkoxy, —NHC(O)NR⁵R⁶, —NHS(O)₂R⁵, or —NHC(O)R⁵.

25. The compound according to claim 1, which is selected from the group consisting of:
(RS)-1⁵-Bromo-4-imino-4-thia-2,9-diaza-1(2,4)-pyrimidina-3(1,3)-benzenacyclononaphane-4-oxide;
(RS)-1⁵-Iodo-4-imino-4-thia-2,9-diaza-1(2,4)-pyrimidina-3(1,3)-benzenacyclononaphane-4
(RS)-4-Imino-1⁵-(4-methoxyphenyl)-4-thia-2,9-diaza-1(2,4)-pyrimidina-3(1,3)-benzenacyclononaphane 4-oxide;
N-{4-[(RS)-4-Imino-4-oxo-4λ⁶-thia-2,9-diaza-1(2,4)-pyrimidina-3(1,3)-benzenacyclononaphan-1⁵-yl]phenyl}-N′-phenyl urea;
N-{4-[(RS)-4-Imino-4-oxo-4λ⁶-thia-2,9-diaza-1(2,4)-pyrimidina-3(1,3)-benzenacyclononaphan-1⁵-yl]phenyl}-N′-[3-(trifluormethyl)phenyl]urea;
N-{4-[(RS)-4-Imino-4-oxo-4λ⁶-thia-2,9-diaza-1(2,4)-pyrimidina-3(1,3)-benzenacyclononaphan-1⁵-yl]phenyl}-N′-(3-methylphenyl)urea;
N-(3-Ethylphenyl)-N′-{4-[(RS)-4-imino-4-oxo-4λ⁶-thia-2,9-diaza-1(2,4)-pyrimidina-3(1,3)-benzenacyclononaphan-1⁵-yl]phenyl}urea;
(RS)-1-[2-Fluoro-5-(trifluoromethyl)phenyl]-3-{4-[4-imino-4-oxo-4λ⁶-thia-2,9-diaza-1(2,4)-pyrimidina-3(1,3)-benzenacyclononaphan-1⁵-yl]phenyl}urea;
(RS)-4-Imino-1⁵-(2-methyl-4-methoxyphenyl)-4-thia-2,9-diaza-1(2,4)pyrimidina-3(1,3)-benzenacyclononaphane 4-oxide;
(RS)-2,3-Dichloro-N-{4-[4-imino-4-oxo-4-thia-2,9-diaza-1(2,4)pyrimidina-3( 1,3)-benzenacyclononaphan-1⁵-yl]phenyl}benzenesulfonamide;
(RS)-4-Imino-1⁵-[4-(1-methylethoxy)phenyl]-4-thia-2,9-diaza-1(2,4)-pyrimidina-3(1,3)-benzenacyclononaphane 4-oxide;
(RS)-1⁵-(4-Ethylphenyl)-4-imino-4-thia-2,9-diaza-1(2,4)-pyrimidina-3(1,3)-benzenacyclononaphane 4-oxide;
(RS)-1⁵-(4-Ethoxyphenyl)-4-imino-4-thia-2,9-diaza-1(2,4)-pyrimidina-3(1,3)-benzenacyclononaphane 4-oxide;
(RS)-1⁵-(3-Fluoro-4-methoxyphenyl)-4-imino-4-thia-2,9-diaza-1(2,4)-pyrimidina-3(1,3)-benzenacyclononaphane 4-oxide;
(RS)-1⁵-(4-Ethoxy-3-fluorophenyl)-4-imino-4-thia-2,9-diaza-1(2,4)-pyrimidina-3(1,3)-benzenacyclononaphane 4-oxide;
(RS)—N-{4-[4-Imino-4-oxo-4-thia-2,9-diaza-1(2,4)-pyrimidina-3(1,3)-benzenacyclononaphan 1⁵-yl]phenyl}-1-phenylcyclopropanecarboxamide;
(RS)-2,3-Dichloro-N-{4-[4-imino-4-oxo-4-thia-2,9-diaza-1(2,4)-pyrimidina-3(1,3)-benzenacyclononaphan-1⁵-yl]but-3-ynyl}benzenesulfonamide;
2-{4-[(RS)-4-Imino-4-oxo-4λ⁶-thia-2,9-diaza-1(2,4)-pyrimidina-3(1,3)-benzenacyclononaphan-1⁵-yl]phenoxy}-1-(2,4-xylyl)ethan-1-one;
(RS)-4-Imino-1⁵-iodo-3⁵-nitro-4-thia-2,9-diaza-1(2,4)-pyrimidina-3(1,3)-benzenacyclononaphane 4-oxide;
N—[(RS)-4-Imino-1⁵-iodo-4-oxo-4λ⁶-thia-2,9-diaza-1(2,4)-pyrimidina-3(1,3)-benzenacyclononaphan-3⁵-yl]pyrrolidine-1-carboxamide; and
N—[(RS)-1⁵-(4-Ethoxyphenyl)-4-imino-4-oxo-4λ⁶-thia-2,9-diaza-1(2,4)-pyrimidina-3(1,3)-benzenacyclononaphan-3⁵-yl]pyrrolidine-1-carboxamide.

26. A method of preparing the compound of Formula I according to claim 1, wherein an intermediate compound of general formula C: 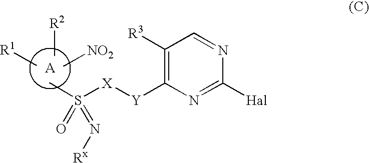
in which R^{x} is selected from the group comprising, preferably consisting of, hydrogen, —C(O)OR⁵, —C(═O)R⁵, C(═O)NR⁵R⁶, —S(O)₂R⁵, and —S(O)₂(CH₂)ₚ—Si(C₁-C₄-alkyl)₃, in which p, R⁵ and R⁶ have the same meaning as in claim 1,
is converted, for example by reduction with a suitable reducing agent, then intramolecular substitution with a suitable substituent, e.g. Hal, and, if R^{x} is not hydrogen, cleavage of R^{x}, to a compound of general formula (I): 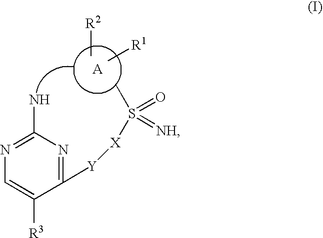
in which compounds A, X, Y, R¹, R², R³ and Hal have the meaning as defined in claim 1.

27. The method according to claim 26, wherein said intermediate compound of general formula (C) is prepared by allowing an intermediate compound of formula (B): 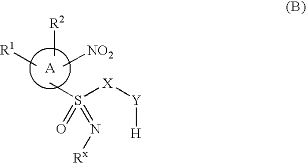
to react with a compound of formula (D): 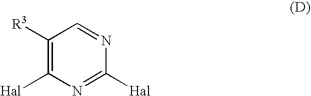
in which compounds A, X, Y, R¹, R², and R³, have the same meaning as in Formula I in claim 26, and in which R^{x} has the same meaning as in Formula C in claim 26.

28. The method according to claim 27, wherein said intermediate compound of general formula (B) is prepared by allowing a disulphide compound of formula (A): 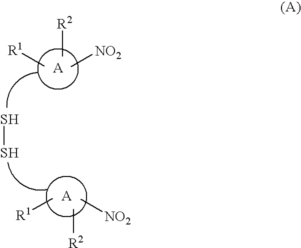
to undergo successive steps of reduction of the disulphide bridge, alkylation, oxidation, and then sulphoximine formation, thus producing an intermediate of general formula (A′): 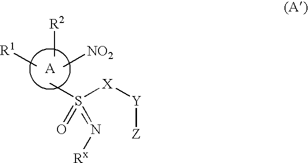
which intermediate (A′) is then allowed to undergo successive transformation or deprotection, thus producing said intermediate compound of general formula (B),
in which compounds A, X, Y, R¹, and R², have the same meaning as in Formula (I), and Z is a suitable protecting or activating group, and in which R^{x} has the same meaning as in Formula C.

29. An intermediate compound of general formula (Ia): 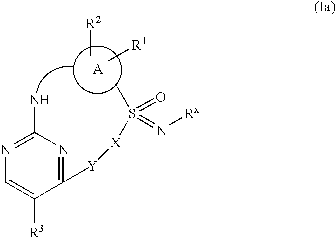
in which R^{x} is selected from the group comprising, preferably consisting of, —C(O)OR⁵, —C(═O)R⁵, C(═O)NR⁵R⁶, —S(O)R⁵, and —S(O)₂(CH₂)ₚ—Si(C₁-C₄-alkyl)₃, in which p, R⁵ and R⁶ have the same meaning as in claim 1, and in which A, X, Y, R¹, R², and R³ have the meaning as defined in claim 1.

30. An intermediate compound of general formula (C): 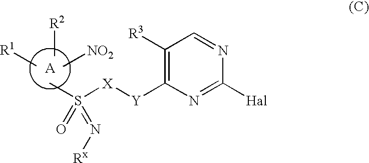
in which, A, X, Y, R¹, R², and R³ have the same meaning as in Formula (I) in claim 26, and Hal has the same meaning as, and in which R^{x} has the same meaning as in Formula C in claim 26.

31. An intermediate compound of general formula (B): 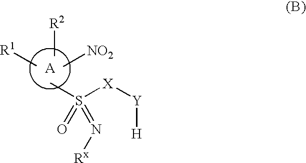
in which, A, X, Y, R¹, and R², have the same meaning as in Formula (I) in claim 26, and in which R^{x} has the same meaning as in Formula C in claim 26.

32. An intermediate compound of general formula (A′): 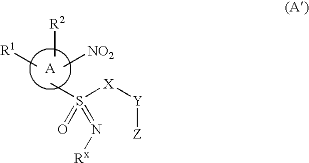
in which, A, X, Y, Z, R¹, and R² have the same meaning as in Formula (I) in claim 26, and in which R^{x} has the same meaning as in Formula C in claim 26.

33. An intermediate compound of general formula (A): 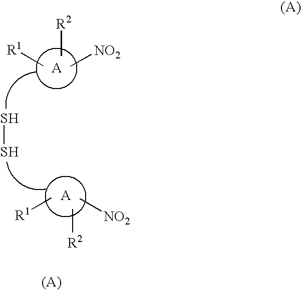
in which, A, R¹, and R², have the same meaning as in Formula I in claim 26.

34. Use of an intermediate compound of general formula (Ia) for the preparation of a compound of general formula (I) according to claim 1.

35. Use of intermediate compound of general formula (C) for the preparation of a compound of general formula (I) according to claim 1.

36. Use of intermediate compound of general formula (B) for the preparation of a compound of general formula (I) according to claim 1.

37. Use of intermediate compound of general formula (A′) of claim 32 for the preparation of a compound of general formula (1) according to any one of claims 1 to 25.

38. Use of intermediate compound of general formula (A) for the preparation of a compound of general formula (I) according to claim 1.

39. A pharmaceutical composition which comprises a compound of the Formula (I) according to claim 1 or an in vivo hydrolysable ester thereof, and one or more pharmaceutically-acceptable diluent or carrier.

40. Use of the compound of the Formula I according to claim 1 for manufacturing a pharmaceutical composition for the treatment of diseases of dysregulated vascular growth or of diseases which are accompanied with dysregulated vascular growth.

41. The use according to claim 40, wherein the diseases are retinopathy, other angiogenesis dependent diseases of the eye, rheumatoid arthritis, and other inflammatory diseases associated with angiogenesis.

42. The use according to claim 41, wherein the other angiogenesis dependent diseases of the eye are cornea transplant rejection, age-related macular degeneration.

43. The use according to claim 41, wherein the inflammatory diseases associated with angiogenesis are psoriasis, delayed type hypersensitivity, contact dermatitis, asthma, multiple sclerosis, restenosis, pulmonary hypertension, stroke, and diseases of the bowel.

44. The use according to claim 40, wherein the diseases are coronary and peripheral artery disease.

45. The use according to claim 40, wherein the diseases are ascites, oedema such as brain tumour associated oedema, high altitude trauma, hypoxia induced cerebral oedema pulmonary oedema and macular oedema or oedema following burns and trauma, chronic lung disease, adult respiratory distress syndrome, bone resorption and for benign proliferating diseases such as myoma, benign prostate hyperplasia and wound healing for the reduction of scar formation, reduction of scar formation during regeneration of damaged nerves, endometriosis, pre-eclampsia, postmenopausal bleeding and ovarian hyperstimulation.

46. The use according to claim 40, wherein the disease is a solid tumour and/or metastases thereof.

47. A method for treating a disease of dysregulated vascular growth or diseases which are accompanied with dysregulated vascular growth.

48. The method according to claim 47, wherein the diseases are retinopathy, other angiogenesis dependent diseases of the eye rheumatoid arthritis, and other inflammatory diseases associated with angiogenesis.

49. The method according to claim 48, wherein the angiogenesis dependent diseases of the eye are cornea transplant rejection, age-related macular degeneration.

50. The method according to claim 48, wherein the inflammatory diseases associated with angiogenesis are psoriasis, delayed type hypersensitivity, contact dermatitis, asthma, multiple sclerosis, restenosis, pulmonary hypertension, stroke, and diseases of the bowel.

51. The method according to claim 47, wherein the diseases are coronary and peripheral artery disease.

52. The method according to claim 47, wherein the diseases are ascites, oedema such as brain tumour associated oedema, high altitude trauma, hypoxia induced cerebral oedema pulmonary oedema and macular oedema or oedema following burns and trauma, chronic lung disease, adult respiratory distress syndrome, bone resorption and for benign proliferating diseases such as myoma, benign prostate hyperplasia and wound healing for the reduction of scar formation, reduction of scar formation during regeneration of damaged nerves, endometriosis, pre-eclampsia, postmenopausal bleeding and ovarian hyperstimulation.

53. The method according to claim 47, wherein the disease is a solid tumour and/or metastases thereof.
